SEQUENCE LISTING (1 sequence)

SEQ ID NO: 1 - protein, 13 aa, Artificial Sequence
  alpha-bungarotoxin (BTX) peptide
MRYYESSLKSYPD

(19) United States
(12) Patent Application Publication
(10) Pub. No.: US 2023/0096053 A1
(43) Pub. Date: Mar. 30, 2023

(54) GENETICALLY-TARGETED CHEMICAL ASSEMBLY: BUILDING FUNCTIONAL STRUCTURES AND MATERIALS IN LIVING CELLS, TISSUES, AND ANIMALS

(71) Applicant: The Board of Trustees of the Leland Stanford Junior University, Stanford, CA (US)

(72) Inventors: Karl A. Deisseroth, Stanford, CA (US); Zhenan Bao, Stanford, CA (US); Jia Liu, Stanford, CA (US); Charu Ramakrishnan, Stanford, CA (US); Yoon Seok Kim, Stanford, CA (US); Ariane C. Tom, Stanford, CA (US)

(21) Appl. No.: 17/782,465

(22) PCT Filed: Dec. 4, 2020
(86) PCT No.: PCT/US2020/063403

§ 371 Date: Jun. 3, 2022

Related U.S. Application Data
(60) Provisional application No. 62/944,578, filed on Dec. 6, 2019.

(51) Int. Cl. A61L 27/26; C12N 9/08; C12N 15/62; C12N 15/86; C12N 5/0793; G01N 33/50; C07K 14/405; A61N 1/32

(52) U.S. Cl. CPC A61L 27/26; C12N 9/0065; C12N 15/625; C12N 15/86; C12N 5/0619; G01N 33/5005; C07K 14/405; A61N 1/326; C12N 2750/14143; C07K 2319/60; C07K 2319/035

(57) ABSTRACT

Compositions and methods are provided for genetically modifying cells to guide in situ chemical synthesis of electroactive, conductive, or insulating polymers on plasma membranes, organelle membranes, or subcellular surfaces of cells. In particular, compositions and methods are provided for genetically modifying excitable cells such as neurons, muscle cells, and endocrine cells to guide in situ chemical synthesis of polymers on the extracellular side of the plasma membrane. The subject methods can be used in various applications, for example, to assemble polymers in vivo at targeted locations to modulate electrical conduction and create new electrical conduction pathways, allow cell-type-specific neuromodulation, provide a conductive structure on cells for connection to electrodes, sensors, or other external electronic and electrochemical devices, and create a durable structure to replace damaged tissue for use in regenerative medicine.

### BACKGROUND OF THE INVENTION

The function of any complex biological system depends upon the actions and interactions of its cellular components. While physically isolating cells ex vivo for detailed individual manipulation has been immensely informative (for example in studying the nervous system) (Hodgkin et al. (1952) J. Physiol. 117, 500-544), the perturbation of cell types (Deisseroth (2015) Nat. Neurosci. 18, 1213-1225; Kim et al. (2017) Nat. Rev. Neurosci. 8, 222-235) or even individual cells (Prakash et al. (2012) Nat. Methods 9, 1171-1179; Rickgauer et al. (2014) Nat. Neurosci. 17, 1816-1824; Carrillo-Reid et al. (2016) Science 353, 691-694; Jennings et al. (2019) Nature 565, 645-649) in vivo has emerged as a complementary method to elucidate how essential behaviors of intact systems arise from the properties of components. Broad implementation of this cell-type targeting approach has created a wealth of experimental tools suitable for use in systems as complex as intact behaving metazoan animals, and has helped illuminate both basic science and human disease-related questions (Kim et al., supra). Moreover, as a result of this widespread tool development and adoption, new opportunities may be implicitly present, even if not yet appreciated, for distinct lines of investigation that could leverage the power of these cell-type targeting tools and approaches.

### SUMMARY OF THE INVENTION

Compositions, methods, and kits are provided for in situ polymer synthesis on cell membranes or subcellular components. In particular, compositions and methods are provided for genetically modifying cells to guide in situ chemical synthesis of electroactive, conductive, or insulating polymers. The subject methods utilize an enzyme that catalyzes polymerization of a desired polymer (i.e., polymerization enzyme) that is directed via a targeting sequence to the plasma membrane, organelle membranes, or subcellular surfaces of cells. Contacting cells with polymer precursors and a polymerization initiator results in in situ chemical synthesis of the polymer by the polymerization enzyme. In some embodiments, compositions and methods are provided for genetically modifying excitable cells such as neurons, muscle cells, and endocrine cells to guide in situ chemical synthesis of conductive polymers on the extracellular side of the plasma membrane. The subject methods can be used in various applications, for example, to assemble polymers in vivo at targeted locations to modulate electrical conduction and create new electrical conduction pathways, allow cell-type-specific neuromodulation, provide a conductive structure on cells for connection to electrodes, sensors, or other external electronic and electrochemical devices, and create a durable structure to replace damaged tissue for use in regenerative medicine.

In one aspect, a method for in situ polymer synthesis on a membrane or subcellular surface of a cell is provided, the method comprising: a) expressing a polymerization enzyme on the membrane or subcellular surface; and b) contacting the cell with one or more polymer precursors and a polymerization initiator, wherein the enzyme catalyzes polymerization of the polymer precursors resulting in production of the polymer on the membrane or the subcellular surface.

In certain embodiments, the membrane is a plasma membrane or an organelle membrane. In some embodiments, the polymerization enzyme is localized to an extracellular side of the plasma membrane. In some embodiments, the targeting peptide is an a-bungarotoxin (BTX) peptide comprising or consisting of the amino acid sequence of SEQ ID NO: 1 (MRYYESSLKSYPD).

In certain embodiments, the polymer is an electroactive polymer, a conductive polymer, or an insulating polymer. Exemplary conductive polymers include, without limitation, a polyaniline (PANI) polymer, a poly(3,4-ethylenedioxythiophene) (PEDOT) polymer, a 4-((5,7-di(thiophen-2-yl)-2,3-dihydrothieno[3,4-b][1,4]dioxin-2-yl)methoxy)butane-1-sulfonate (TETS) polymer, or a polyaniline-polyTETS (PANI-PTETS) polymer. The conductive polymer, PANI, can be produced from an aniline monomer and an aniline dimer (N-phenylenediamine). In some embodiments, the aniline monomer and the aniline dimer are at a 1:1 molar ratio. In certain embodiments, the polymers are doped, for example, by self-doping or adding an acid. For example, an acid-doped PANI polymer may be produced with p-toluenesulfonic acid (emeraldine salt of PANI). The conductive polymer, PEDOT, can be produced from EDOT monomers and further doped with polystyrene sulfonate (PEDOT:PSS) or self-doped. PANI-PTETS can be produced from polymerization of TETS with an aniline dimer and can be self-doped.

In certain embodiments, the cell is a live cell or a fixed cell. In certain embodiments, the cell is in a tissue, an organoid, or a subject. In some embodiments, the subject methods are performed on intact, living tissue. In some embodiments, the subject methods are performed on an invertebrate or vertebrate subject. In some embodiments, the subject is a model organism. In certain embodiments, the cell is a neuron (e.g., an inhibitory motor neuron or an excitatory motor neuron), muscle cell, or endocrine cell.

In certain embodiments, the polymerization enzyme is provided by a vector comprising a promoter operably linked to a polynucleotide encoding a fusion protein comprising the polymerization enzyme connected to a targeting peptide. In some embodiments, the targeting peptide localizes the polymerization enzyme to the plasma membrane or an organelle membrane. In some embodiments, the targeting peptide localizes the polymerization enzyme to the extracellular-facing surface of the plasma membrane. In some embodiments, the targeting peptide is an a-bungarotoxin (BTX) peptide comprising or consisting of the amino acid sequence of SEQ ID NO: 1. In some embodiments, the fusion protein further comprises a detectable label such as, including, without limitation, a fluorescent or bioluminescent protein. In some embodiments, the promoter is a human synapsin (hSyn) promoter. In some embodiments, the fusion protein further comprises a channelrhodopsin. In some embodiments, the vector is an adeno-associated virus (AAV) vector.

In certain embodiments, the polymerization enzyme is a peroxidase, and the polymerization initiator is a peroxide. In some embodiments, the peroxidase is an ascorbate peroxidase. For example, the ascorbate peroxidase may include, without limitation, an Apex2 ascorbate peroxidase such as humanized Apex2. In some embodiments, the peroxide is hydrogen peroxide (H₂O₂) or an organic hydroperoxide.

In another embodiment, a method for in situ polymer synthesis on a plasma membrane of a cell is provided, the method comprising: a) expressing a peroxidase on an extracellular-facing surface of the plasma membrane; and b) contacting the cell with one or more polymer precursors and a peroxide, wherein the peroxidase catalyzes production of the polymer on the extracellular-facing surface of the plasma membrane.

In certain embodiments, a kit is provided comprising one or more polymer precursors, hydrogen peroxide, and a vector comprising a promoter operably linked to a polynucleotide encoding a fusion protein comprising the peroxidase connected to a targeting peptide, wherein the targeting peptide localizes the peroxidase to the extracellular-facing surface of the plasma membrane. In some embodiments, the one or more polymer precursors in the kit comprise an aniline monomer and an aniline dimer (N-phenylenediamine). In some embodiments, the one or more polymer precursors in the kit comprise an aniline dimer (N-phenylenediamine) and TETS. In some embodiments, the kit further comprises p-toluenesulfonic acid. In some embodiments, the peroxidase included in the kit is an ascorbate peroxidase. For example, the kit may comprise an ascorbate peroxidase including, without limitation, an Apex2 ascorbate peroxidase such as humanized Apex2.

In another aspect, a method for in situ polymer synthesis in brain tissue is provided, the method comprising: a) expressing a polymerization enzyme on the plasma membranes of a plurality of neurons in the brain; and b) contacting the brain tissue with one or more polymer precursors and a polymerization initiator, wherein the enzyme catalyzes polymerization of the polymer precursors resulting in production of the polymer on the plasma membranes of a plurality of neurons in the brain.

In certain embodiments, the polymer is a conductive polymer. In some embodiments, the conductive polymer is a polyaniline (PANI) polymer, a poly(3,4-ethylenedioxythiophene) (PEDOT) polymer, a 4-((5,7-di(thiophen-2-yl)-2,3-dihydrothieno[3,4-b][1,4]dioxin-2yl)methoxy)butane-1-sulfonate (TETS) polymer, or a polyaniline-polyTETS (PANI-PTETS) polymer.

In certain embodiments, the polymerization enzyme is provided by a vector comprising a neuron-selective promoter (e.g., a human synapsin (hSyn) promoter operably linked to a polynucleotide encoding a fusion protein comprising the polymerization enzyme connected to a targeting peptide. In some embodiments, the targeting peptide is an a-bungarotoxin (BTX) peptide comprising or consisting of the amino acid sequence of SEQ ID NO: 1. In some embodiments, the fusion protein further comprises a channelrhodopsin. In some embodiments, the vector is an adeno-associated virus (AAV) vector.

### BRIEF DESCRIPTION OF THE DRAWINGS

FIGS. 1A-1G show genetically-targeted chemical assembly (GCA). (FIG. 1A) GCA schematic; specific instantiation shown is radical-triggered conductive polymerization in brain. Step I: Targeted cells (red) within the intact tissue express the custom enzyme, which is delivered to the extracellular membrane (red dots on cells in red dashed box) and thus the biocatalytic reaction center is delivered to the extracellular compartment while remaining connected to the targeted cell membrane (black dashed box). Biocompatible functional-material precursors (e.g. conductive polymer monomers, purple dots) are introduced, diffuse to targeted cells (purple dots in red and black dashed boxes), and are triggered to polymerize. Green neurons are non-enzyme-targeted neurons. Step II: Chemical assembly (via polymerization) of functional materials in vivo (purple). The non-enzyme-targeted neurons (green) don't have the functional materials assembled on their surface. (FIG. 1B) Engineering hApex2 peroxidase (top) with bungarotoxin (BTX) enables delivery of the reaction center (middle) to the extracellular space with a physical connection to the plasma membrane. Enhanced yellow fluorescence protein (eYFP) is fused for labelling. In some configurations, insertion of the ribosomal skip sequence p2A enables co-expression of a channelrhodopsin (ChR) with BTX-hApex2 (bottom). (FIGS. 1C-D) Implementation: schematics (FIG. 1C) and confocal fluorescence images (FIG. 1D) show anti-green fluorescence protein (GFP) and secondary antibody (conjugated with Cy5) staining in living neurons to label extracellular eYFP; note successful live extracellular expression demonstrated in central panel. White arrows in (FIG. 1D) highlight the extracellular labeled Cy5 signals. (FIG. 1E) Reaction schematic of hApex2-mediated polymerization from precursor reagents containing aniline monomer/dimer mixture. Labels 1-5 show chemical structures of N-phenylenediamine (aniline dimer, 1), aniline dimer radical cations (2), aniline monomer (3), aniline trimer radical cations (4) and polyaniline (PANI, 5). (FIG. 1F) Schematics show hApex2-mediated polymerization and deposition of PANI on the surface of targeted neurons. (FIG. 1G) In situ genetically-targeted synthesis and incorporation of conductive polymers. Epifluorescence images (eYFP channel) and transmitted bright-field (BF) images of cultured rat hippocampal neurons fixed by paraformaldehyde (PFA) and exposed to the PANI precursor reagent (1× PBS containing 0.5 mM aniline dimer, 0.5 mM aniline and 0.1 mM H₂O₂) for 30 min. Left: post reacted hSyn-eYFP neurons (labeled as hApex2(−)/PANI). Right: post reacted hSyn-BTX-hApex2-eYFP neurons (labeled as hApex2(+)/PANI). White arrow highlights the shell-like structure coated onto neurons after reaction.

FIGS. 2A-2S show chemical and electrical characterization of an in situ synthesized conductive polymer. (FIG. 2A) Molecular structures of PANI (red) conversion to doped PANI (dPANI, green), via acid (HX) treatment. (FIG. 2B) Normalized UV-vis spectra of 1) PANI (purple curve: ˜50,000 MW commercially sourced PANI was dissolved in chloroform and spin-coated on glass coverslip at 1000 r.p.m.), and 2) 4% PFA fixed, cultured rat hippocampal neurons on Matrigel-covered cover slip including hApex2(−) neurons (black curve), hApex2(−)/PANI neurons (blue curve) and hApex2(+)/PANI (red curve). Reaction conditions as in FIG. 1E. Red arrows indicate the maximum absorption peak. (FIG. 2C) Normalized UV-Vis spectra of hApex2(−)/PANI and hApex2(+)/PANI before and after p-toluenesulfonic acid treatment (0.1 mM, 15 min). Dashed red and green arrows highlight red-shift of the absorption peak in UV-vis spectra from ˜574 nm to ˜615 nm, and black arrow indicates enhanced absorption at ˜1000 nm for hApex2(+)/dPANI neurons. Inset: relative peak ratio at 1000 nm for hApex2(−)/dPANI and hApex2(+)/dPANI. The ratio is calculated based on the absorption value at 1000 nm vs absorption value at 1200 nm (baseline) before and after the acidic doping. Values are mean±s.e.m., n=4 for each condition, **** P<0.0001, unpaired, two-tailed t-test. (FIGS. 2D-2F) Averaged UV-vis spectra of PFA fixed, rat hippocampal neurons cultured on Matrigel coated glass cover slip reacted with polymer reagent (0.5 mM aniline, 0.5 mM aniline dimer and 0.1 mM H₂O₂) at different time course and doped by p-toluenesulfonic acid (0.1 mM, 15 min). n=4 for each condition. Red and green arrows highlight the increase of signature absorption peaks in hApex2(+)/PANI (FIG. 2E) and hApex2(−)/dPANI (FIG. 2F) samples. (FIGS. 2G-2I) Near edge X-ray absorption fine structure (NEXAFS) spectrum of fixed cultured neurons. Black, blue and red lines represent hApex2(+), hApex2(+)/PANI and hApex2(+)/dPANI neurons. Vertical dashed lines indicate peaks corresponding to signature bonds (insets) for different oxidation states of PANI. (FIGS. 2J-2O) Variable-pressure scanning electron microscopic (VPSEM) images of non-reacted, wild-type (J), hApex2(−)/PANI (FIG. 2K), hApex2(+)/PANI (FIG. 2L) and hApex2(+)/dPANI (FIG. 2M) neurons. Zoomed-in images of the blue dashed box (FIG. 2N) and red dashed box (FIG. 2O) regions from (FIG. 2M) show conductive polymer deposition on somatic and dendritic subcellular regions. Samples were not deposited with metal for enhanced contrast. (FIG. 2P) Schematic: building an electrical interface to fixed, post-reacted neurons (blue) with PANI (purple dashed lines) coated for conductivity measurements. Acid doping is specifically used to test presence of deposited conductive polymer (green dashed lines); to allow detection of long-range conduction pathways, 1-hr reaction was used at the same reagent concentrations from FIG. 1G. (FIG. 2Q) BF optical image of representative post-reacted neurons on the glass substrate measured with gold electrodes on the top for current-voltage (I-V) measurement. Green lines highlight individual projections from single neurons spanning multiple electrodes. Dashed box (left) highlighted the labeled neurons in right panel. (FIGS. 2R-2S) Representative I-V curves (FIG. 2R) and statistical summary of resistance changes (FIG. 2S, violin plots of logarithmic 10 scale of resistance, n=20 for each category, *** P<0.001, **** P<0.0001, unpaired, two-tailed t-test) between two electrodes formed on cultured hApex2(−) and hApex2(+) neurons on glass slides before and after acidic vapor treatment (HCl vapor). The reduction in hApex2(−)/dPANI sample is likely due to the ionic conductivity from the evaporated HCl solution.

FIGS. 3A-3J show electrophysiological characterization of in situ formed conductive polymers on living mammalian tissue. (FIG. 3A) Cell viability measured by live/dead cell kit assay (values are means±s.e.m., n (number of coverslips)=4 for hApex2(−) and 4 for hApex2(+) and cells for each coverslip>20, two-tailed t-test, * P<0.05). (FIG. 3B) Experiment scheme for slice physiology before- and after-reaction. (FIG. 3C) Photographic image of a brain slice after polymerization reaction within unilateral hippocampus after injection of virus encoding hApex2 (marked by a black arrow). The white dashed line highlighted the polymerized hippocampus. (FIGS. 3D-3H) Comparisons of cell input resistance (FIG. 3D), resting potential (FIG. 3E), membrane capacitance (FIG. 3F), electrochemical impedance at 1000 Hz (FIG. 3G) and holding current for −70 mV membrane potential (FIG. 3H), of hApex2(−) neurons (blue) and hApex2(+) neurons (red) pre- and post-polymerization. Note that membrane capacitance of hApex2(+) neurons show overall increasing trend after polymerization. (FIG. 3I) Representative voltage traces of hApex2(−) and hApex2(+) neurons pre- and post-polymerization stimulated by a continuous electrical pulse (1.5 seconds, 150 pA for hApex2(−) and 200 pA for hApex2(+) for the traces shown). (FIG. 3J) Comparisons of the number of spikes under continuous stimulation as in (I). Spike numbers are measured at the threshold current magnitude during the stepwise injection protocol as in FIG. 17. For (FIGS. 3D-3H) and (FIG. 3J), light color symbols represent values of individual neurons and dark color symbols represent values of means±s.e.m., * P<0.05, n=4 for hApex2(−) neurons and 5 for hApex2(+) neurons for FIGS. 3D-3H, and n=3 for hApex2(−) and 5 for hApex2(+) in (FIG. 3J), paired, two-tailed t-test.

FIGS. 4A-4S show in vivo cell-type-specific polymerization and behavioral remodeling in C. elegans. (FIG. 4A) Experiment scheme for the targeted polymerization to the pharyngeal muscle. (FIG. 4B) Bright-field (left) and fluorescence images of C. elegans expressing Pmyo-2:hApex2:mcd8:GFP (labeled as hApex2(+) as opposed to wild-type controls, which are labeled as hApex2(−) in this study). White arrow highlights the GFP labeled pharyngeal muscle in the C. elegans indicating the expression of hApex2. (FIG. 4C) Images of pharyngeal muscle of hApex2(−) /PANI and hApex2(+)/PANI C. elegans after 30-min reaction. Red arrows highlighted the significantly increased black particles in hApex2(+) C. elegans between pharyngeal muscle and epidermis. (FIGS. 4D-4E). Illustration (FIG. 4D) and comparisons (FIG. 4E) of the change of opacity for hApex2(−) and hApex2(+) C. elegans post-reaction. Signals were quantified through the ratio of changes in extracellular brightness to the intracellular brightness of pharyngeal muscle, (Values are means±s.e.m., n=3-4 animals, * P<0.05, two tailed, unpaired t-test). (FIGS. 4F-4G) Comparisons of pharyngeal pumping rate (per 20 seconds, FIG. 4F) and body bending rate (per minute, FIG. 4G) in hApex2(−)/PANI, hApex2(+)/H₂O₂ control, and Apex2(+)/polymerization C. elegans. (Values are means±s.e.m., * P<0.05, n=5 for all three conditions, two-tailed, unpaired, t-test) (FIG. 4H) Experiment scheme for cell-type-specific polymerization on GABAergic inhibitory motor neurons (Inhibitory→hApex2(+)) or cholinergic excitatory neurons (Excitatory→hApex2(+)) labelled worms. Black dashed lines represent the cell-type-specific polymerized polymers. (FIG. 4I) Fluorescence images of Inhibitory→hApex2(+) neurons (top) and Excitatory→hApex2(+) neurons (bottom) shows GABAnergic inhibitory motor neurons and cholinergic excitatory motor neurons expressing hApex2:mcd8:GFP under Punc-47 and Punc-17 promoters, respectively. (FIG. 4J) Excitatory→hApex2(+) neurons show significantly reduced locomotion after polymerization, whereas Inhibitory→hApex2(+) strains show negligible difference. (n=30 for hApex2(−), 26 for Inhibitory→hApex2(+), and 32 for Excitatory→hApex2(+), *** P<0.001, **** P<0.0001, one-sided Fisher's exact tests). (FIG. 4K) Summary of aldicarb resistance assay for C. elegans after polymerization (n=4 experiments, with 25 animals per strain in each experiment, one-way ANOVA test, with Tukey correction, * P<0.05, ** P<0.01). (FIGS. 4L-4M) Representative bright-field images of Apex2(−) /PANI and inhibitory→hApex2(+)/PANI worms (FIG. 4L) and their traces (FIG. 4M) after polymerization. Black arrows indicate where the worms took reversals. (FIG. 4N) Summary of number of reversals for C. elegans during their movement. (FIG. 4O) The percentage of sharp bends (<90° bends) in total bends for C. elegans during their movement. (For (FIGS. 4N-4O), values are means±s.e.m., * P<0.05, ** P<0.01, n=10 for all samples, two-tailed, unpaired t-test). (FIG. 4P) Summary of amplitude of C. elegans movement. (inset shows scheme of how to calculate the amplitude). (FIG. 4Q) Summary of wavelength of C. elegans movement (inset shows the scheme of how to calculate the wavelength). (FIGS. 4R-4S) unc-25 null mutation control for the behavior of C. elegans. (FIG. 4R) Summary of wavelength of C. elegans movement. (FIG. 4S) Summary of amplitude of C. elegans movement. **** P<0.001, n=10 animals for each condition, two-tailed, unpaired, t-test. The calculation methods for amplitude and wavelength are the same as those in (FIGS. 4P-4Q).

FIGS. 5A-5C show control conditions for verifying extracellular expressions of hApex2. (FIG. 5A) Engineered hApex2 peroxidase fused with enhanced yellow fluorescence protein (eYFP, top) and glycosyl phosphatidyl inositol (GPI, bottom). (FIGS. 5B-5C) Implementation: schematics (FIG. 5B) and confocal fluorescence images (FIG. 5C) show anti-green fluorescence protein (GFP) and secondary antibody (conjugated with Cy5) staining in living neurons to label extracellular eYFP. Note while GPI has been reported (5) to be membrane bound, the expected localization would be internal not external, and indeed our experiment result shows that the GPI motif did not drive hApex2 to the extracellular side.

FIGS. 6A-6C show control conditions demonstrating that the aniline and aniline-dimer mixed solution is crucial for in situ genetically-targeted polymerization. (FIG. 6A) Epifluorescence images (eYFP channel) and transmission bright-field (BF) of cultured rat hippocampal neurons fixed by paraformaldehyde (PFA) and exposed to the PANI precursor reagent (1× PBS containing 1 mM aniline and 0.1 mM H₂O₂) for 30 min. Top row: hApex2(+)/PANI neurons. Bottom row: hApex2(−) /PANI neurons. (FIG. 6B) Epifluorescence images (eYFP channel), transmission bright-field (BF) of cultured rat hippocampal neurons fixed by paraformaldehyde (PFA) and exposed to the PANI precursor reagent (1× PBS containing 1 mM aniline dimer and 0.1 mM H₂O₂) for 30 min. Top row: hApex2(+)/PANI neurons. Bottom row: hApex2(−)/PANI neurons. (FIG. 6C) Summary of opacity difference between neurons and background (Dopacity) to the background opacity. Values are means±s.e.m., *** P<0.001, **** P<0.0001, n=5 for all three conditions, two-tailed, unpaired, t-test.

FIGS. 7A-7C show a time course of the reaction showing an increase of PANI with increased reaction time. (FIGS. 7A-7B) Optical photographic images show the change of color for hApex2(−) (FIG. 7A) and hApex2(+) (FIG. 7B) rat hippocampal neurons cultured on Matrigel coated glass cover slip reacted with polymer reagent (0.5 mM aniline, 0.5 mM aniline dimer and 0.1 mM H₂O₂). (FIG. 7C) hApex2(+)/PANI neurons were doped by p-toluenesulfonic acid (0.1 mM, 15 min). The purple color indicates PANI and green color indicates doped PANI.

FIGS. 8A-8D show spectroscopic characterization of in situ, genetically-targeted polymers formed on cells confirming chemical structure of PANI and p-toluenesulfonic acid doping. (FIG. 8A) X-ray photoelectron spectroscopy (XPS) S 2p core level spectrum of fixed, cultured neurons with PANI reaction before and after acidic acid doping. The reaction condition for hApex2(−)/PANI and hApex2(+)/PANI neurons is 0.1 mM H₂O₂, 0.5 mM aniline and 0.5 mM aniline dimer for 30 min. The doping condition for hApex2(−)/dPANI and hApex2(+)/dPANI is 0.1 mM p-toluenesulfonic acid for 15 min. (FIGS. 8B-8C) Near edge X-ray absorption fine structure (NEXAFS) spectrum characterization of fixed, cultured hApex2(+) neurons with PANI reaction and doping. The reaction condition is 0.1 mM H₂O₂, 0.5 mM aniline and 0.5 mM aniline dimer for 30 min. The doping condition is 0.1 mM p-toluenesulfonic acid for 15 min. The samples were further washed by DI water for 3 times to remove any residue reagents. Black boxes highlighted the zoomed-in regions in FIG. 2G-I. (FIG. 8B) C K-edge NEXAFS spectra of hApex2(+), hApex2(+)/PANI and hApex2(+)/dPANI neurons. (FIG. 8C) N K-edge NEXAFS spectra of hApex2(+), hApex2(+)/PANI and hApex2(+)/dPANI neurons. (FIG. 8D) Photo-electron spectroscopy in air (PESA) characterization of hApex2(−)/PANI, hApex2(+)/PANI and hApex2(+)/dPANI fixed, cultured neurons on glass slides. The reaction condition is 0.1 mM H₂O₂, 0.5 mM aniline and 0.5 mM aniline dimer for 30 min. The doping condition is 0.1 mM p-toluenesulfonic acid for 15 min. The emission power was increased 20 times for hApex2(−)/PANI sample compared to that for hApex2(+)/PANI and hApex2(+)/dPANI samples to get the detectable signal.

FIGS. 9A-9B show variable-pressure scanning electron microscopic (VP-SEM) images showing the enhanced surface conductivity of neurons after polymerization. VPSEM images of cultured hippocampal hApex2(−) neurons (FIG. 9A) and hApex2(+) neurons (FIG. 9B) prior and post to polymerization and acidic doping. The culture neurons were on the Matrigel coated glass slides. The reaction condition is 0.1 mM H₂O₂, 0.5 mM aniline and 0.5 mM aniline dimer for 30 min. The doping condition is 0.1 mM p-toluenesulfonic acid for 15 min. Samples were immersed in deionized (DI) water for imaging. White arrows highlight the soma of neurons.

FIGS. 10A-10C show conductivity characterizations of polymers coated on a sheet of HEK293 cells showing increased conductivity. (FIG. 10A) BF optical image of representative post-reacted HEK cells on the glass substrate measured with gold electrodes on the top for current-voltage (I-V) measurement. (FIGS. 10B-10C) Representative I-V curves (FIG. 10B) and statistical summary of resistance changes (FIG. 10C, values are mean±s.e.m, n=10 for each category, ** P<0.01, **** P<0.0001, unpaired, two-tailed t-test) between two electrodes formed on cultured hApex2(−) and hApex2(+) HEK on glass slides before and after reaction and acidic vapor treatment (HCl vapor).The reaction condition is 0.1 mM H₂O₂, 0.5 mM aniline and 0.5 mM aniline dimer for 1 hr. HCl vapor is used to dope samples.

FIGS. 11A-11D show self-doping monomer (FIG. 11A) Scheme for synthesis of sodium 4-((5,7-di(thiophen-2-yl)-2,3-dihydrothieno[3,4-b][1,4]dioxin-2-yl)methoxy)butane-1-sulfonate (termed as TETs). (FIG. 11B) Confocal fluorescence images (eYFP channel) and transmitted bright-field (BF images of cultured rat hippocampal neurons fixed by paraformaldehyde (PFA) and exposed to the PANI-PTETs precursor reagent (1× PBS containing 0.5 mM aniline dimer, 0.5 mM TETs and 0.1 mM H₂O₂) for 30 min. (FIG. 11C) Summary of opacity difference between neurons and background (Dopacity) to the background opacity. Values are means±s.e.m., **** P<0.0001, n=5 for both conditions, two-tailed, unpaired, t-test. (FIG. 11D) XPS S 2p core level spectrum of fixed, cultured neurons with PANI and PTETs reaction confirming the incorporation of PTETs.

FIGS. 12A-12C show conductivity characterizations of self-doping polymers coated on a sheet of HEK293 cells showing intrinsic detectable conductive pathway. (FIG. 12A) BF optical image of representative post-reacted HEK cells on the glass substrate measured with gold electrodes on the top for current-voltage (I-V) measurement. (FIGS. 12B-12C) Representative I-V curves (FIG. 12B) and statistical summary of resistance changes (FIG. 12C, values are mean±s.e.m, n=10 for each category, **** P<0.0001, unpaired, two-tailed t-test) between two electrodes formed on cultured hApex2(−) and hApex2(+) HEK on glass slides before and after reaction. The reaction condition is 0.1 mM H₂O₂, 0.5 mM TETs and 0.5 mM aniline dimer for 1 hr. Samples are washed by DI water and air-dried for the measurement.

FIGS. 13A-13E show in situ, genetically-targeted polymerization in stem cell-derived human cortical spheroids (hCS) shows the intact-tissue polymerization of CPs. (FIG. 13A) Optical photographic images of representative Syn-eYFP (labelled as hApex2(−)) hCS (top) and Syn-BTX-hApex2− eYFP (labelled as hApex2(+)) hCS (bottom) pre and post PANI reaction. hCS were reacted at 0.1 mM H₂O₂, 0.5 mM aniline and 0.5 mM aniline dimer for 30 min. (FIG. 13B) Confocal fluorescence and BF images of Apex2(−)/PANI hCS (top) and hApex2(+)/PANI (bottom) hCS. (FIGS. 13C-13D) Zoomed-in confocal images of dashed boxes highlighted region in (FIG. 13B). White arrows highlight the shell-like polymer structures on hApex2(+) neurons in hCS. (FIG. 13E) Summary of opacity difference between neurons and background (Dopacity) to the background opacity. Values are means±s.e.m., **** P<0.0001, n=5 cells from one hCS for both conditions, two-tailed, unpaired, t-test.

FIGS. 14A-14B show living neuron reactions and images demonstrating 10 min reaction with H₂O₂ concentration at 0.05 mM can reliably generate PANI on neurons. (FIG. 14A) Representative images of alive, cultured rat hippocampal hApex2(+) neurons after PANI reaction. Neurons were exposed to reagents with 0.05 mM H₂O₂, 0.5 mM aniline and 0.5 mM aniline dimer for 0, 10 and 30 min before imaging. (FIG. 14B) Statistic summary of number of particles per area on neurons after reaction. (values are mean±s.e.m., ** P<0.01, *** P<0.0001, unpaired, two-tailed t-test).

FIGS. 15A-15C show UV-vis characterization of in situ, genetically targeted formed PANI on living, neurons demonstrating 10 min reaction with H₂O₂ concentration at 0.05 mM can reliably generate PANI on neurons. (FIGS. 15A-15B) Normalized UV-vis spectra of hApex(−) neurons (FIG. 15A) and hApex2(+) neurons (FIG. 15B). (FIG. 15C) Statistic summary of the relative peak ratio of (574 nm vs. 1200 nm). Values are mean±s.e.m., * P<0.05, *** P<0.001, **** P<0.0001, unpaired, two-tailed t-test.

FIGS. 16A-16D show electrophysiological characterization of cultured neurons after GCA (FIG. 16A) Left—representative action potential spikes elicited by current injection (500 pA, 10 ms) with-(black) and without-(purple) the PANI polymerization reaction. Similar results were observed in five independent experiments. Right—magnified view of overlaid spikes as in left (orange box) for hApex2(−) and hApex2(+) neurons. (FIG. 16B) Comparison of hApex2(−) and hApex2(+) cultured rat hippocampal neurons pre- and post-polymerization reaction for input resistance and resting potential (means±s.e.m., n=5 for all samples, two-tailed unpaired t-test, N.S.=not significant). (FIG. 16C) Left—example traces of capacitance currents measured in pre-(black) and post-(purple) polymerized neurons after 10 mV hyperpolarization. Right—comparisons of membrane capacitance of hApex2(−) (blue) and hApex2(+) (red) neurons with (+) and without (−) polymer coating. Note increased capacitance in hApex2(+) neurons with reaction (FIG. 16D) Comparisons of spike width (left), spike latency (middle) and spike amplitude (right) of hApex2(−) (blue) and hApex2(+) (red) with (+) and without (−) polymer coating. (For FIGS. 16B-16D, values are means±s.e.m., ** P<0.01, n=10 for hApex2(−) and hApex2(+) in FIG. 16C, and n=5 for the rest).

FIGS. 17A-17F show electrophysiological characterization of living brain slice after GCA (FIGS. 17A-17D) Comparison of hApex2(−) and hApex2(+) neurons in brain slice pre- and post-polymerization for spike width (FIG. 17A), spike latency (FIG. 17B) spike amplitude (FIG. 17C) and current injection threshold for action potential generation (FIG. 17D). Action potentials for FIG. 17A-17D were induced through either pulsed current injection (100-500 pA, 5 ms, 5 Hz, for FIGS. 17A-17C) or stepwise current injection (−100-400 pA, 1.5 second, for FIG. 17D) while holding the membrane potential at −70 mV. Input resistance was calculated after −10 mV hyperpolarization at the voltage clamp mode. For detailed explanation of estimation of these parameters, see Methods. (Light color symbols represent individual sample. Dark color symbols represent mean±s.e.m. n=4 for hApex2(−) and 5 for hApex2(+) neurons, two-tailed unpaired t-test). (FIG. 17E) Representative entire traces of action potential generation elicited by stepwise current injection (from −100 pA to 400 pA) in hApex2(+) and hApex2(−) neurons before polymerization reaction. (FIG. 17F) Comparison of spiking frequencies between hApex2(−) and hApex2(+) neurons before polymerization as in (FIG. 17E) Note there is no significant difference between two populations (means±s.e.m., n=3 for hApex2(−) and 5 for hApex2(+) neurons, two-tailed unpaired t-test, N.S.=not significant).

FIGS. 18A-18B show detailed explanation of aldicarb resistance assays using unc-10(o) and syd-2(wy5) mutants as positive controls. Summary of aldicarb resistance assay for C. elegans after polymerization with unc-10 (md1117) mutation (strongly resistant) as positive control (FIG. 18A) and syd-2 mutation (weakly resistant) as positive control (FIG. 18B) (For both panels, n=2 experiments with 25 animals per strain per experiment). For the experiment, 25 worms of each were placed on plates containing 0.7 mM aldicarb and assayed for acute paralysis, defined as lacking movement of the body after being prodded three times on the head and tail. hApex2(−) and hApex(+) strains were assayed in four experiments, and each mutant was used in two of those experiments. Note that in both experiments, although different positive controls were used, all three control and experimental groups show identical patterns of resistance to aldicarb.

### DETAILED DESCRIPTION OF THE INVENTION

Compositions, methods, and kits are provided for in situ polymer synthesis on cell membranes or subcellular components. In particular, compositions and methods are provided for genetically modifying cells to guide in situ chemical synthesis of electroactive, conductive, or insulating polymers on plasma membranes, organelle membranes, or subcellular surfaces of cells.

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the vector” includes reference to one or more vectors and equivalents thereof, e.g. recombinant polynucleotides or constructs known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

As used herein, a “cell” refers to any type of cell from a prokaryotic, eukaryotic, or archaeon organism, including bacteria, archaea, fungi, protists, plants, and animals, including cells from tissues, organs, organoids, and biopsies, as well as recombinant cells, cells from cell lines cultured in vitro, and cellular fragments, cell components, or organelles. The term also encompasses artificial cells, such as nanoparticles, liposomes, polymersomes, or microcapsules encapsulating nucleic acids. A cell may include a fixed cell or a live cell. The methods described herein can be performed, for example, on a sample comprising a single cell or a population of cells or on a tissue, organ, organoid, or subject.

A “live cell”, as used herein, refers to an intact cell, naturally occurring or modified. The live cell may be isolated from other cells, mixed with other cells in a culture, or within a tissue (partial or intact) or an organism. In some embodiments, the live cell is a cell engineered to express a polymerization enzyme (e.g., peroxidase) as described herein. In some embodiments, the live cell expresses an enzyme that catalyzes polymerization of a polymer that is targeted to a membrane or a subcellular surface of a cell, for example, via a localization signal or targeting peptide within or fused to the polymerization enzyme.

“Biocompatible” refers to a material that is non-toxic to a cell or subject.

The term “conductive polymer” refers to a polymer that conducts electricity.

The term “polymerization enzyme” refers to an enzyme that catalyzes a reaction that results in polymerization of polymer precursors in the presence of a polymerization initiator.

The terms “nucleic acid,” “nucleic acid molecule,” “polynucleotide,” and “oligonucleotide” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. There is no intended distinction in length between the terms “nucleic acid,” “nucleic acid molecule,” “polynucleotide,” and “oligonucleotide” and these terms will be used interchangeably.

The terms “protein,” “polypeptide,” and “peptide” refer to any compound comprising naturally occurring or synthetic amino acid polymers or amino acid-like molecules including but not limited to compounds comprising amino and/or imino molecules. No particular size is implied by use of the terms “protein,” “polypeptide,” and “peptide,” and these terms are used interchangeably.

As used herein, the term “binding pair” refers to first and second molecules that specifically bind to each other, such as a ligand and a receptor, an antigen and an antibody, or biotin and streptavidin. “Specific binding” of the first member of the binding pair to the second member of the binding pair in a sample is evidenced by the binding of the first member to the second member, or vice versa, with greater affinity and specificity than to other components in the sample. The binding between the members of the binding pair is typically noncovalent.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The terms “fusion protein,” “fusion polypeptide,” or “fusion peptide” as used herein refer to a fusion comprising a polymerization enzyme (e.g., peroxidase) in combination with a targeting peptide that localizes the polymerization enzyme to a membrane (e.g., plasma membrane or organelle membrane) or subcellular surface of a cell as part of a single continuous chain of amino acids, which chain does not occur in nature. The polymerization enzyme and the targeting peptide may be connected directly to each other by peptide bonds or may be separated by intervening amino acid sequences. The targeting peptide may be, for example, a signal peptide or a peptide that binds to a membrane protein. The fusion protein may also contain other sequences such as a detectable label (e.g., a fluorescent or bioluminescent protein) or tag sequences.

By “fragment” is intended a molecule consisting of only a part of the intact full-length sequence and structure. The fragment can include a C-terminal deletion an N-terminal deletion, and/or an internal deletion of the polypeptide. Active fragments of a particular protein or polypeptide will generally include at least about 5-14 contiguous amino acid residues of the full length molecule, but may include at least about 15-25 contiguous amino acid residues of the full-length molecule, and can include at least about 20-50 or more contiguous amino acid residues of the full-length molecule, or any integer between 5 amino acids and the full-length sequence, provided that the fragment in question retains biological activity.

“Substantially purified” generally refers to isolation of a substance (compound, polynucleotide, protein, polypeptide, peptide composition) such that the substance comprises the majority percent of the sample in which it resides. Typically, in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a protein, polypeptide or peptide, that the indicated molecule is separate and discrete from the whole organism with which the molecule is found in nature or is present in the substantial absence of other biological macro molecules of the same type. The term “isolated” with respect to a nucleic acid is a nucleic acid molecule devoid, in whole or part, of sequences normally associated with it in nature; or a sequence, as it exists in nature, but having heterologous sequences in association therewith; or a molecule disassociated from the chromosome.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells,” “host cells,” “cells”, “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single stranded DNA (e.g., a M13 origin of replication), at least one multiple cloning site, and a “mammalian” origin of replication (e.g., a SV40 or adenovirus origin of replication).

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, plasmid vectors, viral vectors (e.g., adeno-associated virus vector), or non-viral vectors.

The term “subject” or “host subject” includes bacteria, archaea, fungi, protists, plants, and animals (both vertebrates and invertebrates), including, without limitation, plants such as flowering plants (e.g., Arabidopsis thaliana), conifers and other gymnosperms, ferns, clubmosses, hornworts, liverworts, mosses (e.g., Physcomitrella patens), and green algae (e.g., Chlamydomonas reinhardtii); fungi such as molds and yeasts (e.g., Saccharomyces cerevisiae, Schizosaccharomyces pombe), protists such as amoebae, flagellates, and ciliates (e.g., Tetrahymena thermophila); worms (e.g., Caenorhabditis elegans), insects such as beetles, ants, bees, moths, butterflies, and flies (e.g., Drosophila melanogaster), amphibians such as frogs (e.g., Xenopus tropicalis, Xenopus laevis) and salamanders (e.g., axolotls); fish (e.g., Danio rerio, Fundulus heteroclitus, Nothobranchius furzen); reptiles; mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, and geese. In some cases, the methods of the invention find use in experimental animals, in veterinary application, and in the development of animal models for disease, including, but not limited to, rodents including mice, rats, and hamsters; primates, and transgenic animals.

In some embodiments, the subject is a “model organism”, such as an animal model or test subject for use in scientific or biomedical research or drug screening. Model organisms include, but are not limited to, invertebrates such as worms (e.g., Caenorhabditis elegans) and flies (e.g., Drosophila melanogaster); amphibians such as frogs (e.g., Xenopus tropicalis, Xenopus laevis) and salamanders (e.g., axolotls); fish (e.g., Danio rerio, Fundulus heteroclitus, Nothobranchius furzen), mammals such as rodents, including guinea pigs (e.g., Cavia porcellus), mice (e.g., Mus musculus), and rats (e.g., Rattus norvegicus), and non-human primates such as the rhesus macaque and chimpanzee.

The term “animal” is used herein to include all vertebrate and invertebrate animals, except humans. The term also includes animals at all stages of development, including embryonic and fetal stages.

A “transgenic organism” is an organism containing one or more cells bearing genetic material received, directly or indirectly, by deliberate genetic manipulation at a subcellular level, such as by microinjection or infection with recombinant virus. An introduced DNA molecule may be integrated within a chromosome, or it may be extra-chromosomally replicating DNA. If a DNA molecule is introduced into a germ line cell, the genetic material containing the DNA molecule may be transferred to offspring. An offspring possessing some or all of that genetic material is also considered to be a transgenic organism.

### In Situ Polymer Synthesis

Compositions and methods are provided for genetically modifying a cell to guide in situ chemical synthesis of a polymer on a membrane or subcellular surface. The subject methods utilize an enzyme that catalyzes polymerization of a desired polymer (i.e., a polymerization enzyme), which is directed via a targeting sequence to a particular cellular location such as the plasma membrane, an organelle membrane, or subcellular surface of a cell. Contacting cells with polymer precursors and a polymerization initiator results in in situ chemical synthesis of the polymer by the polymerization enzyme.

The methods may be applied to a single cell or population of cells of interest, or a tissue, organ, or organoid. Although the methods for in situ polymer synthesis and the related reagents, materials and compositions described herein are well suited for use in live cells and tissues, it should be appreciated that their use is not so limited, but that they can also be applied to fixed cells and tissues, for example, fixed cells and tissues obtained from a subject, e.g., in a clinical or research setting. The methods may also be applied to live subjects, including, without limitation, model organisms, laboratory research animals, and transgenic animals.

In general, the methods and strategies for in situ polymer synthesis employ a polymerization enzyme, which catalyzes a radical initiated polymerization step. In some embodiments, the polymerization enzyme is a peroxidase. Peroxidases catalyze the reaction of a peroxide (e.g., hydrogen peroxide (H₂O₂) or organic hydroperoxide) with an electron donor to generate short-lived reactive free radicals that initiate polymerization of polymer precursors. For example, in situ polymer synthesis can be performed in the presence of hydrogen peroxide and polymer precursors, wherein the peroxidase catalyzes the polymerization of the polymer precursors under physiological conditions. Exemplary peroxidases suitable for use as polymerization enzymes include horseradish peroxidase, soybean peroxidase, and ascorbate peroxidase. In certain embodiments, the polymerization enzyme is an engineered ascorbate peroxidase (e.g., APEX or APEX2). An advantage of using certain engineered ascorbate peroxidases is they can be expressed and active in a reducing cellular environment. For a description of APEX and APEX2 engineered ascorbate peroxidases, see. e.g., Martell et al. (2012) Nat. Biotechnol. 30:1143-1148, Lam et al. (2015) Nat. Methods 12:51-54, and U.S. Pat. No. 9,624,524; herein incorporated by reference in their entireties.

Peroxidases can be used, for example, to catalyze production of conductive polymers including, without limitation, polyaniline (PANI), poly(3,4-ethylenedioxythiophene) (PEDOT), 4-((5,7-di(thiophen-2-yl)-2,3-dihydrothieno[3,4-b][1,4]dioxin-2-yl)methoxy)butane-1-sulfonate (TETS), and polyaniline-polyTETS (PANI-PTETS). The conductive polymer, PANI, can be produced from an aniline monomer and an aniline dimer (N-phenylenediamine). In some embodiments, the aniline monomer and the aniline dimer are at a 1:1 molar ratio. In certain embodiments, the polymers are doped, for example, by self-doping or adding an acid. For example, an acid-doped PANI polymer may be produced with p-toluenesulfonic acid (emeraldine salt of PANI). The conductive polymer, PEDOT, can be produced from EDOT monomers and further doped with polystyrene sulfonate (PEDOT:PSS) or self-doped. PANI-PTETS can be produced from polymerization of TETS with an aniline dimer and can be self-doped.

The cell is contacted with polymer precursors and a polymerization initiator under conditions suitable for the polymerization enzyme to generate the polymer in situ at a target site where the polymerization enzyme is expressed. The polymerization enzyme may be delivered to the cell interior or exterior, depending on where in situ synthesis of the polymer is desired. In some embodiments, the polymerization enzyme is delivered to the plasma membrane, and in some instances to the extracellular side of the plasma membrane. In other embodiments, the polymerization enzyme is delivered to the interior of the cell, and in some instances, to specific subcellular compartments. In some embodiments, the polymerization enzyme is delivered to a tissue followed by perfusion of the tissue with polymer precursors and a polymerization initiator capable of rapid diffusion through the intact tissue.

A cell may be genetically modified to express the polymerization enzyme at a target site where production of the polymer is desired. The polymerization enzyme may be introduced into a cell by transfecting the cell with a recombinant polynucleotide comprising a promoter operably linked to a polynucleotide encoding the polymerization enzyme. The recombinant polynucleotide may comprise an expression vector, for example, a bacterial plasmid vector or a viral expression vector, such as, but not limited to, an adeno-associated virus (AAV), adenovirus, retrovirus (e.g., γ-retrovirus and lentivirus), poxvirus, baculovirus, or herpes simplex virus vector, as described further below.

In some embodiments, the polymerization enzyme is engineered to improve its capability in in situ polymer synthesis. For example, the polymerization enzyme can be engineered to be expressed only on a cell membrane (e.g., plasma membrane or organelle membrane) or subcellular surface or within a subcellular compartment or structure of interest. The polymerization enzyme may also be engineered to comprise one or more mutations that enhance its catalytic activity on a membrane or subcellular surface or within a subcellular compartment or structure of interest.

The polymerization enzyme can be directed to a cell membrane, cellular compartment, or specific protein of interest in a number of ways. For example, the polymerization enzyme may be modified to include a targeting sequence that directs the polymerization enzyme to the subcellular region of interest. Targeting sequences that can be used include, but are not limited to, a secretory protein signal sequence, a membrane protein signal sequence, a nuclear localization sequence, a mitochondrial localization sequence, an outer mitochondrial membrane sequence, an endoplasmic reticulum localization sequence, an endoplasmic reticulum membrane targeting sequence, a nucleolar localization signal sequence, a nuclear export signal sequence, a peroxisome localization sequence, and a protein binding motif sequence. Examples of targeting sequences include those targeting the plasma membrane (e.g., a-bungarotoxin (BTX) motif (SEQ ID NO:1) targeting the extracellular side of the plasma membrane, signal peptides directing post-translational modification with a glycosylphosphatidylinositol (GPI) anchor, sequences directing prenylation such as the CaaX motif, where “a” is an aliphatic amino acid, CC, CXC, or CCXX at the C-terminus, and membrane spanning sequences), synapses (e.g., S/TDV or fusion to GAP 43, kinesin or tau), or protein-protein interaction motifs (e.g., SH2, SH3, PDZ, WW, RGD, Src homology domain, DNA-binding domain, and SLiMs).

In other embodiments, the polymerization enzyme is covalently linked to a peptide or protein that directs the polymerization enzyme to a subcellular region of interest, such as a membrane protein, a cytosolic protein, a nuclear protein, a mitochondrial protein, a P-body protein, or a secretory pathway protein. Attachment of the polymerization enzyme to the protein of interest results in in situ polymer synthesis in the vicinity of the protein of interest in the locations where the protein resides in the cell. Alternatively, the polymerization enzyme can be covalently linked to an antibody that specifically binds to a particular epitope found on certain proteins in a subcellular region of interest, which similarly allows in situ polymer synthesis in the vicinity of the epitope in the subcellular region of interest.

Polymerization enzymes can be genetically targeted to cellular regions in cell-types of interest such that newly synthesized polymers are deposited onto targeted cells at subcellular locations (e.g., the plasma membrane, extracellular side of the plasma membrane, the nucleus, endoplasmic reticulum, Golgi, mitochondria, mitochondria outer membrane, mitochondria inner membrane, mitochondria matrix space, chloroplasts, synaptic cleft, presynaptic membrane, postsynaptic membrane, dendritic spines, transport vesicles, regions of contact between mitochondria and endoplasmic reticulum, nuclear membrane, etc.).

In some embodiments, a polymerization enzyme is engineered to be expressed and/or targeted in vivo or in situ to specific cells (e.g., neurons, muscle cells), cellular compartments (e.g., endoplasmic reticulum, Golgi apparatus, mitochondria, nucleus, the synaptic cleft, transport vesicles, etc.), and/or macromolecular complexes (e.g., protein complexes such as ribosomes, nuclear pore complex, fatty acid synthases) of interest. In some embodiments, a cell-type or tissue-selective promoter is used to localize expression of the polymerization enzyme. For example, to target neurons, a neuron-selective promoter may be used to selectively express the polymerization enzyme in neurons. Exemplary neuron-selective promoters include, without limitation, the synapsin promoter, α-CaMKII promoter fugu promoters, engineered promoters comprising a neuron-restrictive silencer element (NRSE), and h12R-tdTomato and h56D-tdTomato promoters. To target muscle cells, a muscle-selective promoter may be used to selectively express the polymerization enzyme in muscle cells. In some embodiments, the promoter may be selective for skeletal muscle, smooth muscle, or cardiac muscle cells. Exemplary muscle-selective promoters that can be used include, without limitation, the muscle creatine kinase (MCK) promoter, troponin I promoter, and C5-12 promoter.

### Cells and Tissue

In certain embodiments, the methods are applied to a population of cells, which may include a plurality of cell types including, but not limited to, excitatory neurons, inhibitory neurons, and non-neuronal cells. In situ polymerization may be performed, for example, on cells of an organism, a single cell type derived from an organism, or can be a mixture of cell types. Included are naturally occurring cells and cell populations, genetically engineered cell lines, cells derived from transgenic animals, etc. Virtually any cell type and size can be accommodated. Suitable cells include bacterial, fungal, plant and animal cells. In one embodiment, the cells are mammalian cells, e.g. complex cell populations such as occurring naturally in tissues, for example, neural tissue, blood, liver, pancreas, bone marrow, skin, and the like. In some embodiments, the tissue is intact live tissue. In other embodiments, the tissue is disrupted into a monodisperse suspension. Alternatively, the cells may be a cultured population, e.g. a culture derived from a complex population, a culture derived from a single cell type where the cells have differentiated into multiple lineages, or where the cells are responding differentially to stimulus, and the like.

Cell types that can find use in the subject methods include cells involved with particular tissues or organs, and genetically modified cells thereof. For example, cell types of interest in the nervous system, including the spinal cord and brain, include, without limitation, neurons, glia, astrocytes, dendrocytes, etc. Cells may also include stem and progenitor cells, e.g. embryonic stem cells, hematopoietic stem cells, mesenchymal stem cells, neural crest cells, muscle cells, smooth and skeletal muscle cells, cardiomyocytes, mesenchymal cells, endothelial cells, epithelial cells, adipocytes, endocrine cells, hematopoietic cells, such as lymphocytes, including T-cells, such as Th1 T cells, Th2 T cells, Th17 T cells, ThO T cells, cytotoxic T cells, regulatory T cells; B cells, pre-B cells, etc.; monocytes; dendritic cells; neutrophils; and macrophages; natural killer cells; mast cells, etc. Cells may be non-adherent, e.g. blood cells including monocytes, T cells, B-cells; tumor cells, etc., or adherent cells, e.g. epithelial cells, endothelial cells, neural cells, etc.

The cells may also be transformed or neoplastic cells of different types, e.g. carcinomas of different cell origins, lymphomas of different cell types, etc. The American Type Culture Collection (Manassas, Va.) has collected and makes available over 4,000 cell lines from over 150 different species, over 950 cancer cell lines including 700 human cancer cell lines. The National Cancer Institute has compiled clinical, biochemical and molecular data from a large panel of human tumor cell lines, these are available from ATCC or the NCI (Phelps et al. (1996) Journal of Cellular Biochemistry Supplement 24:32-91). Included are different cell lines derived spontaneously, or selected for desired growth or response characteristics from an individual cell line; and may include multiple cell lines derived from a similar tumor type but from distinct patients or sites.

Such cells can be acquired from an individual using, e.g., a draw, a lavage, a wash, surgical dissection etc., from a variety of tissues, e.g., blood, marrow, a solid tissue (e.g., a solid tumor), ascites, by a variety of techniques that are known in the art. Cells may be obtained from fixed or unfixed, fresh or frozen, whole or disaggregated samples. Disaggregation of tissue may occur either mechanically or enzymatically using known techniques.

In particular, the subject methods are applicable to investigation of neurons and neural tissue. Polymers may be deposited in situ on a membrane (e.g., extracellular side of the plasma membrane) or subcellular surface or structure of interest of any type of neuron including, without limitation, unipolar neurons, bipolar neurons, multipolar neurons, Golgi I neurons, Golgi II neurons, anaxonic neurons, pseudounipolar neurons, interneurons, motor neurons, sensory neurons, afferent neurons, efferent neurons, cholinergic neurons, GABAergic neurons, glutamatergic neurons, dopaminergic neurons, serotonergic neurons, histaminergic neurons, Purkinje cells, spiny projection neurons, Renshaw cells, and granule cells, or a combination thereof.

In situ polymerization may be performed in any region or regions of the brain, including in the cerebrum, cerebellum, or brainstem regions of the brain. Brain regions of interest may include, without limitation, the basal ganglia, striatum, medulla, pons, midbrain, medulla oblongata, hypothalamus, thalamus, epithalamus, amygdala, superior colliculus, cerebral cortex, neocortex, allocortex, hippocampus, claustrum, olfactory bulb, frontal lobe, temporal lobe, parietal lobe, occipital lobe, caudate-putamen, external globus pallidus, internal globus pallidus, subthalamic nucleus, substantia nigra, thalamus, and motor cortex regions of the brain.

Interfacing Electronics with Cells and Tissues

The subject methods can be used to allow interfacing of electronics with cells and tissues of living organisms. For example, conductive polymers can be deposited in situ on the plasma membrane or intracellular surfaces of cells, according to the methods described herein, to provide a conductive polymer interface for electrical connections. The conductive polymer interface can be used, for example, with conducting wires for integration into a circuit and to provide connections to electrodes, sensors, and other electronic or electrochemical devices.

In some embodiments, a conductive polymer is deposited in situ on the plasma membrane of a cell to provide a conductive polymer interface to connect the cell with implanted electrodes for electrophysiology measurements. Electrical activity of a single cell, a population of cells, or an organ such as the brain or heart may be measured using electrodes connected to such a conductive polymer interface. In some embodiments, a conductive polymer is deposited in situ on the plasma membrane (e.g., on the extracellular facing side) of an excitable cell, such as a neuron, cardiomyocyte, muscle fiber, or endocrine cell, wherein the conductive polymer is used as an interface to connect electrodes for patch clamp recordings. For example, changes in voltage or electric current associated with one or more ion channels of a cell or neuron action potentials may be measured in this manner.

Electrophysiology measurements can be used to detect brain responses when a subject is exposed to stimuli or performing tasks. Additionally, electrophysiology measurements of brain activity can be taken while the subject is in a resting state (e.g., absence of stimulus or taskless) to allow brain activity to be compared to a subject's “baseline” brain state, i.e., to identify brain regions exhibiting changes in neural activity associated with specific stimuli or tasks.

In some embodiments, the methods described herein are used to evaluate changes in brain function in response to optogenetic perturbation of neural activity. In certain embodiments, optogenetics is used to induce cell-specific perturbations in the brain. For example, optogenetics can be used to excite or inhibit one or more selected neurons of interest using light. For a description of optogenetics techniques, see, e.g., Abe et al., 2012; Desai et al., 2011; Duffy et al., 2015; Gerits et al., 2012; Kahn et al., 2013; Lee et al., 2010; Liu et al., 2015; Ohayon et al., 2013; Weitz et al., 2015; Weitz and Lee, 2013; herein incorporated by reference.

The methods described herein can also be used to evaluate changes in brain function in response to brain stimulation with electrical currents or magnetic fields that are applied to a selected brain area. For example, electrical brain stimulation (EBS) can be used to stimulate a neuron or neural network in the brain through the direct or indirect excitation of its cell membrane by using an electric current. For a description of EBS techniques, see, e.g., Aum et al. (2018) Front Biosci (Landmark Ed) 23:162-182, Tellez-Zenteno et al. (2011) Neurosurg Clin N Am. 22(4):465-75, Padberg et al. (2009) Exp. Neurol. 219:2-13, Nahas et al. (2010) Biol. Psychiatry 67:101-109, Lefaucheur et al. (2010) Exp. Neurol. 223:609-614, Levy et al. (2008) J. Neurosurg. 108:707-714, Hanajima et al. (2002) Clin. Neurophysiol. 113:635-641, Picillo et al. (2015) Brain Stimul. 8:840-842., Canavero (2014) Textbook of Cortical Brain Stimulation. Berlin: De Gruyter Open; herein incorporated by reference. Alternatively, transcranial magnetic stimulation (TMS) can be used to electrically stimulate the brain by electromagnetic induction and can be used to noninvasively stimulate specific regions of the brain. For a description of TMS techniques, see, e.g., Klomjai et al. (2015) Ann Phys Rehabil Med. 58(4):208-213, Lefaucheur (2019) Handb Clin Neurol. 160:559-580, Burke et al. (2019) Handb Clin Neurol. 163:73-92; herein incorporated by reference.

In some embodiments, the methods described herein are used to evaluate changes in brain activity in response to a subject performing cognitive or motor tasks. Such tasks may include, for example, without limitation, tests of memory, intelligence, speech/language, emotion, executive function (e.g., problem solving, planning, organizational skills, selective attention, inhibitory control), visuospatial function, balance, or physical activity/exercise (e.g., walking, running, limb movement). The methods described herein can be used to analyze the intra-regional and inter-regional neural interactions involved in brain activity during particular cognitive or motor tasks and compared to brain activity of the subject in a resting state.

### Nucleic Acids Encoding Polymerization Enzymes

Nucleic acids encoding polymerization enzymes (or fusion proteins comprising a polymerization enzyme linked to a targeting peptide) can be used to genetically modify cells to express a polymerization enzyme at a desired site for in situ polymer synthesis on a membrane or subcellular surface of a cell, as described herein. Nucleic acids encoding a polymerization enzyme can be inserted into an expression vector to create an expression cassette capable of producing a polymerization enzyme in a suitable host cell. The ability of constructs to produce the polymerization enzyme and catalyze polymer formation can be empirically determined (e.g., see Examples 1 and 2 describing detection of the PANI polymer produced by the polymerization enzyme, APEX2, on the extracellular side of the plasma membrane by various microscopy and spectroscopy techniques).

Expression cassettes typically include control elements operably linked to the coding sequence, which allow for the expression of the gene in vivo in the subject species. For example, typical promoters for mammalian cell expression include the SV40 early promoter, a CMV promoter such as the CMV immediate early promoter, the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression.

In some embodiments, a cell-type or tissue-selective promoter is used to localize expression of the polymerization enzyme. For example, to target neurons, a neuron-selective promoter may be used to selectively express the polymerization enzyme in neurons. Exemplary neuron-selective promoters include, without limitation, the synapsin promoter, α-CaMKII promoter fugu promoters, engineered promoters comprising a neuron-restrictive silencer element (NRSE), and h12R-tdTomato and h56D-tdTomato promoters. To target muscle cells, a muscle-selective promoter may be used to selectively express the polymerization enzyme in muscle cells. In some embodiments, the promoter may be selective for skeletal muscle, smooth muscle, or cardiac muscle cells. Exemplary muscle-selective promoters that can be used include, without limitation, the muscle creatine kinase (MCK) promoter, troponin I promoter, and C5-12 promoter.

Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence.

Enhancer elements may also be used herein to increase expression levels of the mammalian constructs. Examples include the SV40 early gene enhancer, as described in Dijkema et al., EMPO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Once complete, the constructs encoding polymerization enzymes can be administered to a subject using standard gene delivery protocols. Methods for gene delivery are known in the art. See, e.g., U.S. Pat. Nos. 5,399,346, 5,580,859, 5,589,466. Genes can be delivered either directly to a vertebrate subject or, alternatively, delivered ex vivo, to cells derived from the subject and the cells reimplanted in the subject.

A number of viral based systems have been developed for gene transfer into mammalian cells. These include adenoviruses, retroviruses (y-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference).

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr Pharm Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2):132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering the polynucleotides of the present invention is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules encoding the polymerization enzymes of interest include those derived from the pox family of viruses, including vaccinia virus and avian poxvirus. By way of example, vaccinia virus recombinants expressing the polymerization enzyme can be constructed as follows. The DNA encoding the particular polymerization enzyme coding sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the coding sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the genes. Recombinant avipox viruses, expressing immunogens from mammalian pathogens, are known to confer protective immunity when administered to non-avian species. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with. respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as, Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the coding sequences of interest (for example, a polymerization enzyme expression cassette) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of genes using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

The expression cassette encoding a polymerization enzyme of interest can also be delivered without a viral vector. For example, the expression cassette can be packaged as DNA or RNA in liposomes prior to delivery to the subject or to cells derived therefrom. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use of liposomes as carriers for delivery of nucleic acids, see, Hug and Sleight, Biochim. Biophys. Acta. (1991.) 1097:1-17; Straubinger et al., in Methods of Enzymology (1983), Vol. 101, pp. 512-527.

Liposomal preparations for use in the present invention include cationic (positively charged), anionic (negatively charged) and neutral preparations, with cationic liposomes particularly preferred. Cationic liposomes have been shown to mediate intracellular delivery of plasmid DNA (Feigner et al., Proc. Natl. Acad. Sci. USA (1987) 84:7413-7416); mRNA (Malone et al., Proc. Natl. Acad. Sci. USA (1989) 86:6077-6081); and purified transcription factors (Debs et al., J. Biol. Chem. (1990) 265:10189-10192), in functional form.

Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. (See, also, Feigner et al., Proc. Natl. Acad. Sci. USA (1987) 84:7413-7416). Other commercially available lipids include (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA (1978) 75:4194-4198; PCT Publication No. WO 90/11092 for a description of the synthesis of DOTAP (1,2-bis(oleoyloxy)-3-(trimethylammonio)propane) liposomes.

Similarly, anionic and neutral liposomes are readily available, such as, from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See, e.g., Straubinger et al., in METHODS OF IMMUNOLOGY (1983), Vol. 101, pp. 512-527; Szoka et al., Proc. Natl. Acad. Sci. USA (1978) 75:4194-4198; Papahadjopoulos et al., Biochim. Biophys. Acta (1975) 394:483; Wilson et al., Cell (1979) 17:77); Deamer and Bangham, Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); Enoch and Strittmatter, Proc. Natl. Acad. Sci. USA (1979) 76:145); Fraley et al., J. Biol. Chem. (1980) 255:10431; Szoka and Papahadjopoulos, Proc. Natl. Acad. Sci. USA (1978) 75:145; and Schaefer-Ridder et al., Science (1982) 215:166.

The DNA and/or peptide(s) can also be delivered in cochleate lipid compositions similar to those described by Papahadjopoulos et al., Biochem. Biophys. Acta (1975) 394:483-491. See, also, U.S. Pat. Nos. 4,663,161 and 4,871,488.

The expression cassette of interest may also be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; McGee J. P., et al., J Microencapsul. 14(2):197-210, 1997; O'Hagan D. T., et al., Vaccine 11(2):149-54, 1993.

Furthermore, other particulate systems and polymers can be used for the in vivo or ex vivo delivery of the nucleic acid of interest. For example, polymers such as polylysine, polyarginine, polyornithine, spermine, spermidine, as well as conjugates of these molecules, are useful for transferring a nucleic acid of interest. Similarly, DEAE dextran-mediated transfection, calcium phosphate precipitation or precipitation using other insoluble inorganic salts, such as strontium phosphate, aluminum silicates including bentonite and kaolin, chromic oxide, magnesium silicate, talc, and the like, will find use with the present methods. See, e.g., Feigner, P. L., Advanced Drug Delivery Reviews (1990) 5:163-187, for a review of delivery systems useful for gene transfer. Peptoids (Zuckerman, R. N., et al., U.S. Pat. No. 5,831,005, issued Nov. 3, 1998, herein incorporated by reference) may also be used for delivery of a construct of the present invention.

Additionally, biolistic delivery systems employing particulate carriers such as gold and tungsten, are especially useful for delivering expression cassettes of the present invention. The particles are coated with the expression cassette(s) to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744. Also, needle-less injection systems can be used (Davis, H. L., et al, Vaccine 12:1503-1509, 1994; Bioject, Inc., Portland, Oreg.).

Direct delivery of expression cassette compositions in vivo will generally be accomplished with or without viral vectors, as described above, by injection using either a conventional syringe, needless devices such as Bioject™ or a gene gun, such as the Accell™ gene delivery system (PowderMed Ltd, Oxford, England).

### Kits

Any of the compositions described herein may be included in a kit. In some embodiments, the kit comprises a vector comprising a promoter operably linked to a polynucleotide encoding a fusion protein comprising a polymerization enzyme (e.g., a peroxidase) connected to a targeting peptide. In addition, the kit may include a polymerization initiator and polymer precursors for carrying out the methods of in situ polymer synthesis described herein.

In certain embodiments, the targeting peptide localizes the polymerization enzyme to the plasma membrane or an organelle membrane. In some embodiments, the targeting peptide localizes the polymerization enzyme to the extracellular-facing surface of the plasma membrane. In some embodiments, the targeting peptide is an a-bungarotoxin (BTX) peptide comprising or consisting of the amino acid sequence of SEQ ID NO: 1. In some embodiments, the fusion protein further comprises a detectable label such as a fluorescent or bioluminescent protein.

In some embodiments, the promoter is a neuron-selective promoter (e.g., human synapsin (hSyn) promoter).

In certain embodiments, the polymerization enzyme included in the kit is a peroxidase, and the polymerization initiator is a peroxide. In some embodiments, the peroxidase is an ascorbate peroxidase. For example, the ascorbate peroxidase may include, without limitation, an Apex2 ascorbate peroxidase such as humanized Apex2. In some embodiments, the peroxide is hydrogen peroxide (H₂O₂) or an organic hydroperoxide.

In certain embodiments, the one or more polymer precursors included in the kit comprise one or more of an aniline monomer, an aniline dimer (N-phenyl-1,4-phenylenediamine), and 4-((5,7-di(thiophen-2-yl)-2,3-dihydrothieno[3,4-b][1,4]dioxin-2-yl)methoxy)butane-1-sulfonate (TETS).

In certain embodiments, the kit further comprises a reagent for doping the polymer such as an acid (e.g., p-toluenesulfonic acid).

Compositions can be in liquid form or can be lyophilized and contained in one or more containers. Suitable containers for the compositions include, for example, bottles, vials, syringes, and test tubes. Containers can be formed from a variety of materials, including glass or plastic. The kit can further comprise a container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

In addition to the above components, the subject kits may further include (in certain embodiments) instructions for practicing the subject methods. In some embodiments, instructions for using the polymerization enzyme for in situ polymer synthesis on a membrane or subcellular surface of a cell are provided in the kits. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Utility

The disclosed methods for in situ assembly of polymers at targeted locations in cells will find many uses in research and development. The subject methods will find use in various applications, including development of sensors, drug delivery, regenerative medicine, and diagnostics. For example, the subject methods can be used to create bioelectronic technology platforms interfaced with cells, tissues, and organs of living organisms. In situ polymer synthesis can be used to provide a conductive polymer interface, which can be used with conducting wires for integration into a circuit within cells and tissue and to provide connections to electrodes, sensors, and other electronic or electrochemical devices. Genetically targeted electroactive, conductive, or insulating polymers may be deposited in situ to alter capacitance, conductance, or insulation properties of any surface or subcellular membrane or compartment in cells. In particular, the subject methods will find many uses in the neuroscience field. For example, in situ polymer synthesis can be used to provide an electronic interface for attachment of electrodes for electrophysiology measurements and cell-type-specific neuromodulation. In situ polymer synthesis can also be used in the field of regenerative medicine to create a durable structure to replace damaged tissue.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-47 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method for in situ polymer synthesis on a membrane or subcellular surface of a cell, the method comprising:
a) expressing a polymerization enzyme on the membrane or subcellular surface; and
b) contacting the cell with one or more polymer precursors and a polymerization initiator, wherein the enzyme catalyzes polymerization of the polymer precursors resulting in production of the polymer on the membrane or the subcellular surface.

2. The method of aspect 1, wherein the membrane is a plasma membrane or an organelle membrane.

3. The method of aspect 2, wherein the polymerization enzyme is localized to an extracellular side of the plasma membrane.

4. The method of any one of aspects 1 to 3, wherein the polymer is an electroactive, conductive, or insulating polymer.

5. The method of aspect 4, wherein the conductive polymer is a polyaniline (PANI) polymer, a poly(3,4-ethylenedioxythiophene) (PEDOT) polymer, a 4-((5,7-di(thiophen-2-yl)-2,3-dihydrothieno[3,4-b][1,4]dioxin-2-yl)methoxy)butane-1-sulfonate (TETS) polymer, or a polyaniline-polyTETS (PANI-PTETS) polymer.

6. The method of aspect 5, wherein the conductive polymer is PANI and the polymer precursors are an aniline monomer and an aniline dimer (N-phenyl-1,4-phenylenediamine).

7. The method of aspect 6, wherein the aniline monomer and the aniline dimer are at a 1:1 molar ratio.

8. The method of any one of aspects 1 to 7, wherein the cell is a neuron, cardiomyocyte, muscle fiber, or endocrine cell.

9. The method of aspect 8, wherein the neuron is an inhibitory motor neuron or an excitatory motor neuron.

10. The method of any one of aspects 1 to 9, wherein the polymerization enzyme is a peroxidase and the polymerization initiator is a peroxide.

11. The method of aspect 10, wherein the peroxidase is an ascorbate peroxidase.

12. The method of aspect 11, wherein the ascorbate peroxidase is Apex2.

13. The method of aspect 11 or 12, wherein the ascorbate peroxidase is humanized.

14. The method of any one of aspects 10 to 13, wherein the peroxide is hydrogen peroxide.

15. The method of any one of aspects 1 to 14, wherein the polymerization enzyme is provided by a vector comprising a promoter operably linked to a polynucleotide encoding a fusion protein comprising the polymerization enzyme connected to a targeting peptide.

16. The method of aspect 15, wherein the targeting peptide localizes the enzyme to the plasma membrane or an organelle membrane.

17. The method of aspect 16, wherein the targeting peptide localizes the enzyme to the extracellular-facing surface of the plasma membrane

18. The method of aspect 17, wherein the targeting peptide comprises or consists of an α-bungarotoxin (BTX) motif comprising or consisting of the amino acid sequence of SEQ ID NO:1.

19. The method of any one of aspects 15 to 18, wherein the fusion protein further comprises a detectable label.

20. The method of aspect 19, wherein the detectable label is a fluorescent or bioluminescent protein.

21. The method of any one of aspects 15 to 20, wherein the promoter is a human synapsin (hSyn) promoter.

22. The method of any one of aspects 15 to 21, wherein the fusion protein further comprises a channelrhodopsin.

23. The method of any one of aspects 15 to 22, wherein the vector is an adeno-associated virus (AAV) vector.

24. The method of any one of aspects 1 to 23, wherein the cell is a live cell or a fixed cell.

25. The method of any one of aspects 1 to 24, wherein the cell is in a tissue, an organoid, or a subject.

26. The method of aspect 25, wherein the tissue is intact living tissue.

27. The method of aspect 25 or 26, wherein the tissue is neural tissue or brain tissue.

28. The method of aspect 25, wherein the subject is an invertebrate or vertebrate.

29. The method of aspect 25 or 28, wherein the subject is a model organism.

30. The method of any one of aspects 1 to 30, further comprising doping the polymer with an acid.

31. The method of aspect 30, wherein the acid is p-toluenesulfonic acid.

32. The method of aspect 31, wherein the polymer is an emeraldine salt of polyaniline (PANI).

33. The method of any one of aspects 2 to 32, wherein the polymer changes the capacitance, conductance, or insulation properties of the plasma membrane or organelle membrane.

34. The method of any one of aspects 1 to 33, further comprising performing optogenetics, electrophysiological measurements, imaging, or spectroscopy, or a combination thereof on the cell.

35. The method of aspect 34, wherein said imaging comprises performing bright-field microscopy, epi-fluorescence microscopy, confocal microscopy, electron microscopy, or a combination thereof.

36. The method of aspect 34, wherein said spectroscopy comprises ultraviolet-visible (UV-vis) spectrophotometry, X-ray photoelectron spectroscopy (XPS), or near edge X-ray absorption fine structure (NEXAFS) spectroscopy, or a combination thereof.

37. The method of any one of aspects 2 to 36, further comprising attaching an electrode to the conductive polymer, wherein the conductive polymer forms a conductive interface between the cell and the electrode.

38. The method of aspect 37, wherein the electrode comprises a biocompatible material.

39. The method of aspect 38, wherein the biocompatible material comprises platinum, titanium, silver, gold, graphite or other conductive carbon material, indium tin oxide (ITO), fluorine-doped tin oxide (FTO), or a metal alloy or an oxide comprising at least one of tin, platinum, titanium, silver, or gold.

40. The method of any one of aspects 37 to 39, wherein the electrode is produced by depositing the biocompatible material on the surface of the polymer.

41. The method of any one of aspects 1 to 40, further comprising exposing the cell to a stimulus.

42. The method of aspect 42, wherein the stimulus is selected from the group consisting of an electrical current, a drug, a ligand for a receptor, a ligand for an ion channel, a ligand for an ion transporter, a hormone, and a second messenger.

43. The method of aspect 41, further comprising genetically modifying the cell to express a light-sensitive ion channel, wherein the stimulus is light that activates the light-sensitive ion channel.

44. The method of aspect 43, wherein the light-sensitive ion channel is selected from the group consisting of a channelrhodopsin, a halorhodopsin, and an archaerhodopsin.

45. The method of any one of aspects 41 to 44, wherein the cell is a neuron.

46. A kit comprising one or more polymer precursors, hydrogen peroxide, and a vector comprising a promoter operably linked to a polynucleotide encoding a fusion protein comprising the peroxidase connected to a targeting peptide, wherein the targeting peptide localizes the peroxidase to the extracellular-facing surface of the plasma membrane.

47. The kit of aspect 46, wherein the one or more polymer precursors comprise one or more of an aniline monomer, an aniline dimer (N-phenyl-1,4-phenylenediamine), and 4-((5,7-di(thiophen-2-yl)-2,3-dihydrothieno[3,4-b][1,4]dioxin-2-yl)methoxy)butane-1-sulfonate (TETS).

48. The kit of aspect 47, further comprising p-toluenesulfonic acid.

49. A method for in situ polymer synthesis in brain tissue, the method comprising:
a) expressing a polymerization enzyme on the plasma membranes of a plurality of neurons in the brain; and
b) contacting the brain tissue with one or more polymer precursors and a polymerization initiator, wherein the enzyme catalyzes polymerization of the polymer precursors resulting in production of the polymer on the plasma membranes of a plurality of neurons in the brain.

50. The method of aspect 49, wherein the plurality of neurons comprises inhibitory motor neurons, excitatory motor neurons, or a combination thereof.

51. The method of aspect 49 or 50, wherein the polymerization enzyme is provided by a vector comprising a promoter operably linked to a polynucleotide encoding a fusion protein comprising the polymerization enzyme connected to a targeting peptide.

52. The method of aspect 51, wherein the targeting peptide localizes the enzyme to the extracellular-facing surface of the plasma membrane

53. The method of aspect 52, wherein the targeting peptide comprises or consists of an a-bungarotoxin (BTX) motif comprising or consisting of the amino acid sequence of SEQ ID NO:1.

54. The method of any one of aspects 49 to 53, wherein the promoter is a human synapsin (hSyn) promoter.

55. The method of any one of aspects 49 to 54, wherein the polymer is a conductive polymer.

56. The method of aspect 55, wherein the conductive polymer is a polyaniline (PANI) polymer, a poly(3,4-ethylenedioxythiophene) (PEDOT) polymer, a 4-((5,7-di(thiophen-2-yl)-2,3-dihydrothieno[3,4-b][1,4]dioxin-2-yl)methoxy)butane-1-sulfonate (TETS) polymer, or a polyaniline-polyTETS (PANI-PTETS) polymer.

57. A method for in situ polymer synthesis on a plasma membrane of a cell, the method comprising:
a) expressing a peroxidase on an extracellular-facing surface of the plasma membrane; and
b) contacting the cell with one or more polymer precursors and a peroxide, wherein the peroxidase catalyzes production of the polymer on the extracellular-facing surface of the plasma membrane.

58. A method for in situ polymer synthesis on a membrane or subcellular surface of a cell and imaging the cell, the method comprising:
a) expressing an enzyme on the membrane or subcellular surface, wherein the enzyme is capable of catalyzing polymerization of the polymer;
b) contacting the cell with one or more polymer precursors and a polymerization initiator, wherein the enzyme catalyzes production of the polymer on the membrane or the subcellular surface; and
c) imaging the cell.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLE 1

### Genetically-Targeted Chemical Assembly

Here we consider if individual cells, and types of cells, within intact biological systems, might be co-opted genetically to build new physical structures with desired form and function. Recent advances in nanoscience have shown that incorporation of miniaturized electrical components onto cellular membranes can change intrinsic cellular activity (8-10), while lacking capability to address genetically-specified cellular components within intact tissue or behaving animals. Other methods currently exist for polymerizing new structures within cells of biological tissues, notably in the hydrogel-tissue chemistry variants (11,12) which leverage the ability of small monomers to diffuse efficiently throughout cells of tissues prior to triggered polymerization (13-17). Although also not genetically targeted, electroactive (e.g. conductive) polymers have been directly synthesized through electrochemical polymerization inside living tissue to reduce electrochemical impedance; free diffusion of monomers into living tissue and reduced local impedance have thus been achieved apparently without disrupting cell function (18). Here, we sought to bring the rapidly-expanding power and potential of cell-type targeting in biological systems to achieve biocompatible in vivo synthesis of conductive polymers within genetically-specified cellular and subcellular compartments in intact living animals.

We began by exploring derivation of conductive polymers from polyaniline (PANI) and poly(3,4-ethylenedioxythiophene) (PEDOT), hypothesizing these could be suitable electronic materials for building genetically-targeted in situ-synthesized electroactive structures. These polymers were chosen for aqueous synthesis important for biological systems, and for their ability for dual conduction of electrons and ions which significantly reduces local electrochemical impedance (19) when interfacing electronics with corresponding cells and tissues of living organisms. However, there is currently no technology that would allow the generation of a genetically-targetable form of conductive polymer synthesis from these or any other raw materials.

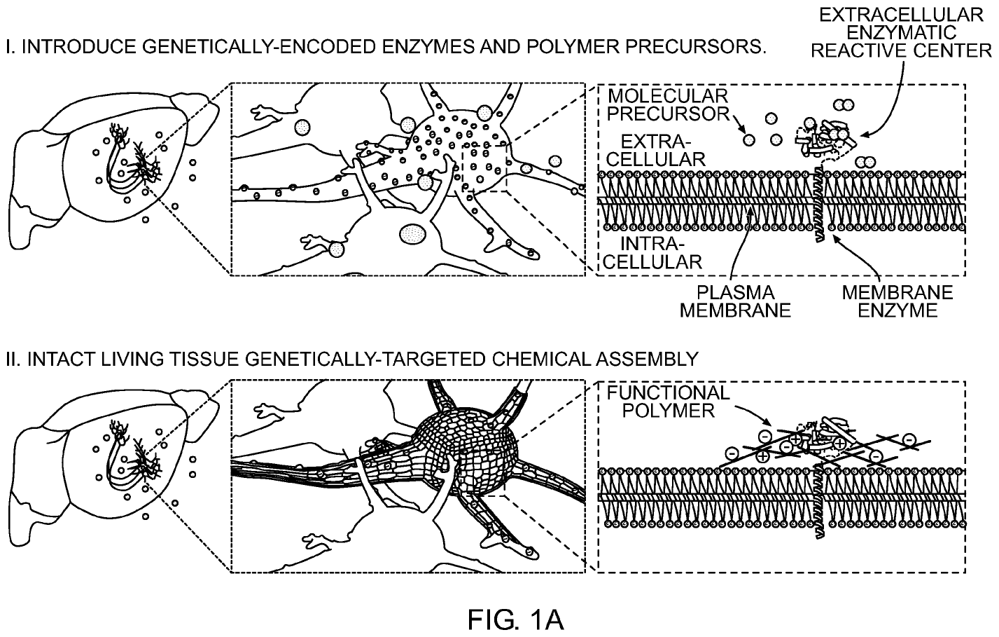

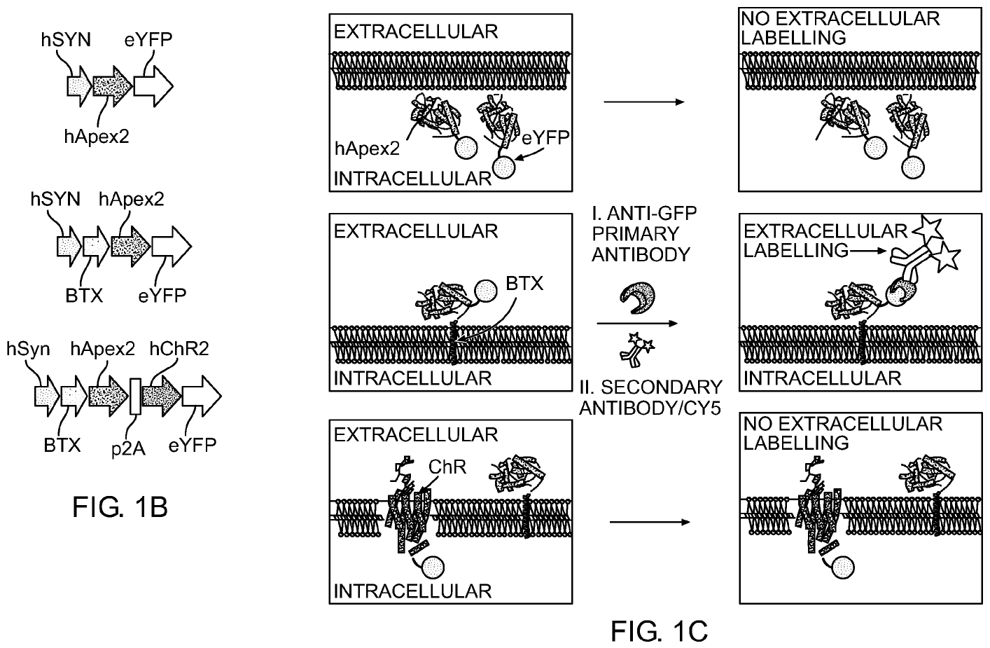

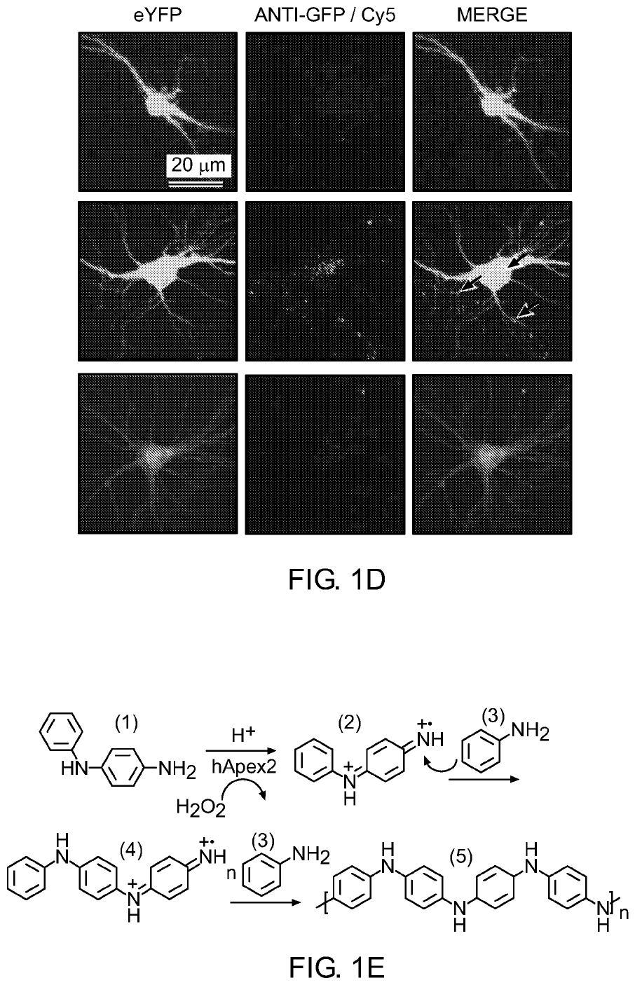

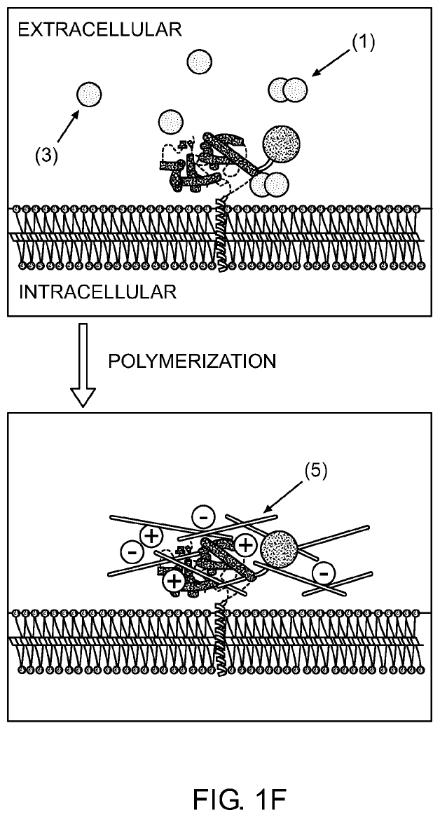

To achieve this fundamentally novel process, we designed a single gene/single enzyme-facilitated polymerization concept, using chemically-modified conductive monomers (FIG. 1A). The crucial design consideration of the monomer is that it cannot polymerize independently in air or by hydrogen peroxide (H₂O₂) unless triggered by the enzyme required for polymerization, which can be expressed in specific types of cells guided by genetic and/or anatomical targeting methods. Perfusion of small-molecule conductive polymer precursors capable of rapid diffusion through intact tissue (Step I) is followed by a radical initiated polymerization step at the reactive center of the genetically-targeted enzyme. By design, due to both the short mean diffusion path length of radicals in aqueous solution, and the low solubility of conductive polymers, newly synthesized conductive polymers will be deposited onto targeted cells and subcellular locations (in this case, onto the plasma membrane—a design feature for limiting adverse effects of synthesis on native intracellular signaling and chemistry (Step II).

There have been reports on the use of peroxidases (20-21), which enzymatically catalyze the synthesis of conductive polymers in the presence of H₂O₂. However, such peroxidases can only polymerize conductive polymers in vitro and under extremely harsh conditions: high concentrations of hydrogen peroxide (>1 mM), low pH (pH=1), and high monomer concentrations (>10 mM); such conditions are far from applicable to living systems. These harsh reaction conditions generally preclude in vivo use, particularly since the reactive reagents (monomer and H₂O₂) would not be readily accessible to reactive centers of peroxidases expressed inside cells, due to low permeability of the plasma membrane lipid bilayer. Therefore, an initial goal was to move towards biocompatible synthesis by addressing two important aspects: (i) ensuring access to the reactive center of the enzyme, and (ii) enabling polymerization in pH-neutral/biocompatible conditions.

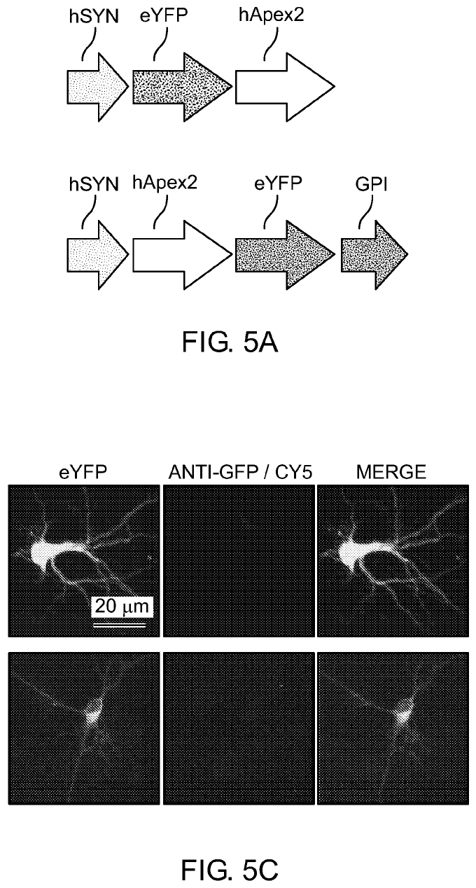

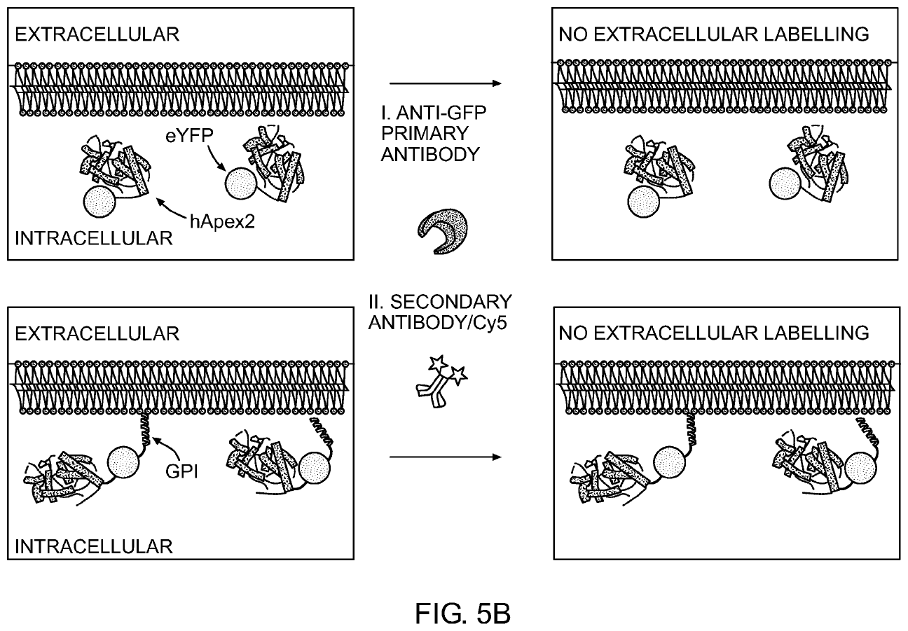

To address the first aspect (FIGS. 1B-1D, FIG. 5), we began by protein engineering to tether the initiator enzyme to the plasma membrane, in a configuration placing reactive centers on the extracellular side of living cells. This is in contrast to previously reported in vitro methods focusing on the intracellular localization of overexpressed enzymes in fixed tissues (22). For the enzyme, we selected an engineered and humanized version of ascorbate peroxidase hApex2, a putative polymerization catalyst given its reported utility in histology (22). We constructed a panel of adeno-associated virus (AAV) vector variants containing hApex2 driven by the synapsin (hSyn) promoter, in some cases fused with the 13-mer BTX peptide (for extracellular membrane-delimited expression; 23), enhanced yellow fluorescent protein (eYFP, for tracking localization), and/or accompanied by a channelrhodopsin (for simultaneous optical-control capability) (FIG. 1B). We then infected cultured rat hippocampal neurons with these viruses (FIGS. 1C-1D) to evaluate expression.

To test extracellular localization of hApex2-eYFP, we carried out intact-cell staining with an anti-green fluorescent protein (GFP) primary antibody prior to cell fixation and without membrane permeabilization, followed by a Cy-5 tagged secondary antibody (FIG. 1C). Of the vector variants, only the BTX-fusion construct showed substantial Cy5 fluorescence signals under these conditions (FIGS. 1C-1D), indicating expression of hApex2-eYFP on the extracellular surface per design. The BTX-hApex2 construct was therefore carried forward into the next stage of experiments, to enable extracellular expression of the enzymatic reaction center; we denote this construct as hApex2(+) and the eYFP-only control as hApex2(−).

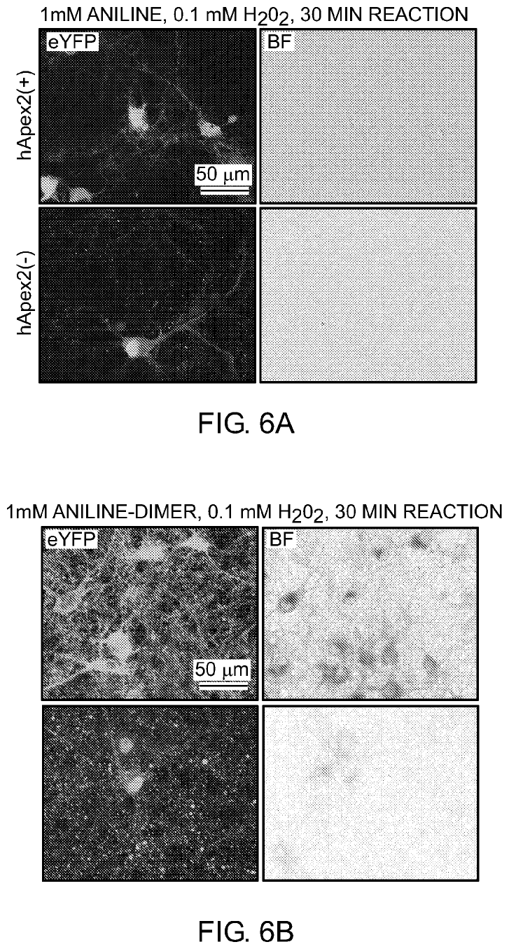

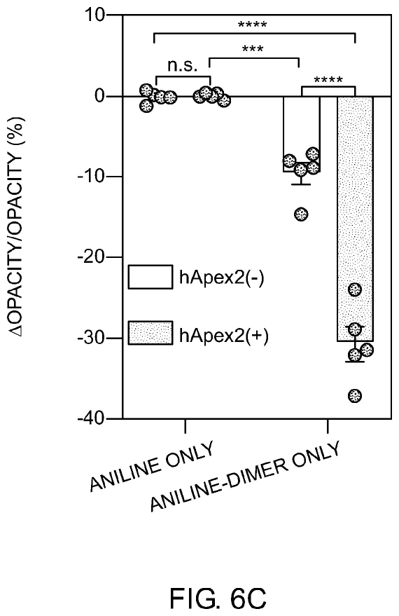

To address the second aspect, we needed to increase reactivity of typical PANI precursors in biocompatible conditions. We sought to minimize the required oxidation potential for the precursor monomers. PANI had been selected for initial testing due to its relatively low oxidation potential (24), but in phosphate-buffered saline (PBS) buffer solution at pH=7.4, hApex2 was unable to effectively polymerize aniline monomers (FIG. 6A). It had previously been reported that using N-phenylenediamine (an aniline dimer) could reduce the oxidation potential required for polymerization of PANI (25). Therefore, we re-designed the reaction composition to include both aniline dimer and monomer (1:1 molar ratio) to enhance the reactivity of PANI precursors (FIG. 1E). In this reaction, the dimer and monomer precursors (0.5 mM) were added to aqueous solution containing H₂O₂ (0.1 mM) and applied to fixed cultured neurons, to test if rapid local polymerization could occur in the presence of extracellular hApex2 (FIG. 1F). Optical characterization (BF channel) of fixed neurons after 15-min reaction confirmed that hApex2(+) neurons appeared to be covered by a dark shell-like structure, while no such deposition occurred on hApex2(−) neurons (FIG. 1G). The mixed precursor solution containing both aniline monomer and dimer seems to be crucial to enable the required specificity as we found that if only aniline-dimer was used (without adding aniline monomer), a non-hApex2-specific reaction product on hApex2(−) neurons was observed (FIGS. 6B, 6C).

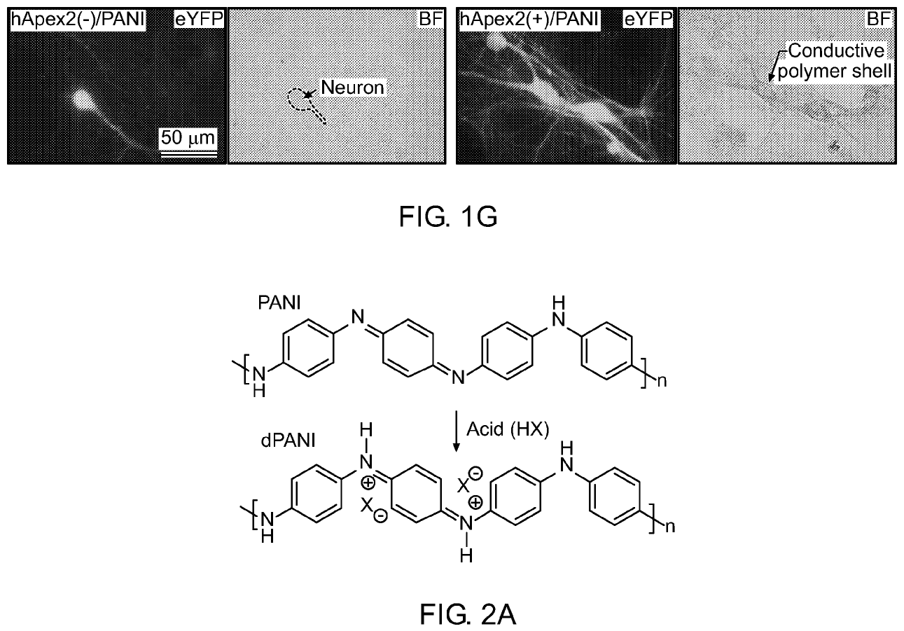

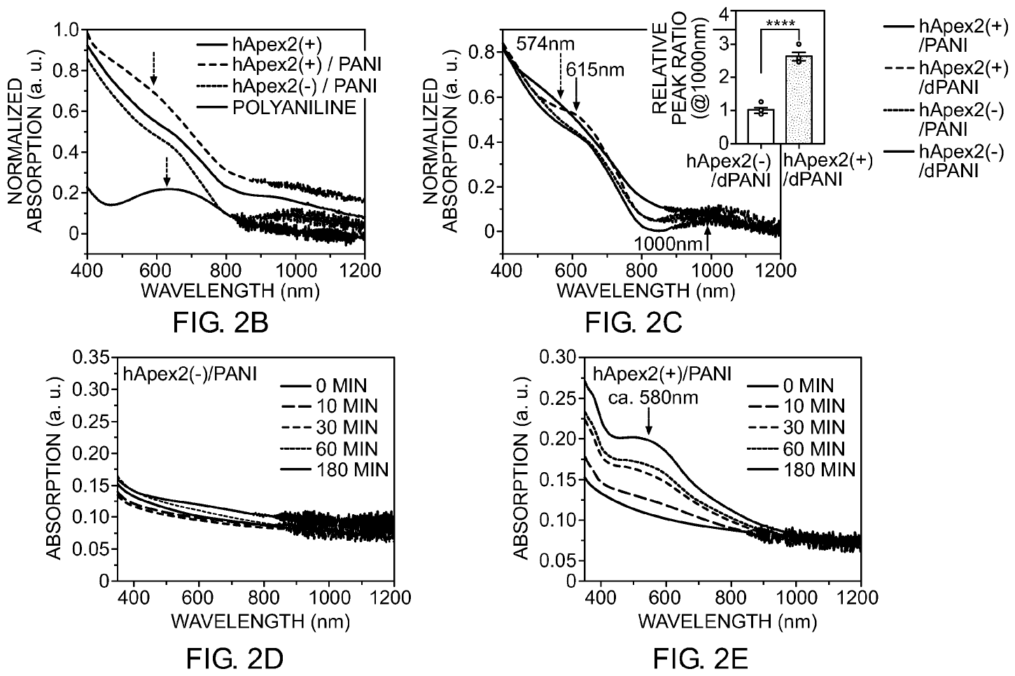

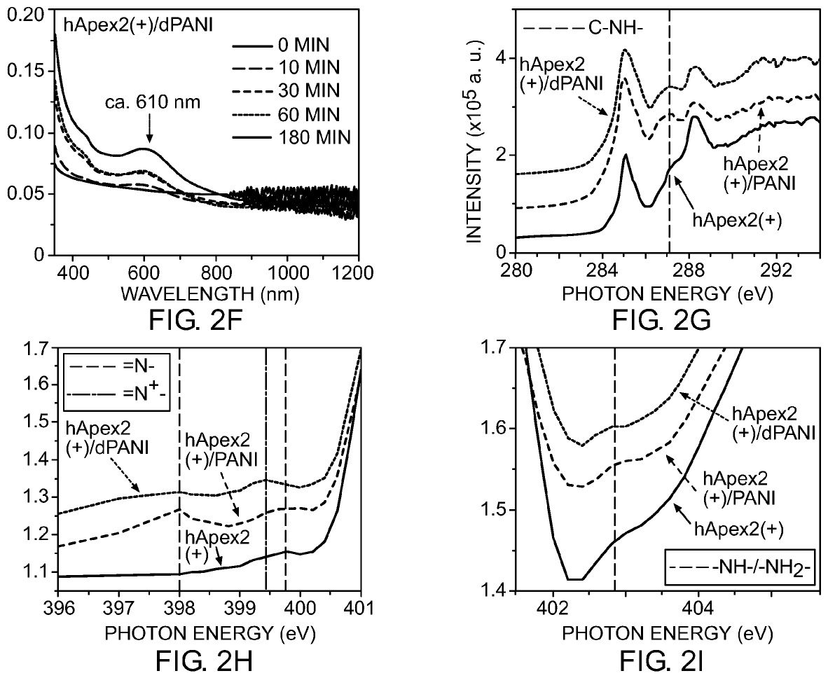

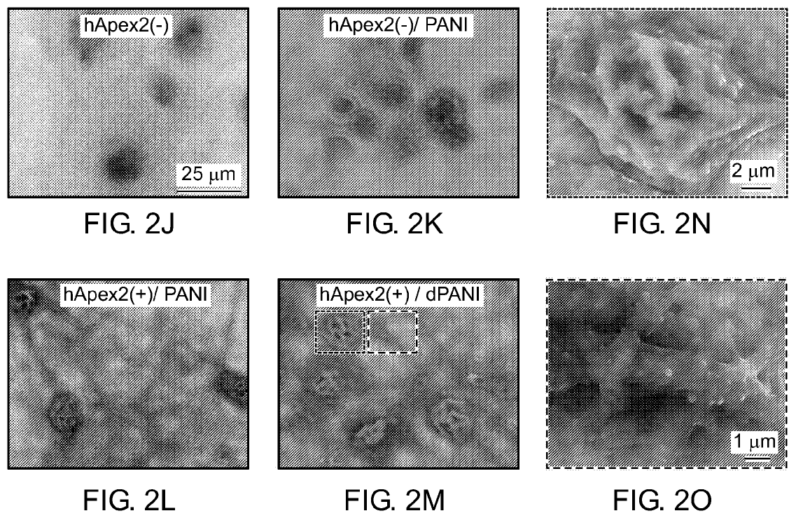

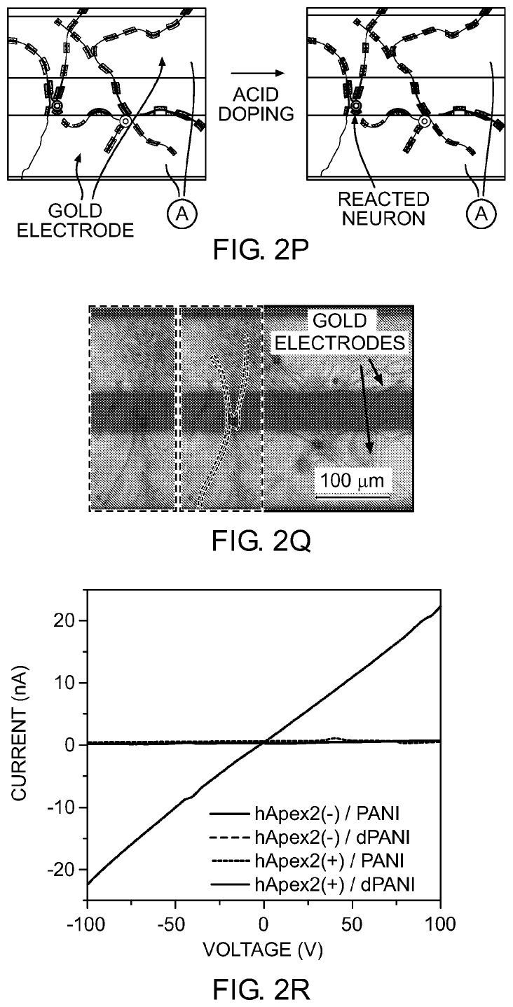

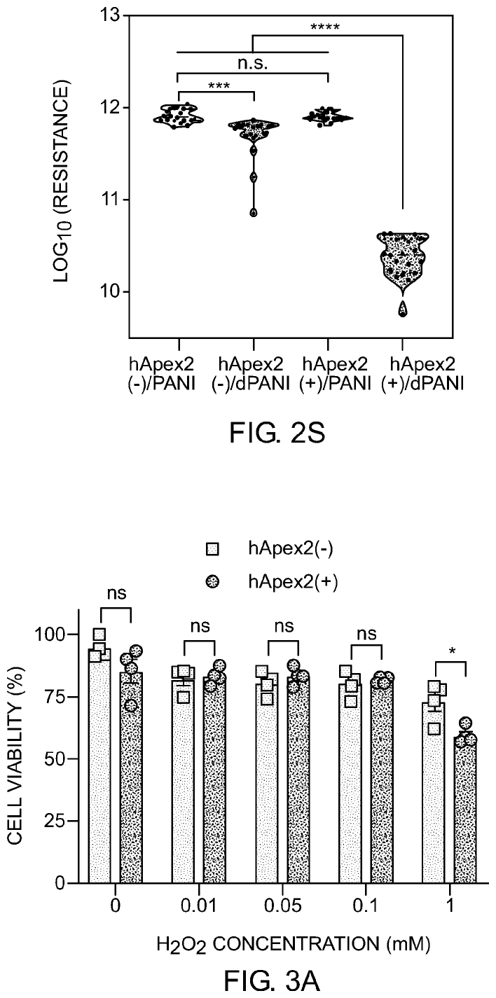

To test if the hApex2-dependent shell-like layers deposited on neurons indeed consisted of conductive PANI, we first used UV-vis absorption spectroscopy to analyze the composition of the surface layer deposited (FIG. 2A). FIG. 2B shows a comparison of normalized absorption spectra among commercially sourced PANI (MW 50 kDa), non-reacted neurons, hApex2(+)/PANI neurons, and hApex2(−)/PANI neurons. The maximum absorption peaks for hApex2(+)/PANI was at ˜574 nm vs. ˜620 nm for commercial high molecular weight PANI, indicating that the PANI synthesized on hApex2(+) neurons are potentially lower molecular weight oligomers.

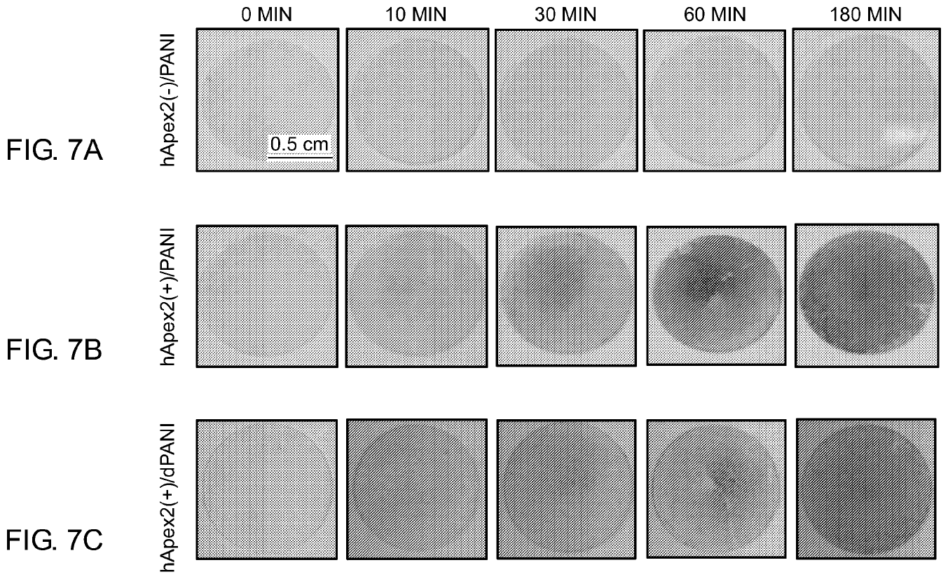

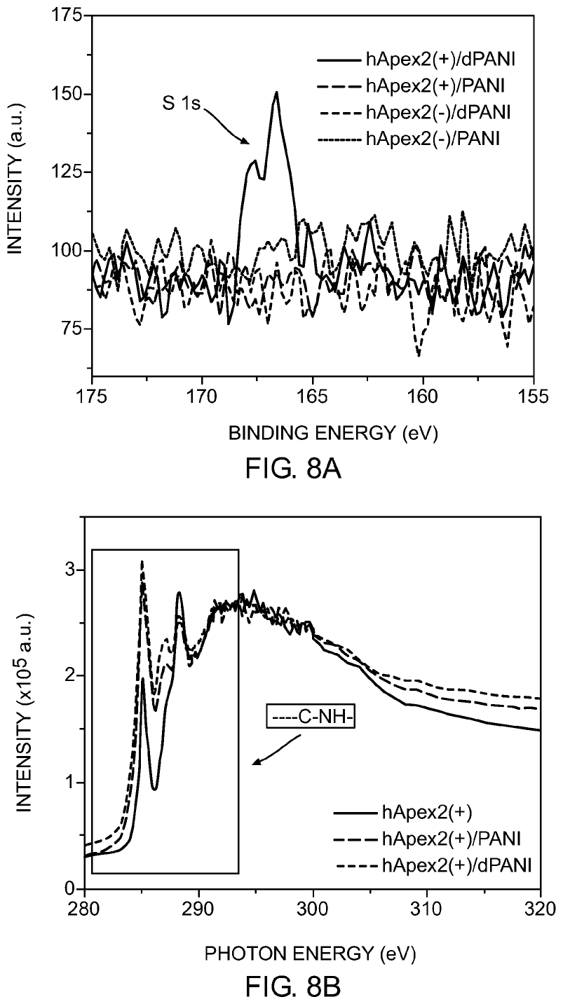

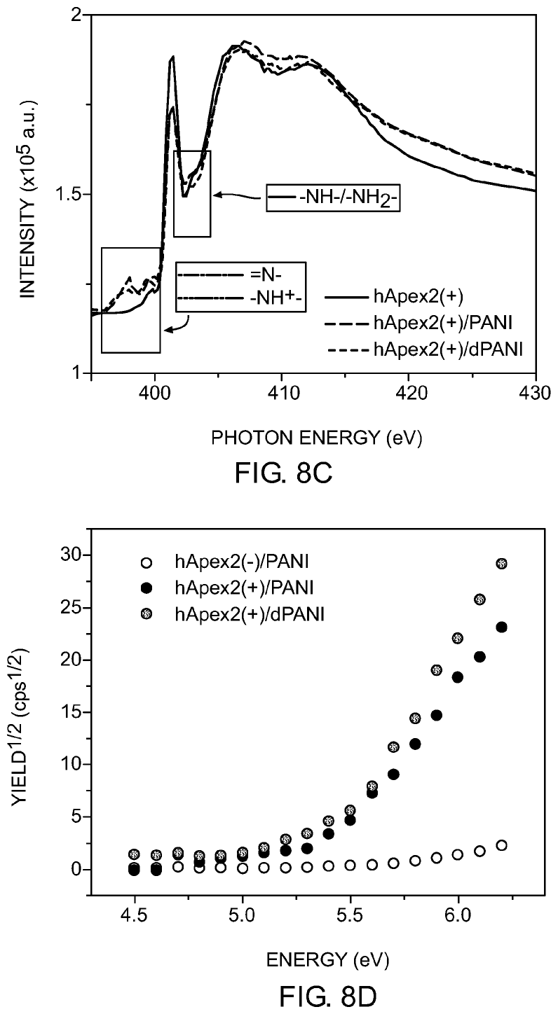

We then doped these fixed-neuron samples by treating with an aqueous solution of 100 mM p-toluenesulfonic acid for 15 minutes and rinsing 3 times in deionized (DI) water. It has been reported that PANI, when doped with acid (FIG. 2A), becomes more conductive (in emeraldine salt form) due to additionally introduced holes, exhibited by notable red shifts in its UV-vis absorption spectra as expected for doped states (26). We observed negligible color changes in PANI monomer-treated hApex2(−) neurons during the reaction time (labeled as hApex2(−)/PANI, FIG. 7A), while in contrast we observed a strong purple color emerging only after a 10-min reaction for the fixed hApex2(+) neurons (labeled as hApex2(+)/PANI, FIG. 7B), which continued to increase for the next 3 hours. A red-shift in absorption was observed for the acidic-doped-PANI (labeled as hApex2(+)/dPANI), along with a change in color from purple to blue-green (FIG. 7C). X-ray photoelectron spectroscopy (XPS) showed an enhanced S-element signal only in the hApex2(+)/dPANI sample, confirming incorporation of p-toluenesulfonic acid (FIG. 8A). FIG. 2C shows the UV-vis spectra comparisons between hApex2(+)/PANI and hApex2(−)/PANI neurons before and after this treatment. The redshift of the peak from ˜574 nm to ˜615 nm, and the emergence of a broader peak at ˜1100 nm for the doped PANI, were only present on hApex2(+)/PANI neurons, which indicates transition to the emeraldine salt form of PANI (26). Importantly, the UV-vis spectra on samples with different reaction time showed that peak wavelength of absorption were maintained over time, suggesting that the increase in reaction time corresponded to the increased amount of PANIs deposited on neurons (FIGS. 2D-2F).

Next, we used near edge X-ray absorption fine structure (NEXAFS) spectrum to confirm the chemical composition of the black particles in the dark shell-like structures (FIGS. 2G-2I, FIGS. 8B-8D). Different types of C—N or C═N features from amines and imines can be identified with NEXAFS (27). FIG. 2G shows the carbon-edge C 1s spectra. The peak at ˜287.1 eV (black dashed line) observed in both hApex2(+)/dPANI and hApex2(+)/PANI samples (red and blue curves, respectively) corresponds to the carbon 1s→π* absorption (originating from carbon atoms with nitrogen as nearest-neighbor) present in aniline moieties, similar as in previous reports (28). These peaks were not present in the hApex2(+) control sample (black curve, fixed hApex2(+) neurons without exposure to the PANI reagent), where only a slight shoulder was observed (as expected from the proteins present in neurons). FIG. 2H shows the nitrogen edge N 1s spectra of the same samples, indicating the changes in oxidation states of the imine and amine nitrogen atoms upon doping and transition of the PANI emeraldine base to PANI emeraldine salt (29). Prior to doping, the spectrum of the hApex2(+)/PANI sample (blue curve) showed distinguishable peaks at ˜397.9 eV and ˜399.7 eV, indicating unprotonated imine nitrogen atoms (═N—, black dashed lines). After doping with p-toluenesulfonic acid (labeled as dPANI), the hApex2(+)/dPANI sample (red curve) revealed that these peaks were diminished and replaced by a single peak at ˜399.5 eV, indicating proton N atoms (═NH⁺—, green dashed line). FIG. 2I shows both UV-vis and NEXAFS characterizations were consistent with previously reported results for commercially-sourced PANI (28), confirming that aromatic, p-conjugated structures from PANI were present within the sample.

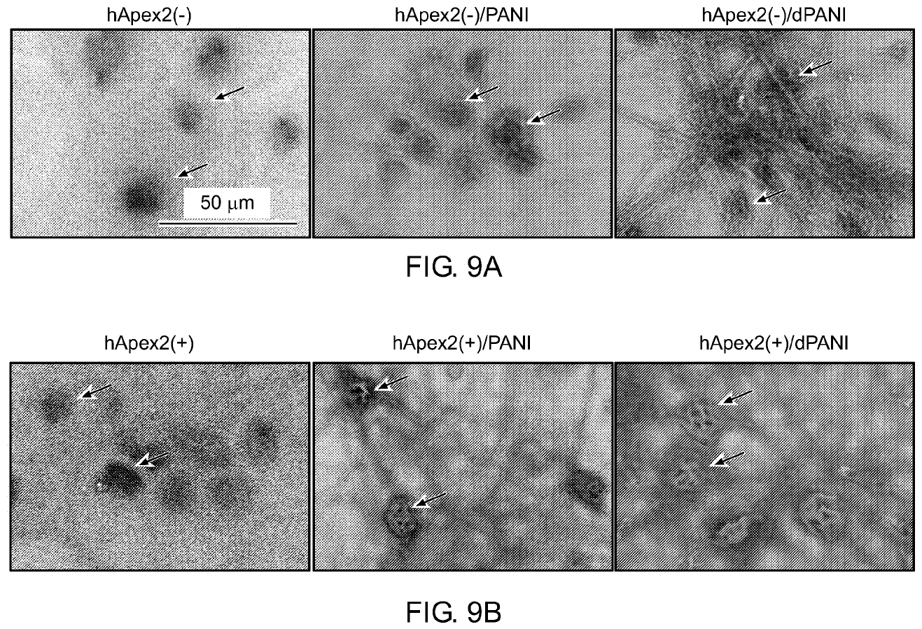

To verify the conductive nature of the PANI layer, we used variable pressure scanning electron microscopy (VP-SEM) to image neurons pre- and post-reaction. Importantly, neurons were not treated with additional high-atomic-number electron-dense stains, such as osmium, and all images were taken with the same EM parameter settings (accelerating voltage, probe current, working distance, beam intensity and exposure time). The images (FIGS. 2J-2L, FIG. 9) of hApex2(+)/PANI neurons showed higher contrast consistent with a shell-like conductive outer layer, with contrast further enhanced by acidic doping (FIGS. 2K-2M). In contrast, hApex2(−) neurons could not be clearly imaged with or without polymerization (FIGS. 2J-2K), consistent with an insulating surface. Notably, high-resolution structural images of neurons, including both soma and neurites, could be directly observed for the Apex2(+)/dPANI samples, suggesting substantial surface conductivity enhancement from the doped PANI coating (FIGS. 2N-2O). The shell-like structures observed by VPSEM were also consistent with the earlier optical imaging showing PANI on the surface of neurons. Since VP-SEM imaging reflects the backscatter intensity of electrons (30), which is generally signal limited and affected by specimen conductivity, we conclude that the improvement in image resolution and signal-to-noise-ratio for hApex2(+) neurons was due to the deposition of a conductive PANI layer.

We further investigated the electrically conductive nature of the coated polymer layer by depositing gold electrodes on the air-dried, fixed neurons (FIGS. 2P, 2O) to measure current-voltage (I-V) curves. Under these conditions, electrical conductivity is expected to only arise from conductive polymers coating the neuronal surfaces. To prevent delamination between gold electrodes to polymers in solution doping process, here hydrochloric acid vapor was used to dope the polymer. FIG. 2R shows representative I-V curves of hApex2(−)/PANI, hApex2(−)/dPANI, hApex2(+)/PANI, and hApex2(+)/dPANI neurons, with hApex2(+)/dPANI showing the lowest resistance as expected. Summary data (FIG. 2S) revealed that: (i) hApex2(−)/dPANI samples exhibited only a slight decrease in resistance upon acid vapor doping, possibly attributable to residual acid molecules, and (ii) hApex2(+)/dPANI samples exhibited ˜2 orders-of-magnitude decrease in resistance.

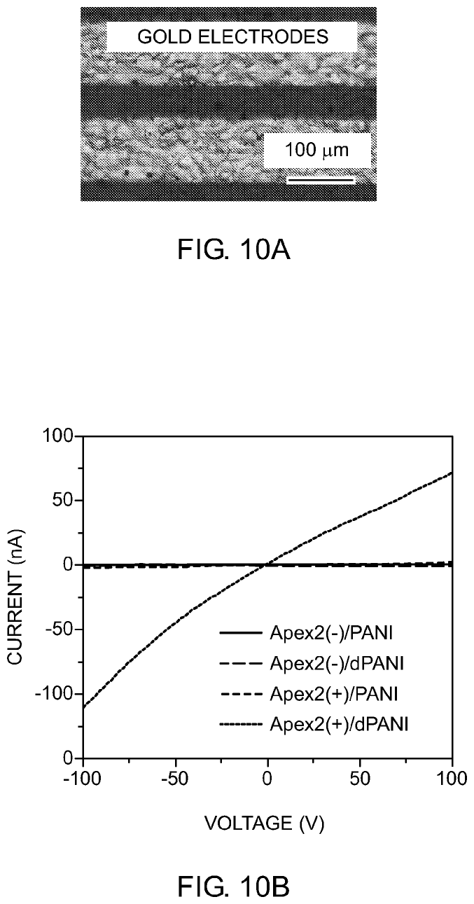

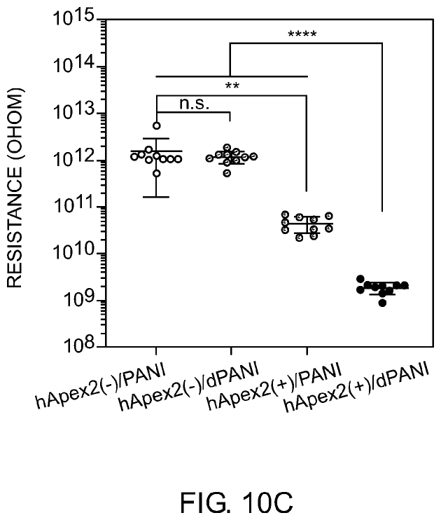

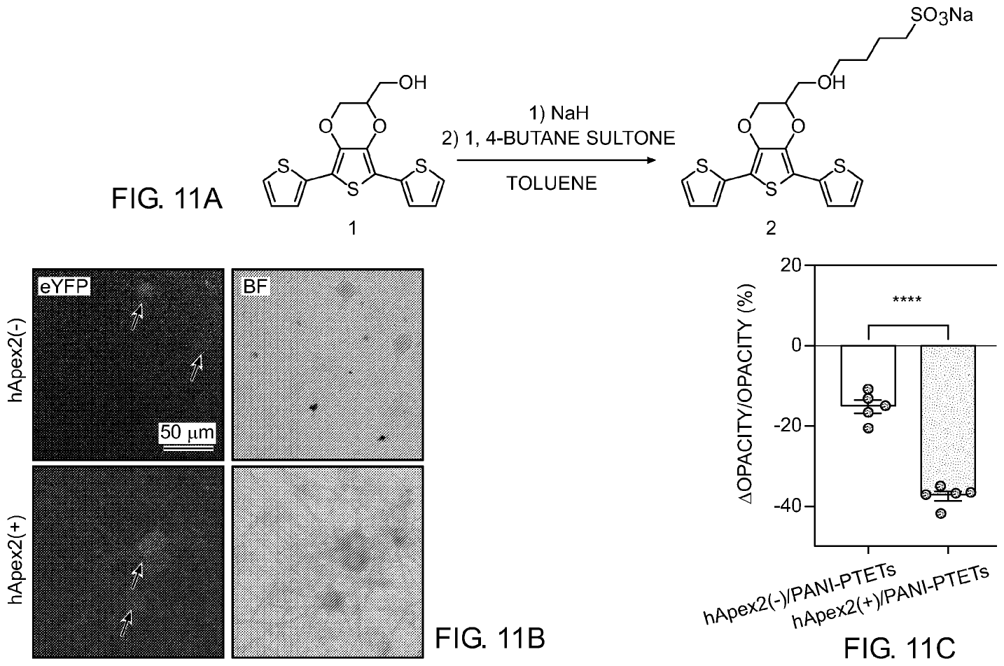

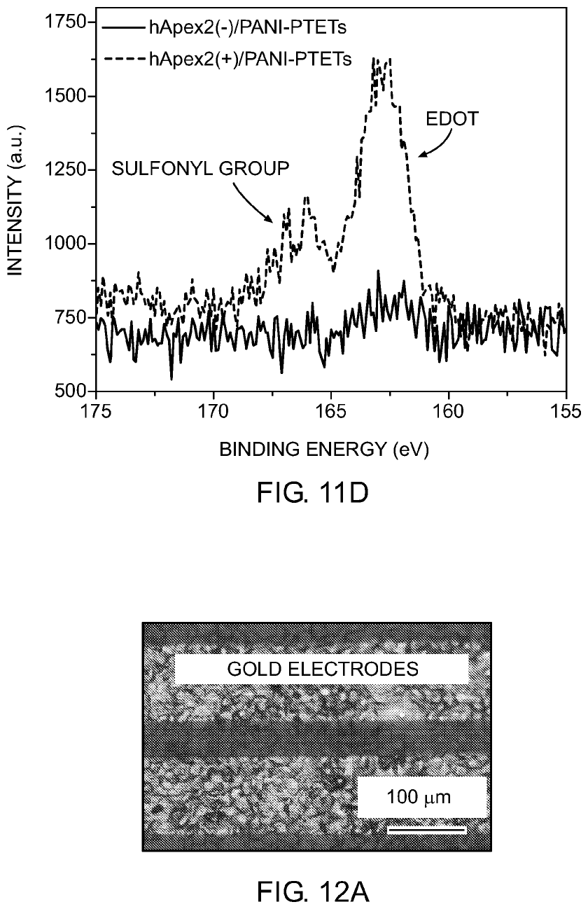

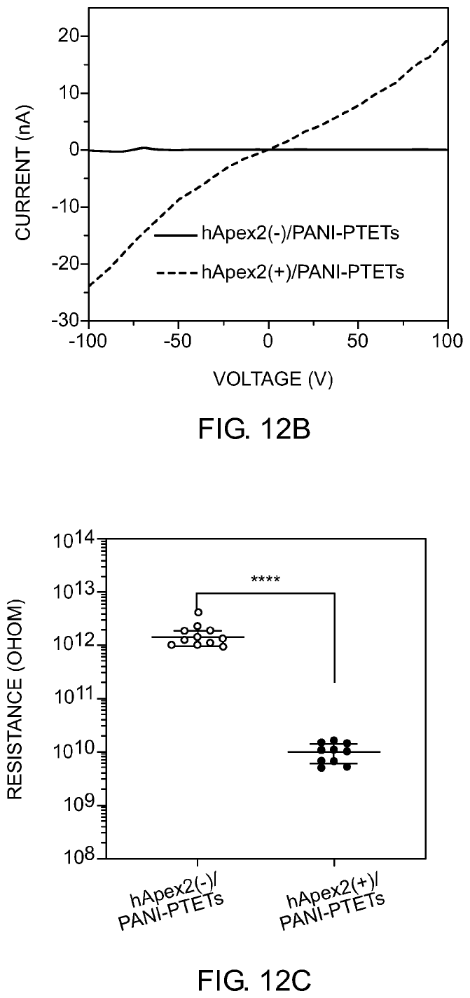

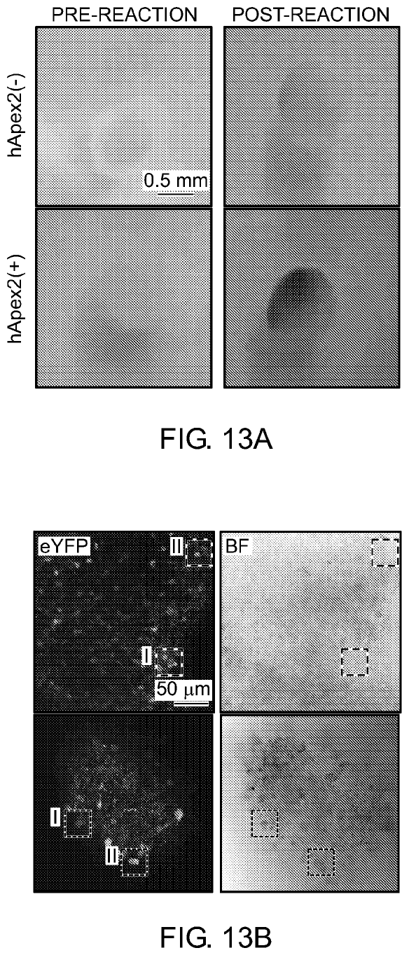

Neurons usually distribute sparsely in 2D culture, making it harder to obtain a percolating conducting network. For additional verifications, we next cultured human embryonic kidney (HEK293T) cells in a confluent 2D cell sheet well-suited for conductivity measurements, (FIG. 10A). HEK cells expressing hApex2-BTX indeed also exhibited reduced resistance after polymerization and doping (FIGS. 10B, 10C), again confirming the conductive nature of the deposited polymer after doping. We used this confluent cell preparation to test monomers for other conductive polymers, including a poly(3,4-ethylenedioxythiophene) (PEDOT)-derivative sodium 4-((5,7-di(thiophen-2-yl)-2,3-dihydrothieno[3,4-b][1,4]dioxin-2-yl)methoxy)butane-1-sulfonate (31, termed TETs). This thiophene-EDOT-thiophene trimer contains a side-chain enabling self-doping (FIG. 11A), which can be polymerized together with the aniline dimer (FIGS. 11B-11D) to give a higher conductivity than the undoped PANI. After polymerization on the confluent HEK cell-sheet and contacted by electrodes (FIG. 12A), we observed a 2 order-of-magnitude reduction in resistance compared to hApex2(−) controls, without the need for further acidic doping (FIGS. 12B-12C). Together, these results demonstrate that hApex2 specifically drives conducting polymer synthesis on cells.

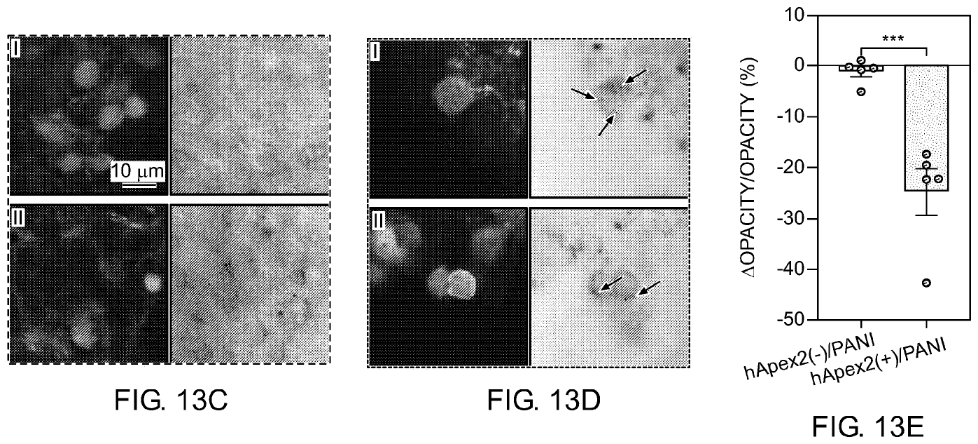

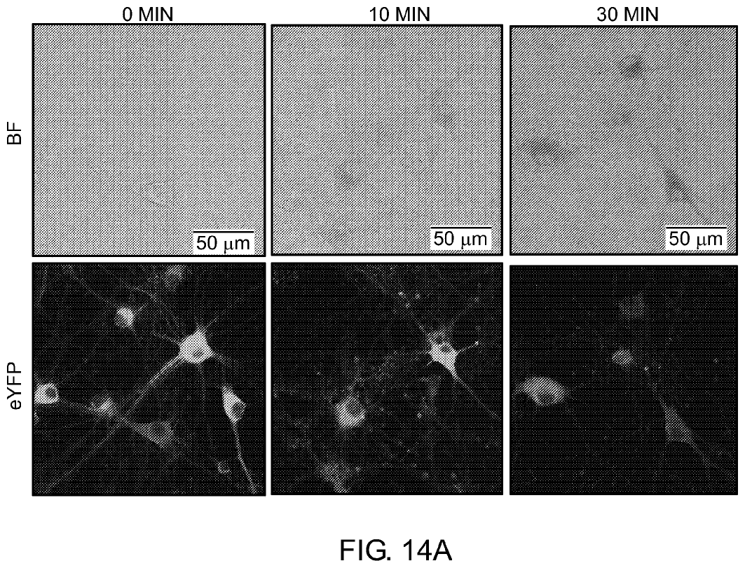

After characterizing functional properties of the polymer in cultured 2D monolayers of cells, we next investigated polymerization in a human, stem-cell derived, 3D region-specific brain organoid system known as human cortical spheroids, or hCS (32, 33), in which BTX-hApex2 expression in neurons is driven by the human synapsin (hSyn) promoter. Cell density in hCS facilitated the diffusion of the conductive polymer precursor and enabled the reaction throughout the tissue in a manner that was easily imaged by bright-field microscopy. Upon exposure to the aniline dimer and monomer reagent, coloration of hApex2(+) hCS changed to purple, while that of hApex2(−) hCS did not (FIG. 13A) indicating no formation of PANI. Clearly detectable particles could be visualized within hApex2(+)/PANI hCS corresponding to locations observed in the eYFP signal (FIG. 13B), and zoomed-in confocal images revealed that the particles could be observed in neurites (FIG. 13C) forming shell-like structures (FIGS. 13D, 13E) just in 2D cultured neurons. Particles could in fact be visualized as deep as 500 μm inside the hCS.

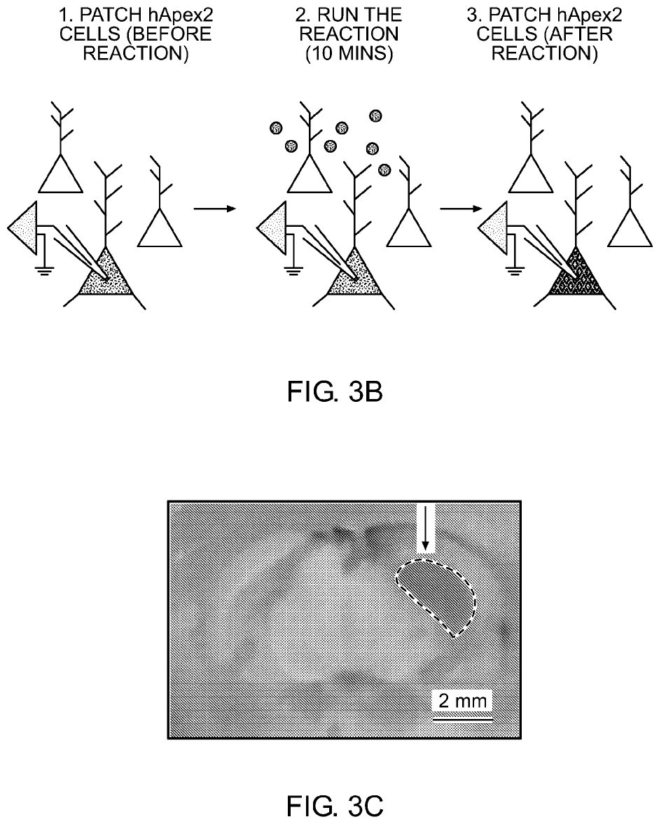

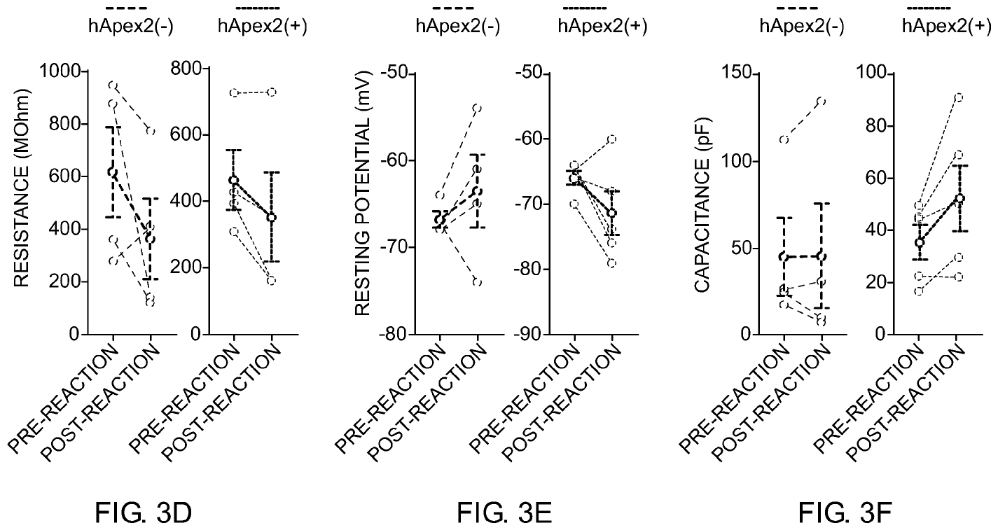

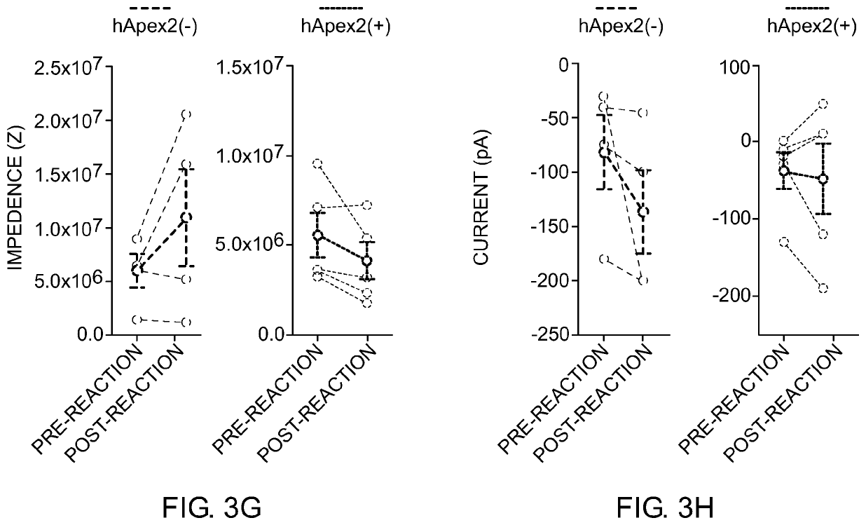

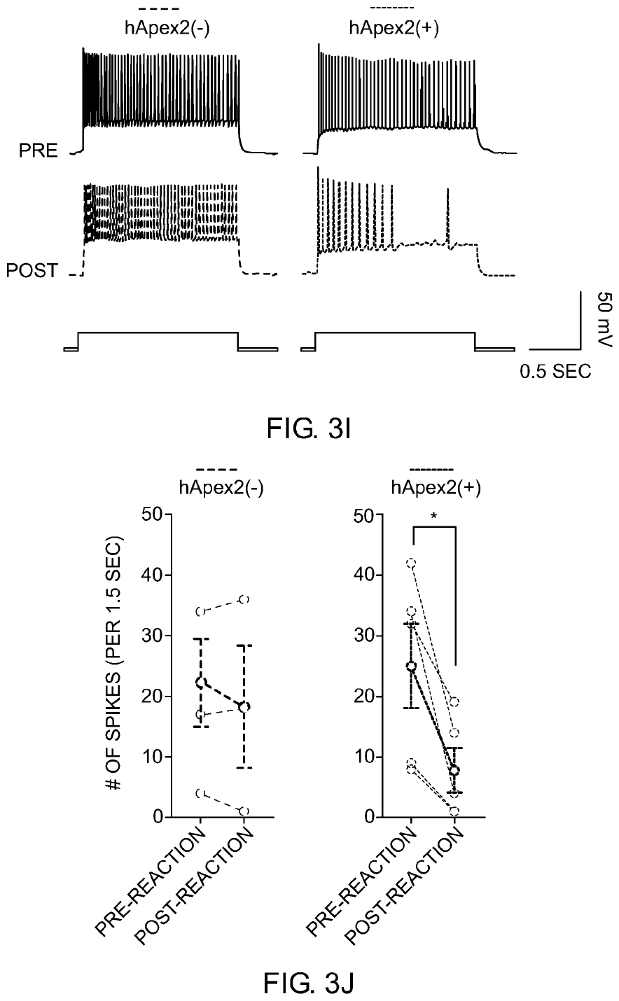

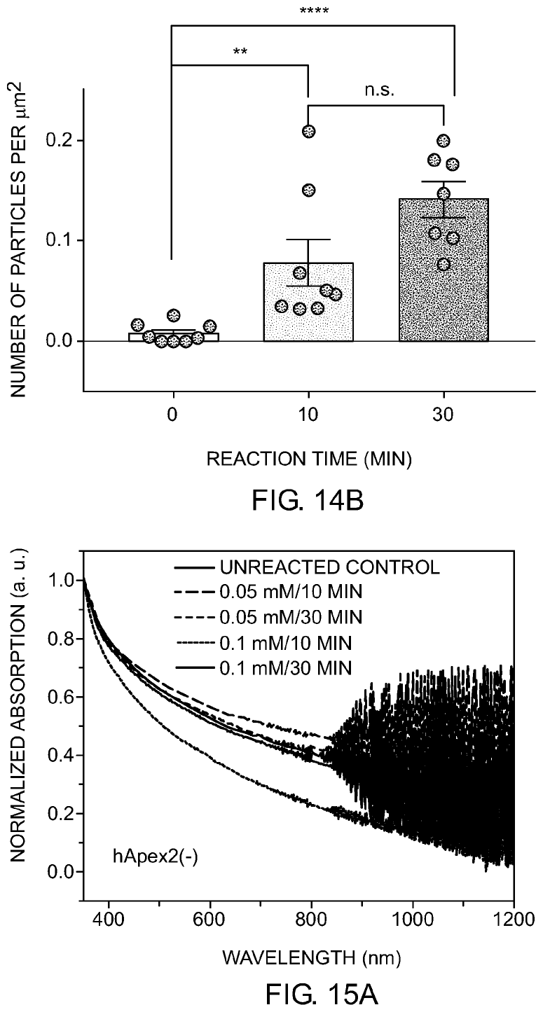

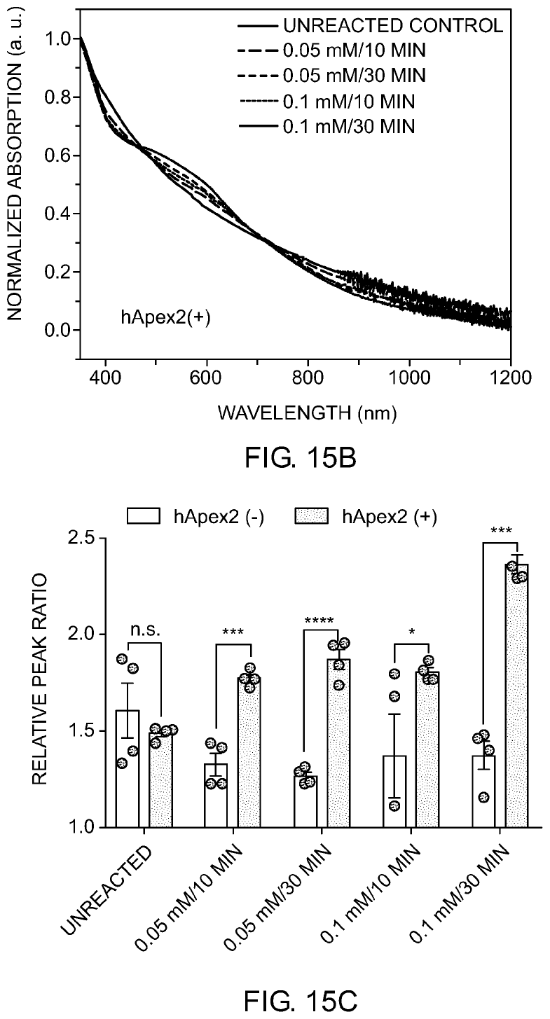

We next proceeded to test polymerization in living systems, to investigate impact upon cellular electrophysiological properties and animal behavior. We first tested the impact of reaction at a fixed monomer/dimer concentration with varying concentrations of H₂O₂, to determine viability by live-dead cell assay. We observed that exposure to the polymer precursor in 0.05 mM H₂O₂ permitted viability (FIG. 3A), even over 10 min of continuous exposure. We then visualized polymerization in living neurons by confocal microscopy (FIG. 14A), which revealed that this reaction condition was sufficient to polymerize shells upon neurons under these biocompatible conditions. Particle density (0.078±0.023 per μm²) was significantly higher than for non-reacted samples (0.008±0.003 per μm²), and at 10-min was comparable to that of 30-min reaction samples (0.142±0.017 per μm²) (FIG. 14B). In addition, UV-vis absorption spectra showed that the distinctive absorption peak at 574 nm could be detected after 10-min reaction in hApex2(+) neurons, but not in hApex2(−) neurons after the same reaction time (FIG. 15).

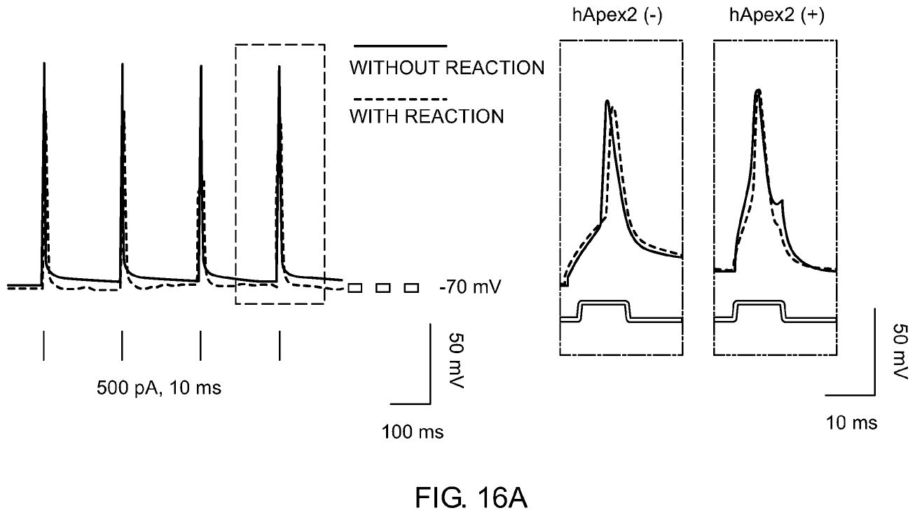

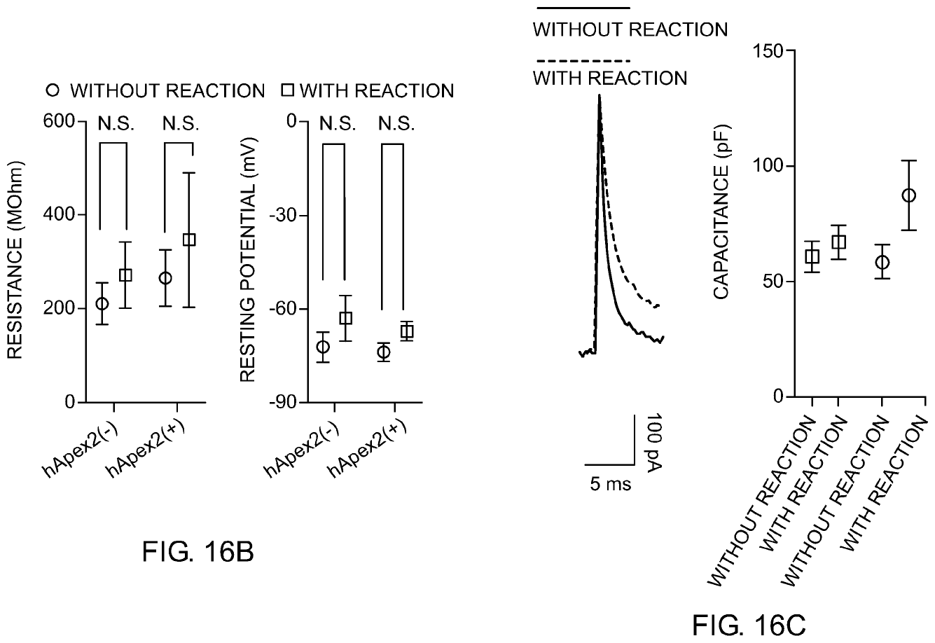

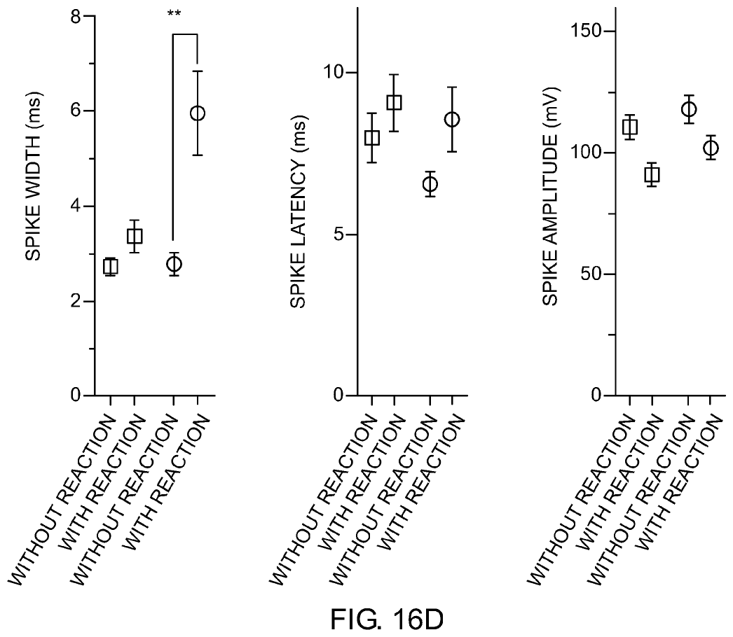

One goal of the genetically-targeted chemical assembly approach is to enable genetically-targeted incorporation of electroactive materials into living tissue. To explore the potential of this approach, we first performed whole-cell patch clamp experiments in hApex2(+) and hApex2(−) cultured hippocampal neurons, before and after polymerization conditions. Current injections (500 pA, 10 ms, 5 Hz) in hApex2(+) neurons elicited robust action potential firing in both pre- and post-polymerization conditions (FIG. 16A). Regarding active membrane properties, while spike width increased after reaction (2.78±0.78 ms vs 5.96±0.88 ms), no significant difference in spike latency or amplitude was observed (FIG. 16D). Regarding passive membrane properties, there was no significant difference in input resistance or resting potential (FIG. 16B), but we observed a ˜30 pF increase in cell capacitance calculated using a 10 mV hyperpolarizing step (58.68±7.31 pF vs 87.35±15.02 pF, FIG. 16C). This effect is consistent with previous reports wherein nanoparticle introduction was found to increase cell capacitance (34-35), and consistent with the nature of the intervention in this case as well, in which conductive charged nanoparticles are deposited onto the phospholipid bilayer.

To further investigate properties and performance of living mammalian neural circuitry after targeted generation of conductive polymers, after in vivo hApex2 expression we conducted whole-cell patch-clamp characterization in acute living brain slices before and after polymerization (FIG. 3B). Four weeks after injection of AAVdj-BTX-hApex2-eYFP vectors into hippocampus and motor cortex, robust expression of hApex2 was observed. Consistent with our previous results, targeted synthesis of PANI in hApex2(+) neurons was observed after only a 10-min of polymerization (FIG. 3C), and whole-cell patch clamp of the same neurons before and after polymerization showed increased membrane capacitance (FIG. 3F) (consistent with in vitro expression) and no effect of hApex2-dependent polymerization upon most passive membrane properties, including membrane resistance and resting potential (FIG. 3D-E), membrane electrochemical impedance at 1000 Hz (FIG. 3G) and holding current at resting potential (FIG. 3H).

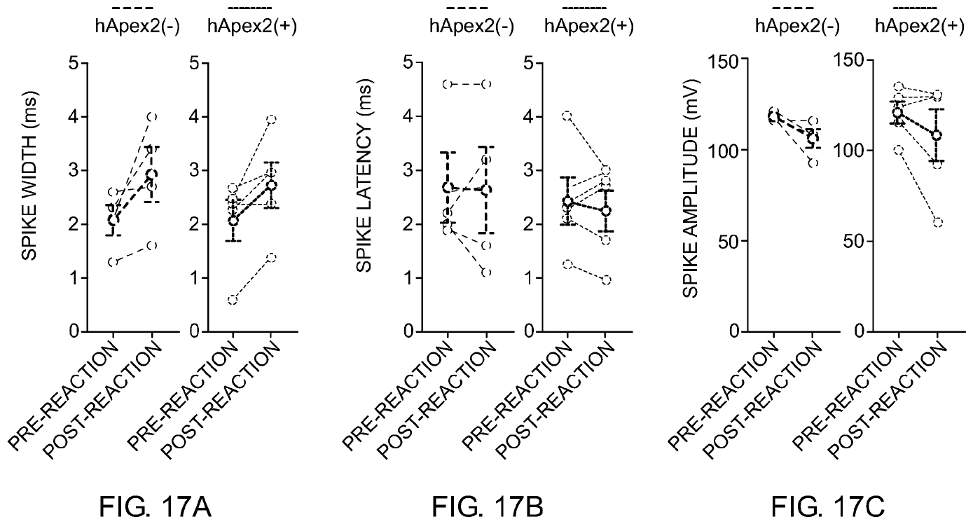

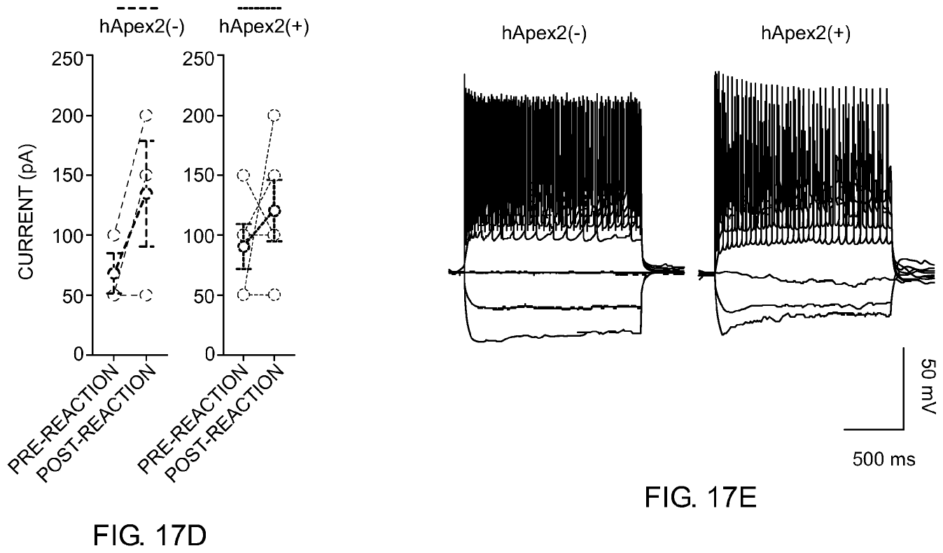

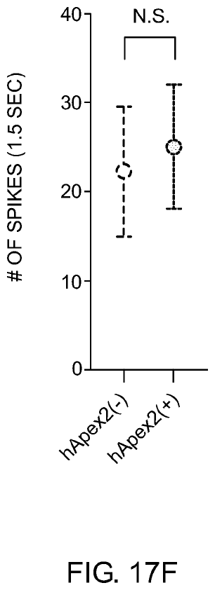

We next studied action potentials evoked with step-waveform current injections (1.5 s duration, with magnitude from −100 pA to 400 pA; FIG. 3I, FIG. 17). hApex2(+) neurons exhibited similar action potential properties before and after polymerization but with decreased current-evoked firing frequency after polymerization (25±6.9 Hz vs 7.8±3.68 Hz), while hApex2(−) neurons showed comparable firing rates pre- and post-polymerization (22.25±7.28 Hz vs 18.33±10.11 Hz) (FIG. 3I-3J). While resting potential and input resistance changes were not observed and thus did not present as possible mechanisms for reduced spike elicitation, experimental and theoretical studies have demonstrated inverse correlation between spike frequency and membrane capacitance (36-38), consistent as well with data here from slice physiology showing strikingly increased capacitance after conductive polymer coating (35.62±6.6 pF vs 52.4±12.65 pF) (FIG. 3F). Taken together, these data suggest that targeted deposition of charged conductive polymers on dielectric lipid bilayers of neurons specifically increases membrane capacitance with expected modulation of spike-firing properties.

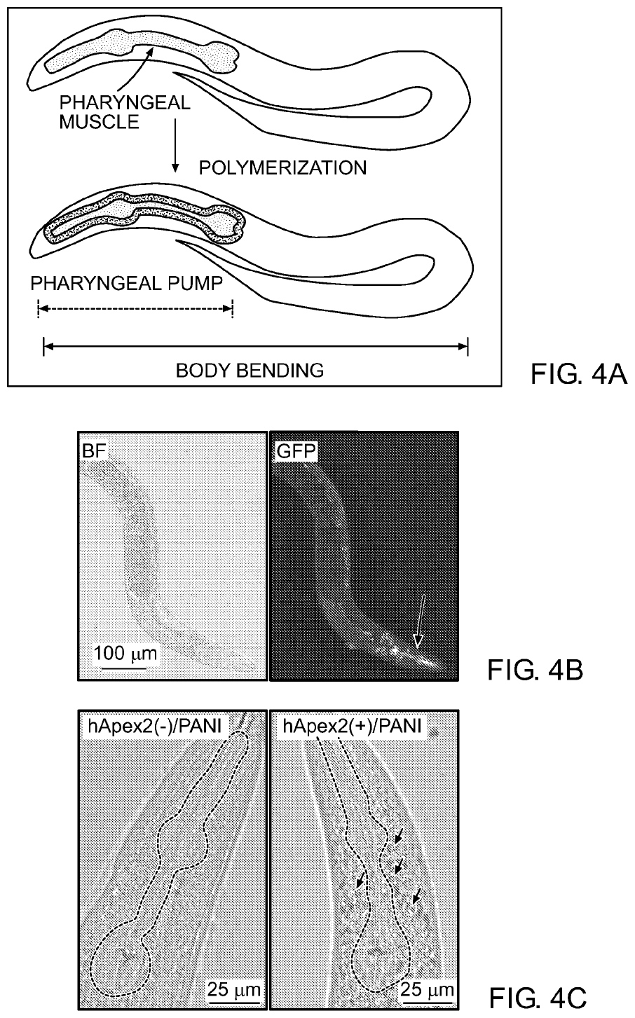

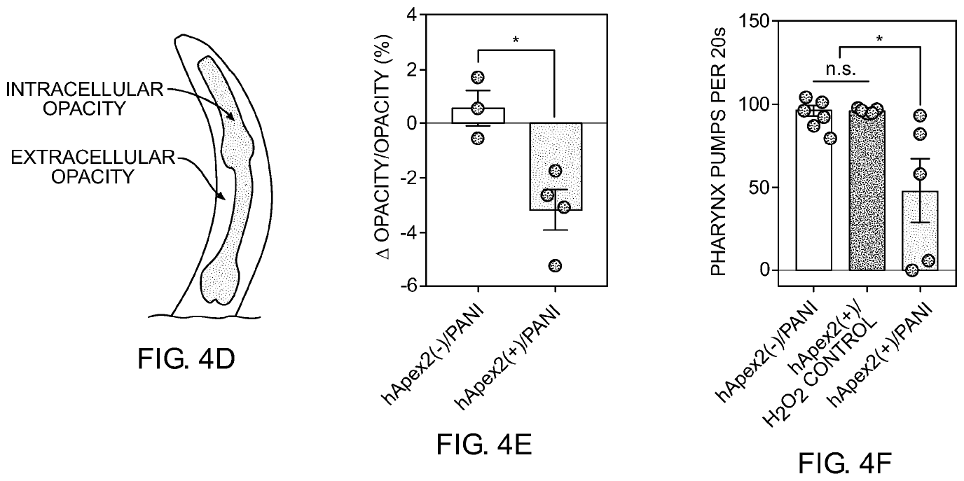

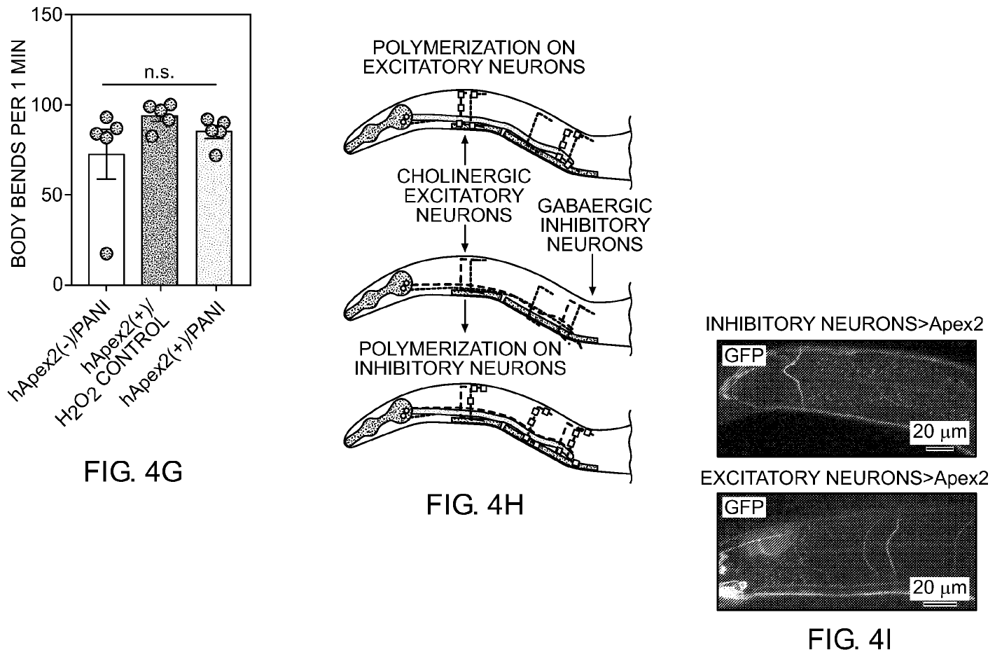

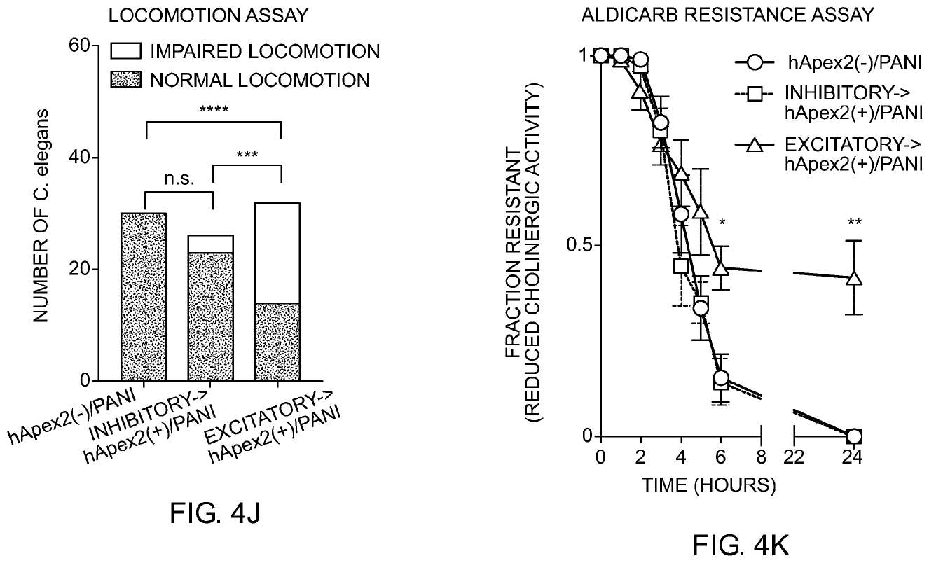

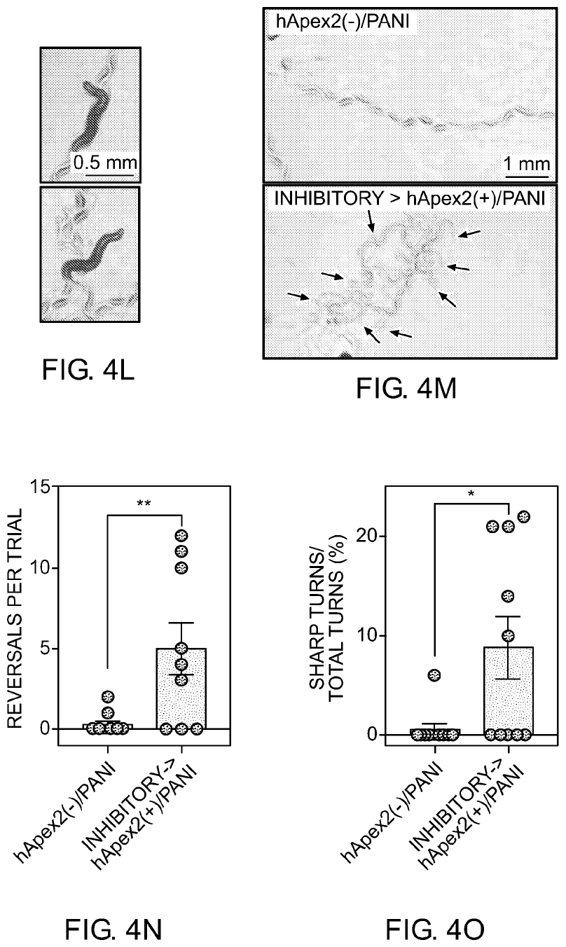

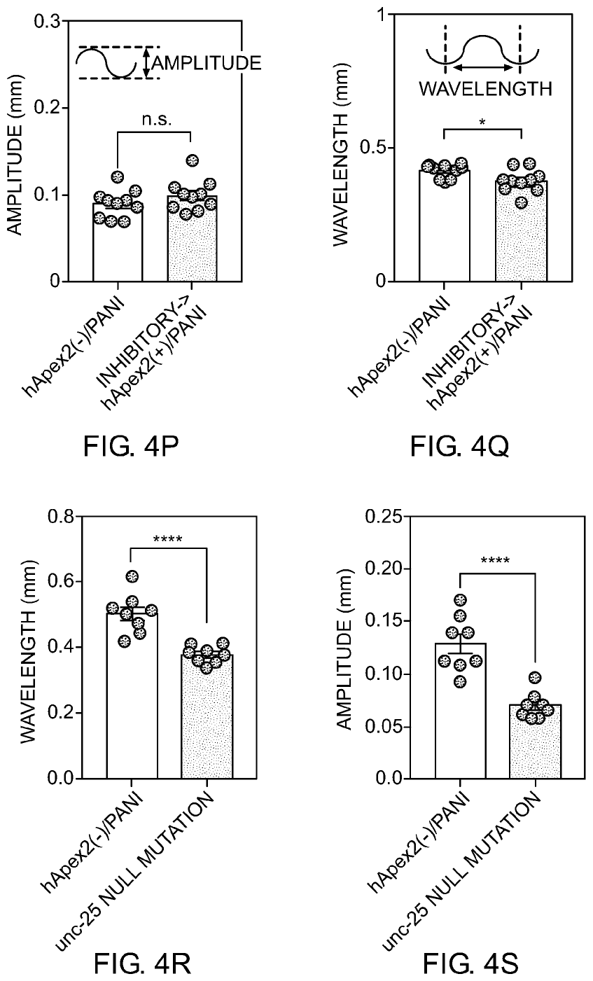

Finally, we tested whether we could specifically modulate behavior in freely moving animals by implementing genetically-targeted conductive polymer formation in vivo. We expressed hApex2-GFP on the membrane of worm (C. elegans) pharyngeal muscle cells (FIGS. 4A-4B). After a 15-minute incubation with polymerization reagents (0.5 mM aniline dimer/0.5 mM aniline/1 mM H₂O₂; worms had been pre-incubated with 0.5 mM aniline dimer/0.5 mM aniline for 1 day), we observed strong localized polymer formation on the pharyngeal muscle cells (FIG. 4C), whereas wild-type worms (hApex2(−)/PANI) exhibited no polymerization (FIGS. 4C-4E). Consistent with expectations arising from the sign of the effect in mammalian in vivo experiments, the conductive polymer-coated C. elegans (hApex2(+)/PANI C. elegans) exhibited reduced pumping frequency of the pharyngeal muscle (FIG. 4F) but no alteration in other body movements, such as bending (FIG. 4G). This indicates that the cell-type-specific polymerization approach elicited specific behavioral changes consistent with inhibition of targeted cells.

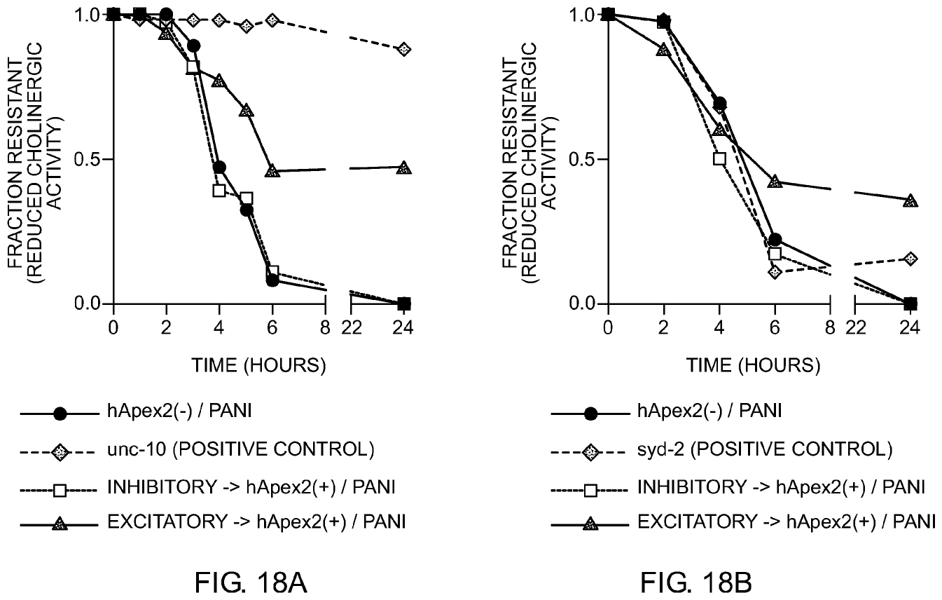

We next expressed hApex2-GFP either in GABAergic inhibitory motor neurons or in cholinergic excitatory motor neurons (FIGS. 4H-4K; Inhibitory→hApex2(+) and Excitatory→hApex2(+), respectively). These two populations were chosen to stringently probe cell-type specificity, since neurites of these two neuron types fasciculate together in the ventral and dorsal nerve cords and synapse both onto each other and the same body wall muscles (FIG. 4H). These two hApex2 strains (FIG. 4I) were polymerized under the conditions described above. After polymerization, Excitatory→hApex2(+)/PANI C. elegans displayed impaired sinusoidal forward locomotion (both spontaneous and aversive-stimulus-evoked), concordant with prior findings after acute optogenetic inhibition of excitatory neurons (39), while sinusoidal forward locomotion of hApex2(−)/PANI and Inhibitory→hApex2(+)/PANI C. elegans was unaffected (FIG. 4J). Also consistent with this pattern. Excitatory→hApex2(+)/PANI C. elegans became more resistant to the acetylcholinesterase inhibitor, aldicarb, while Inhibitory→hApex2(+)/PANI C. elegans and hApex2(−) /PANI C. elegans did not (FIGS. 4K, 18).

While sinusoidal forward locomotion was unchanged for Inhibitory→hApex2(+) worms after polymerization, we examined the behavioral repertoire of these worms in more detail (FIGS. 4L-4Q), since acute optogenetic manipulation of inhibitory neuron activity has been shown to specifically induce sharper turns (40). Indeed, Inhibitory→Apex2(+)/PANI C. elegans exhibited increased reversal frequency (FIG. 4N) and performed sharper (<90°) turns than hApex(−)/PANI worms (FIG. 4O) during movement. Specificity of this effect is underscored by the fact that inhibitory→Apex2(+)/PANI C. elegans maintained the capability to move forward in a straight sinusoidal wave of unchanged amplitude (FIG. 4P) and only minimally reduced wavelength (FIG. 4Q). This conserved sinusoidal movement pattern contrasts with that of worms in which inhibitory neuron function is completely ablated (unc-25 null mutation), which show greatly reduced sinusoidal wave amplitude (FIGS. 4R-4S) (41). Taken together, these behavioral results revealed inhibited, rather than ablated, motor neuron function after polymerization, concordant with electrophysiological characterization in mammalian neurons, and confirming cell-type specificity and biocompatibility in behaving animals.

This approach (selecting and guiding individual cells to carry out chemical synthesis and assembly of new functional structures) we term genetically-targeted chemical assembly (GCA). In this case of metazoan nervous systems, we achieved genetically-targeted in situ chemical synthesis of electroactive polymers via integration of enzyme-facilitated polymerization (in which the catalyst was anchored on the extracellular membrane) with a novel chemical reaction using aniline dimer as the initiator for efficient biocompatible polymerization of polyaniline—together enabling localized in situ synthesis of conductive polymers on genetically-specified cellular elements within living organisms. Absorption/NEXAFS spectra, multimodal imaging, and electrophysiology confirmed chemical, structural, and electronic properties of the conductive polymers per design within diverse biological systems, and cellular viability was verified after polymerization along with successful modulation of membrane properties in living neurons. The cell-type targeting capability of this chemical synthesis was shown to allow cell type-specific modulation of behavior in living animals.

Substantial diversity of variations and applications may be possible in future work. For example, targeted structural deposition, whether inside or outside the cell, could be developed as a means to structurally record or memorialize the form or wiring of cells within a given tissue such as a brain, for later analysis. Distinct strategies for targeting sensitivity to, and triggering of, initiating chemical synthesis could be implemented beyond the radical-based oxidative targeting shown here, including information modalities relating to pH, light, heat, and other chemical or energetic signals. Many other formulations and variants of cellularly-synthesized polymers, and other cell-specific chemical syntheses, may be developed for diverse type and capability of functional characteristics in synthesized structures; for example, different cell types could be instructed to simultaneously create distinct genetically-defined structures in the same preparation, whereupon chemical interfaces could be applied to span one type of cell to another in synapse-like fashion based on chemical moieties available for interface, and that if appropriately functionalized could link to external electronics or to implanted long-shank, high recording contact-density, silicon electrodes (42) as a cell type-specific electrical interface. Bridging across damaged structures with synthesized conductive polymers could become interesting in therapeutics, for example in spinal cord injury repair. Potential challenges will need to be addressed along the way; for example, conductive polymers might over time be internalized by living cells, which could be useful in some contexts but also could result in cytotoxicity. Creation of chemical moiety-defined spaces between cell and structure, or other protective modifications, could modulate internalization, and advanced electronics may further enable next-generation high-performance genetically-targeted bioelectronics.

### REFERENCES

1. A. L. Hodgkin, A. F. Huxley, A quantitative description of membrane current and its application to conduction and excitation in nerve. J. Physiol. 117, 500-544 (1952).

2. K. Deisseroth, Optogenetics: 10 years of microbial opsins in neuroscience. Nat. Neurosci 18, 1213-1225 (2015).

3. C. K. Kim, A. Adhikari, K. Deisseroth, Integration of optogenetics with complementary methodologies in systems neuroscience. Nat. Rev. Neurosci. 8, 222-235 (2017).

4. R. Prakash et al., Two-photon optogenetic toolbox for fast inhibition, excitation, and bistable modulation. Nat. Methods 9, 1171-1179 (2012).

5. J. P. Rickgauer, K. Deisseroth, D. W. Tank, Simultaneous cellular-resolution optical perturbation and imaging of place cell firing fields. Nat. Neurosci. 17, 1816-1824 (2014).

6. L. Carrillo-Reid, W. Yang, Y. Bando, D. S. Peterka, R. Yuste, Imprinting and recalling cortical ensembles. Science 353, 691-694 (2016).

7. J. H. Jennings et al., Interacting neural ensembles in orbitofrontal cortex for social and feeding behavior. Nature 565, 645-649 (2019).

8. T. Dvir et al., Nanowired three-dimensional cardiac patches. Nat. NanotechnoL 6, 720-725 (2011).

9. G. Cellot et al., Carbon nanotubes might improve neuronal performance by favouring electrical shortcuts. Nat. NanotechnoL 4, 126-133 (2009).

10. Y. Jiang, B. Tian, Inorganic semiconductor biointerfaces. Nat. Rev. Mater. 3, 473-490 (2018).

11. V. Gradinaru, J. Treweek, K. Overton, K. Deisseroth, Hydrogel-tissue chemistry: principles and applications. Annu. Rev. Biophys. 47, 355-376 (2018).

12. K. Deisseroth, A look inside the brain. Scientific American 315, 30-37 (2016).

13. K. Chung et al., Structural and molecular interrogation of intact biological systems. Nature 497, 332-337 (2013).

14. B. Yang et al. Single-cell phenotyping within transparent intact tissue through whole-body clearing. Cell 158, 945-958 (2014).

15. J. Niu et al. Engineering live cell surfaces with functional polymers via cytocompatible controlled radical polymerization. Nat. Chem. 9, 537-545 (2017).

16. F. Chen, P. W. Tillberg, E. S. Boyden, Expansion microscopy. Science 347, 543-548 (2015).

17. X. Wang et al., Three-dimensional intact-tissue sequencing of single-cell transcriptional states. Science 361, eaat5691 (2018).

18. L. Ouyang, C. L. Shaw, C. C. Kuo, A. L. Griffin, D. C. Martin, In vivo polymerization of poly(3,4-ethylenedioxythiophene) in the living rat hippocampus does not cause a significant loss of performance in a delayed alternation task. J. Neural. Eng. 11, 026005 (2014).

19. L. Pan et al., Hierarchical nanostructured conducting polymer hydrogel with high electrochemical activity. Proc. Natl. Acad. Sci. USA 109, 9287-9292 (2012).

20. R. Cruz-Silva et al., Template-free enzymatic synthesis of electrically conducting polyaniline using soybean peroxidase. Eur. Poly. J. 41, 1129-1135 (2005).

21. S. Alvarez, S. Manolache, F. Denes, Synthesis of polyaniline using horseradish peroxidase immobilized on plasma-functionalized polyethylene surfaces as initiator. J. Appl. Poly. Sci. 88, 369-379 (2003).

22. S. S. Lam et al., Directed evolution of APEX2 for electron microscopy and proteomics. Nat. Methods 12, 51-54 (2015).

23. T. Scherf et al., A b-hairpin structure in a 13-mer peptide that binds a-bungarotoxin with high affinity and neutralizes its toxicity. Proc. Natl. Acad. Sci. U.S. 98, 6629-6634 (2001).

24. A. S. Pavitt, E. K. Bylaska, P. G. Tratnyek, Oxidation potentials of phenols and anilines: correlation analysis of electrochemical and theoretical values. Environ. Sci. Processes Impacts 19, 339-349 (2017).

25. Y. Wei, Y. Sun, G. W. Jang, X. Tang, Effect of p-Aminodiphenylamine on electrochemical polymerization of aniline. J. Poly. Sc. Part C: Poly. Lett. 28, 81-87 (1990).

26. S. Moulton et al., Polymerisation and characterisation of conducting polyaniline nanoparticle dispersions. Curr. Appl. Phys. 4, 402-406 (2004).

27. C. Henning, K. H. Hallmeier, R. Szargan, XANES investigation of chemical states of nitrogen in polyaniline. Syn. Metals 92, 161-166 (1998).

28. Y. Luo et al. Sub-electron-volt chemical shifts and strong interference effects measured in the resonance x-ray scattering spectra of aniline. Phys. Rev. A 52, 3730-3736 (1995).

29. M. Magnuson et al. The electronic structure of polyaniline and doped phases studied by soft X-ray absorption and emission spectroscopies. J. Chem. Phys. 111, 4756-4761 (1999).

30. D. J. Stokes, Principles and practice of variable pressure/environmental scanning electron microscopy. John Wiley & Sons, UK (2008).

31. E. Stavrinidou et al. Electronic plants. Sci. Adv. 1, e1501136 (2015).

32. A. M. Pasca et al., Functional cortical neurons and astrocytes from human pluripotent stem cells in 3D cultures. Nat. Methods 12, 671-678 (2015).

33. S. A. Sloan, J. Andersen, A. M. Pasca, F. Birey, S. P. Pasca, Generation and assembly of human brain region-specific three-dimensional cultures. Nat. Protoc. 13, 2062-2085 (2018).

34. R. C. Van Lehn et al., Effect of particle diameter and surface composition on the

35 spontaneous fusion of monolayer-protected gold nanoparticles with lipid bilayers. Nano Lett. 13, 4060-4067 (2013).

35. R. P. Carney et al., Electrical method to quantify nanoparticle interaction with lipid bilayers. ACS Nano 7, 932-942 (2013).

36. M. J. Gillespie, R. B. Stein, The relationship between axon diameter, myelin thickness and conduction velocity during atrophy of mammalian peripheral nerves. Brain Res. 259, 41-56 (1983).

37. D. K. Hartline, D. R. Colman, Rapid conduction and the evolution of giant axons and myelinated fibers. Curr. Biol. 17, 29-35 (2007).

38. B. Howell, L. E. Medina, W. M. Grill, Effects of frequency-dependent membrane capacitance on neural excitability. J. Neural. Eng. 12, 056015 (2015).

39. H. E. Kato et al., Structural mechanisms of selectivity and gating in anion channelrhodopsins. Nature 561, 349-354 (2018).

40. J. L. Donnelly et al., Monoaminergic orchestration of motor programs in a complex C. elegans behaviour. Plos Biol. 4, e1001529 (2013).

41. S. L. McIntire, E. Jorgensen, J. Kaplan, H. R. Horvitz, The GABAergic nervous system of Caenorhabditis elegans. Nature 364, 337-341 (1993).

42. J. J. Jun et al., Fully integrated silicon probes for high-density recording of neural activity. Nature 551, 232-236 (2017).

### EXAMPLE 2

### Materials and Methods

### Oligomer Synthesis

Sodium 4-((5,7-di(thiophen-2-yl)-2,3-dihydrothieno[3,4-b][1,4]dioxin-2-yl)methoxy)butane-1-sulfonate (termed TETs in paper) was synthesized using the following steps. Commercial reactants were used without further purification unless stated otherwise. All the solvents used in the reaction were taken out of solvent purification system. (5,7-di(thiophen-2-yl)-2,3-dihydrothieno[3,4-b][1,4]dioxin-2-yl)methanol (1) were synthesized according to the previous reports (1). Sodium 4-((5,7-di(thiophen-2-yl)-2,3-dihydrothieno[3,4-b][1,4]dioxin-2-yl)methoxy)butane-1-sulfonate (2) was synthesized according to the scheme shown in FIG. 11A. This reaction was carried out using similar conditions as a previous report (2). A two necked round-bottom flask equipped with a magnetic stir bar and reflux funnel was charged with 1 (2.6 g mmol, 7.7 mmol) and dry toluene (30 mL) under N₂. Sodium hydride (0.38 g, 60%, 9.4 mmol) was added at room temperature. After stirring 30 minutes, 1,4-butane sultone (1.1 g, 8.4 mmol) was added dropwise by syringe and the solution was stirred for 10 min at room temperature. The solid was corrected by filter and washed by toluene and then acetone under N₂ flow to afford compound 2 as a dark yellow solid (3.2 g, 84% yield). ¹H NMR (400 MHz, CDCl₃, 298 K): 6.94, 6.76, 6.70, 6.66, 6.62, 6.52, 3.82, 3.64, 3.41, 2.53, 1.39, 1.19. ¹³C NMR (100 MHz, CDCl3, 298 K): 137.1, 137.0, 134.2, 134.1, 127.6, 127.6, 124.3, 124.4, 123.1, 122.9, 109.6, 109.2, 73.2, 71.3, 68.7, 65.9, 51.0, 28.0, 21.1.

### Viral Vector Construction

Viral vectors based on adeno-associated virus (AAV) under the control of the human

Synapsin (hSyn) promoter were generated as previously described and in Biosafety Level 2-certified tissue culture facilities in 1-2 weeks.
(1) eYFP-Apex2: Apex2 was cloned from pcDNA3 Connexin43-GFP-Apex2 (addgene plasmid #49385) (3).
(2) hApex2-eYFP: Human codon-optimized Apex2 (hApex2) was fused to eYFP (3).
(3) BTX-hAPex2-eYFP: A a-bungarotoxin motif (BTX) was attached at the 5′end of hApex2-eYFP following previous report (4).
(4) hApex2-eYFP-GPI: A glycosyl phosphatidyl inositol (GPI) signal was attached at the 3′ end of eYFP for hApex2-eYFP-GPI following previous report (5).
(5) BTX-hApex2-p2A-hChR2(H134R)-eYFP: The ribosomal skip site p2A was inserted after hApex2, and before hChR2(H134R)-eYFP to yield BTX-hApex2-p2A-hChR2(H134R)-eYFP (6).

### Polymer Precursor Solutions

To generate polymers utilizing hApex2 in cells as an enzymatic catalyst, a polymer precursor solution was prepared as a combination of a monomer stock solution and hydrogen peroxide stock solution. All solutions were prepared fresh each time.

Aniline-aniline dimer stock solution was prepared by dissolving 0.5 mM aniline (242284 Sigma-Aldrich) and 0.5 mM N-phenyl-1,4-phenylenediamine (aniline dimer) (241393, Sigma-Aldrich) in 1X buffer solution (1× PBS, Tyrode or aCSF solution depending on the neural systems) for at least 2 hours at room temperature using a magnetic stir bar. Specifically, aniline dimer needed to be 1) dissolved as 100 mM stock solution in deionized water, 2) neutralized by 100 mM hydrochloride and 3) further diluted in 1× buffer solutions. The precursor solution was ready for reaction when there were no visible chunks in the solution, and had the appearance of a uniform, light-green liquid. The pH of the solution was adjusted to 7.35 by 10 mM NaOH solution. The hydrogen peroxide stock solution was prepared by diluting 100 mM H₂O₂ 1 × buffer solution. When ready to perform reaction, 1 to 100 μL of H₂O₂ stock solution and 10 mL of aniline-aniline dimer stock solution was combined to form the polymer precursor solution applied to the samples.

TETs-aniline dimer stock solution was prepared by dissolving 0.5 mM aniline dimer and 0.5 mM TETs in 1× buffer solution for 10 min at room temperature using a magnetic stir bar. The same concentration of H₂O₂ stock solution was used to prepare the correct concentration of H₂O₂ in stock solution.

### Cell and Tissue Sample Preparation

### Neuronal Culture, Transfection, and Cell-Intact Staining

Primary culture hippocampal rat neurons were prepared as previously described (6). The hippocampus of Spague-Dawley rat pups (Charles River) was removed at postnatal day 0 (P0), where CA1 and CA3 regions were digested with 0.4 mg/mL papain (Worthington, Lakewood, N.J.) and plated onto 12 mm glass coverslips pre-coated with 1:30 Matrigel (Beckton Dickinson Labware). Cells were plated in 24-well plates, at a density of 65,000 cells per well. The cultured neurons were maintained in Neurobasal-A medium (Invitrogen) containing 1.25% FBS (Fisher Scientific), 4% B-27 supplement (Gibco), 2 mM Glutamax (Gibco) and 2 mg/mL fluorodeoxyuridine (FUDR, Sigma), and kept in a humid culture incubator with 5% CO₂ at 37° C.

Primary culture neurons were transfected 6-10 days in vitro (DIV) with various Apex2 constructs. For each well to be transfected, a DNA-CaCl₂ mix containing with the following reagents was prepared: 2 μg of DNA (prepared using an endotoxin-free preparation kit (Qiagen)) 1.875 μL 2 M CaCl₂, and sterile water added for a total volume of 15 μL. An additional 15 μL of 2× filtered HEPES-buffered saline (HBS, in mM: 50 HEPES, 1.5 Na₂HPO₄,

280 NaCl, pH 7.05 with NaOH) was added, and the resulting 30 μL mix was incubated at room temperature for 20 minutes. In the meanwhile, the neuronal growth medium was taken out of the wells and kept at 37° C., and was replaced with 400 μL pre-warmed minimal essential medium (MEM). The DNA-CaCl₂-HBS mix was then added dropwise into each well, and the plates were transported to the culture incubator for 45-60 minutes. Each well was then washed three times with 1 mL of pre-warmed MEM, after which the MEM was removed and the original neuronal growth medium was added back into the wells. The transfected neuronal culture plates were placed in the culture incubator for another 6 days.

To verify extracellular expression of hApex2 with cell membrane fully intact, cultured neurons expressing different hApex2-eYFP constructs were stained with primary antibody against green fluorescence protein (GFP) prior to paraformaldehyde (PFA) fixation. Cultured neurons were washed 3 times with 1 mL of pre-warmed serum free Neurobasal medium (Invitrogen) supplemented with 4% B-27 (Gibco) and 2 mM Glutamax (Gibco) and incubated at 37° C., 5% CO₂ for one hour in 1 mL serum free media with rabbit anti-GFP (Thermo Fisher) at 1:200 dilution. Following the primary antibody treatment, the neurons were washed 3 times with serum-free media to remove any excess antibody and fixed with 4% PFA at room temperature for 15 minutes. The cells were washed 3 times with PBS, blocked with PBS containing 3% donkey serum and 0.03% Triton X100 (blocking solution) for 1 hour at room temperature and then exposed for 1 hour at room temperature to Alexa 647 Goat anti-rabbit secondary antibody (Abcam) diluted 1:500 in blocking solution. The cover slips were washed three times with 1× PBS containing 0.03% Triton X100 and mounted on slides using (insert here). All images were taken on a Leica confocal at 40× with matched setting.

### HEK Cell Culture and Transfection

Human embryonic kidney cell cultures (HEK-293: ATCC® CRL-1573™) were maintained in 50 mL Dulbecco's Modified Eagle Medium (Life Technologies) containing 100 units/mL of penicillin and 100 μg/mL of streptomycin as well as fetal bovine serum at a dilution of 1:10. HEK cells were grown in incubators at 37° C./5% CO₂ and were transferred to a new 225 cm² culture flask (Thermo) every 3 to 4 days at passaging dilutions ranging from 1:5 to 1:8. 24 h prior to DNA transfections cells were plated on 2 cm poly-D-lysine coated glass cover slips and maintained in 24 well culture plates (Thermo) with 500 μL growth medium. 24 h prior to recordings, HEK cells were transfected with 1.6 μg plasmid DNA per well using 2 μL Lipofectamine 2000 (Life Technologies).

Generation of Human Cortical Spheroids (hCS) and Viral Infection

Generation of hCS from human induced pluripotent stem cells (hiPSC) was performed as previously described (7, 8). Briefly, hiPSCs were exposed to a low concentration of dispase (Invitrogen: 17105-041; 0.7 mg/mL) for ˜30 min. Suspended colonies were subsequently transferred into ultra-low-attachment 100 mm plastic plates (Corning) in hiPSC medium without FGF2. For the first 24 h (day 0), the medium was supplemented with the ROCK inhibitor Y-27632 (EMD Chemicals). For neural induction, dorsomorphin (also known as compound C; Sigma 5 μM) and SB-431542 (Tocris, 10 μM) were added to the medium for the first five days. On the sixth day in suspension, the floating spheroids were moved to neural medium (NM) containing Neurobasal (Invitrogen: 10888), B-27 supplement without vitamin A (Invitrogen: 12587), GlutaMax (Invitrogen, 1:100), 100 U/mL penicillin and 100 μL streptomycin (Invitrogen). The NM was supplemented with 20 ng/ml FGF2 (R&D Systems) and 20 ng/ml EGF (R&D Systems) for 19 days with daily medium change in the first 10 days, and every other day for the subsequent 9 days. To promote differentiation of the neural progenitors into neurons, FGF2 and EGF were replaced with 20 ng/mL BDNF (Peprotech) and 20 ng/mL NT3 (Peprotech) starting at day 25, while from day 43 onwards only NM without growth factors was used for medium changes every four days.

At day 98-132 of in vitro neural differentiation, 2-3 hCS were pooled in a single tube with 250 μL NM and incubated with either AAV8-hSyn-eYFP or AAVdj-hSyn-BTX-hApex2-eYFP overnight. The viral titer per tube ranged from 4×10¹² to 8×10¹². The hCS were then transferred to low attachment plates the next day and media was changed. The viral expression was evident after 7-10 days.

### Acute Brain Slice

### Stereotactic Injection in the Rodent Brain

All surgeries were performed under aseptic conditions as previously described (9) and according to protocols approved by the Stanford Administrative Panel on Laboratory Animal Care and Animal Care and Use Committee of Stanford University.

Adult wildtype female mice aged 10 weeks (Strain C57BL/6J #664, Jackson Laboratory, Maine, USA) were anesthetized with oxygen/isoflurane inhalation and a subcutaneous injection of 0.05-0.1 mg/kg Buprenorphine (Sigma). Fur was sheared from the top of the animal's head and the head was placed in a stereotactic apparatus (David Kopf Instruments) attached to oxygen and isoflurane flow. Lubricant eye ointment was applied (Pharmaderm). A midline scalp incision was made and 0.5 mm diameter craniotomies were drilled for bilateral stereotactic injection into primary motor cortex region (mediolateral (ML): +/−0.86, anteroposterior (AP): +/−0.38 mm and dorsoventral (DV): −1.5 mm) and hippocampal region (ML: +1.25, AP: −1.3, DV: −1.75 and ML: +1.4, AP: −2.4, DV: −1.7) using a high-speed micro drill (Fine Science Tools). AAVdj-hSyn-eYFP, AAVdj-hSyn-BTX-hApex2-eYFP (titer=2.21×10¹³) was delivered to each injection site via a 10 μL Hamilton syringe and a thin 34-gauge metal needle; mounted onto a micro-pump (WPI UltraMicroPump III, WPI). The injection volume and flow rate were 1 μL at 0.1 μL/min. After injection, the needle was left in place for an additional 10 minutes to allow the virus to diffuse into the brain tissue, and withdrawn slowly afterwards. Skin was resealed using Vetbond surgical adhesive. All mice were housed after surgery and recovered for at least 2 weeks before experiment.

### In Situ Polymerization Procedure

Several characterization experiments were performed to validate the synthesis of PANI on the cell membrane via hApex2 expression. For all imaging and chemical characterization experiments, both neurons and hCS were fixed in 4% paraformaldehyde (PFA) prior to polymerization reaction. All steps took place at room temperature.

### Fixed Cultured Neurons Polymerization:

(1) PANI polymerization: Neurons cultured on glass coverslips expressing eYFP (named as hApex2(−) neuron) and BTX-hApex2-eYFP (named as hApex2(+) neuron) were fixed in 4% PFA for 15 min and washed 3 times with 1× PBS. Depending on the experiment, neurons were exposed to polymer precursor solution with 0.01 to 1 mM H202, 0.5 mM aniline dimer and aniline for 0.5 to 3 hours on a shaker (slow speed) and washed 3 times with 1× PBS. Reactivity was assessed using brightfield imaging to detect light-absorbing particles from PANI deposition.

(2) Control experiments for aniline and aniline dimer reactivity: To optimize reactivity (reduce oxidation potential) of polymer precursor solution with cell-expressed hApex2 enzymatic catalyst, fixed neurons were exposed to either aniline solution (1 mM aniline and 0.1 mM H₂O₂ were dissolved in PBS solution) or aniline-dimer solution (1 mM aniline dimer and 0.1 mM H₂O₂ were dissolved in PBS solution) for 30 min and then washed 3× with 1× PBS.

### Living Neuron Polymerization

(1) Control experiment for extracellular hApex2-specific reaction: To verify the specificity of the PANI reaction to hApex2 expressed extracellularly to the cell membrane, fixed hApex2(+) neurons with cell-intact staining of hApex2-eYFP (blocking chemical access to the enzyme) were exposed to polymer precursor solution with 0.1 mM H2O2 for 0.5 hours, and then washed 3 times with 1× PBS.

(2) Living neurons for condition testing: hApex2(+) and hApex2(−) neurons cultured on glass coverslips were exposed to Tyrode polymer precursor solution (125 mM NaCl, 2 mM KCl, 2 mM MgCl₂, 2 mM CaCl₂, 30 mM Glucose, 25 mM HEPES; titrated to pH 7.35 with NaOH and adjusted osmolarity to 298) with (0.05 and 0.01 mM H₂O₂, 0.5 mM aniline dimer and 0.5 mM aniline) for 10 to 30 min on a shaker (slow speed) and washed 3 three times in Tyrode solution before characterization.

### Fixed hCS

hApex2(+) and hApex2(−) hCS were fixed in 4% PFA for 1 hour and washed 3 times in 1× PBS. Fixed hCS were exposed to polymer precursor solution for 0.5 hours, and then washed 3 times in 1× PBS.

Fixed brain slice

Unfixed, freshly prepared brain slices were exposed to polymer precursor solution in aCSF (3 mM KCl, 11 mM glucose, 123 mM NaCl, 1.25 mM NaH₂PO₄, 1 mM MgCl₂ and 2 mM CaCl₂, and adjusted osmolarity to 300) with 0.05 mM H₂O₂ for 0.5 hours and then washed 3 times in aCSF. The post-reacted brain slices were then fixed in 4% PFA overnight at 4° C., and then washed 3 times in 1× PBS.

### Doping the Synthesized Polymers

To enable more accurate chemical analysis of PANI reaction product, an acidic solution was prepared and applied to the post-reacted samples, “doping” the PANI with additional charge carriers. The doping solution was prepared by dissolving p-toluenesulfonic acid (402885, Sigma-Aldrich) in DI water at 100 mM concentration. Post-reacted samples were exposed to the doping solution at room temperature for 30 mins until the color (visually observed) of the cover slips or hCS was changed from purple to green. This indicated that the PANI has been transferred into doped PANI (dPANI). Samples were washed 3 times in 1× PBS to remove extra p-toluenesulfonic acid.

### Morphology and Chemical Structure Characterizations.

### Bright-Field and Epi-Fluorescence Imaging

The post-reacted neurons cultured on the glass coverslips were imaged by Zeiss Axio Imager 2 (Carl Zeiss Microscopy) with 20× water and 63× oil immersion lens.

### Confocal Imaging

All the post-reacted culture neurons, hCS and brain slice samples were imaged by Leica TCS SP8 confocal laser scanning microscope. ProLong Gold Antifade Reagent (Fisher Scientific) was used to embed sample for imaging.

### Ultraviolet-Visible (UV-vis) Spectrophotometry

(1) Culture neurons: All neuron samples on glass coverslips were washed 3 times in 1× PBS and 3 times in DI water, then air-dried and mounted onto the sample holder. Agilent Cary 6000i was used for UV-vis spectrophotometry characterization.

(2) Standard PANI: Commercially available PANI powders (556386, Mw˜50K Sigma-Aldrich) were dispersed into chloroform or DI water at 100 mg/mL and then spin-coated onto glass slides. Samples were air-dried and mounted onto the sample holder. Agilent Cary 6000i was used for UV-vis spectrophotometry characterization.

### X-Ray Photoelectron Spectroscopy (XPS)

All neuron samples were washed 3 times in 1× PBS and 3 times in DI water. Samples were air-dried and mounted onto the sample holder. Elemental composition was measured with XPS (PHI 5000 Versaprobe, Al Kα source).

### Near Edge X-Ray Absorption Fine Structure (NEXAFS) Spectroscopy

All neuron samples were washed 3 times in 1× PBS and 3 times in DI water. Samples were air-dried and mounted onto the sample holder. NEXAFS spectra were collected at the Beamline 11.0.1.2, Advanced Light Source, Lawrence Berkeley National Laboratory (LBNL) (10). Electronic state of Carbon and Nitrogen in the sample was probed by NEXAFS spectroscopy using the bulk sensitive Total Florescence Yield mode. In-Vacuum CCD camera (PI-MTE, Princeton Instrument) was used as florescence detector. The energy resolution of the beamline at the nitrogen k-edge is ˜0.1 eV (E/ΔE>3000). The beam spot is about 150 μm×150 μm. The energy was calibrated using (highly oriented pyrolytic graphite) HOPG absorption and N₂ gas absorption.

### Variable Pressure-Scanning Electron Microscopy (VP-SEM)

Fixed and post-reacted neurons were post-fixed in 4% PFA, rinsed 3× in DI water, and mounted in a thin film of water onto a 10 mm flat stub fitting the in situ Peltier coolstage (Deben, Suffolk, UK) in the Hitachi S-3400N (Hitachi HTA, Dallas, Tex.). Initial stage temperature was set to 4° C., and pressure and temperature correlatively decreased to 60 Pa/−25° C. to limit water-loss while optimizing resolution (11). Visualization was done at 15 kV and 60 Pa using Backscattered Electron (BSE) detection using scan cycle times of 40 seconds.

### Photo-Electron Spectroscopy in Air (PESA)

All neuron samples were washed 3 times in 1× PBS and DI water. Samples were air-dried to remove extra ions and mounted onto the sample holder. Riken AC-2 Photoelectron Spectrometer was used for PESA characterization.

### Electrical Measurement

Fixed and post-reacted hApex2(−)/PANI and hApex2(+)/PANI neurons and HEK cells, and hApex2(−)/PANI-PTETs and hApex2(+)/PANI-PTETs on the glass coverslips were washed 3 times in DI water and air dried to remove extra ions. To define contact electrodes on neuron surface, thermal evaporation was used to deposit a 100 nm-thick Au film on the dried coverslips through shadow (200 μm interelectrode distance). Substrates were kept rotating during evaporation to make a conformal coating of Au electrodes on the surface of fixed neurons. After the initial measurement, both hApex2(−)/PANI and hApex2(+)/PANI samples were exposed to HCl vapor overnight to dope the synthesized PANI into dPANI. The conductivity of all samples was measured using Keithley 4200.

### Viability and Electrophysiology Characterization

### Cell Viability Assay

To optimize biocompatible conditions of hydrogen peroxide reagent, cultured neurons were exposed to polymer precursor in Tyrode's solution as described earlier with varying H₂O₂ concentrations of 0.01 to 1 mM at room temperature. Reaction was terminated after 10 minutes followed by 3 times rinse in Tyrode's solution. After reaction, neurons were stained with 500 μL of a 1:1000 dilution of Propidium Iodide (Thermo Fisher) 1 mg/mL stock in Tyrode's solution for five minutes, followed by 3 times rinse in Tyrode's solution. The cells were then imaged by inverted confocal microscope TCS SP8 confocal laser scanning microscope. ImageJ was used to count the number of dead cells indicated by the red fluorescent nuclei.

### In Vitro Patch Clamp Characterization on Cultured Neurons

Whole-cell patch-clamp recordings of hApex2(−) and hApex2(+) cultured neurons were performed as previously described (12). For the whole-cell recording in cultured neurons, intracellular solution was prepared following the recipe: 140 mM Potassium gluconate, 10 mM HEPES-KOH pH 7.3-7.4, 10 mM EGTA, 2 mM MgCl₂. A different Tyrode solution (150 mM NaCl, 4 mM KCl, 2 mM MgCl₂, 2 mM CaCl₂, 20 mM glucose, 10 mM HEPES; titrated to pH 7.35 with NaOH and adjusted osmolarity to 320-330) was used as extracellular solution. Signals were amplified and digitized using the Multiclamp 700B and DigiData1400 (Molecular Devices), and Leica DM LFSA microscope was used to visualize the cells. Patch pipettes (4-6 MΩ) were pulled using a P2000 micropipette puller (Sutter Instruments). Hippocampal Neurons cultured on glass coverslips were removed from cell media and washed with pre-warmed (room temperature, 22-25° C.) Tyrode's solution. Samples were then exposed to polymer precursor in Tyrode's solution (0.5 mM aniline, 0.5 mM aniline dimer and 0.05 mM H₂O₂) for 10-20 mins at room temperature, then washed with normal Tyrode's solution three times to stop the reaction.

Recordings were performed in the presence of bath-applied glutamatergic synaptic blockers: 6-cyano-7-nitroquinoxaline-2,3,-dione (CNQX; 10 μM, Tocris) for AMPA receptors and D(−)-2-amino-5-phosphonovaleric acid (APV; 25 μM, Tocris) for NMDA receptors. Membrane resistance and cellular capacitance were calculated from 10 mV depolarization under the voltage clamp mode. Then, the mode was switched to current clamp, and action potentials were generated through current injection (500 pA, 10 ms, 5 Hz) while holding at −65 to −70 mV membrane potential.

Analyses of physiological results were performed using ClampFit software (Axon Instruments). Spike width was estimated at the half-peak position from the threshold potential (typically −40 mV) to the peak of a single action potential. Spike latency was estimated as the time point of the peak of an action potential starting from the onset of the current injection. Spike amplitude was estimated as the magnitude of the action potential, taking the resting potential as the baseline.

### In Vivo Patch Clamp Characterization on Brain Slice

Acute slice recordings were performed 2-4 weeks after virus injection. Coronal slices 300 μm in thickness were prepared after intracardial perfusion with ice-cold cutting solution: 93 mM N-methyl-d-glucamine (NMDG), 2.5 mM KCl, 25 mM glucose, 1.2 mM NaH₂PO₄, 10 mM MgSO₄, 0.5 mM CaCl₂, 30 mM NaHCO₃, 5 mM sodium ascorbate, 3 mM sodium pyruvate, 2 mM thiourea and 20 mM HEPES with pH adjusted at 7.3-7.4. Slices were incubated for 12-14 min at 32-34° C. NMDG cutting solution, and then transported to oxygenated artificial cerebrospinal fluid (aCSF) solution at room temperature: 124 mM NaCl, 2.5 mM KCl, 24 mM NaHCO₃, 2 mM CaCl₂, 2 mM MgSO₄, 1.2 mM NaH₂PO₄, 12.5 mM glucose and 5 mM HEPES at pH 7.3-7.4. The aCSF solution also contained synaptic transmission blockers (25 μM APV and 10 μM CNQX) for recordings. Recording patch pipettes contained the following intracellular solution: 140 mM K-gluconate, 10 mM HEPES pH 7.2, 10 mM EGTA and 2 mM MgCl₂.

Slice recording was conducted under the same electrophysiology rig set-up as the in vitro recording experiment. Similarly to the in vitro recording experiments, input resistance and cell capacitance were calculated after a −10-mV depolarization. Then, the mode was switched to current clamp to determine the resting potential of each cell and the holding current to keep the cell's membrane potential at −70 mV, followed by stepwise current injection (from −100 pA to 400 pA) to determine the threshold current for spike generation. Action potentials were induced through pulsed current injection (100-500 pA, 5 ms, 5 Hz) After finishing the recording from hApex2(−) and hApex2(+) neuron, aCSF solution including 1 mM Aniline, 1 mM Dimer and 100 μM H₂O₂ was introduced into the bath together with normal aCSF perfusion for 10 minutes to enable the in situ polymerization, while maintaining the whole-cell recording. Then, the entire electrophysiological characterization was repeated for the same neuron after polymerization.

C. elegans Strain Construction and Behavior

C. elegans strains were cultured on E. coli OP50 as described (13). The wild-type N2 strain was microinjected with pCER254(Pmyo-2:signalpeptide:hApex2:mcd8:GFP) at 1 ng/μL, pCER256(Punc-47:signalpeptide:hApex2:mcd8:GFP) at 5 ng/μL, or pCER264(Punc-17:signalpeptide:hApex2:mcd8:GFP) with Podr-1:RFP as a co-injection marker (75 ng/μL) to make transgenic hApex(+) strains. hApex2(−) vs. hApex(+) worms were transferred to plates containing (1 mL of stock solution containing 0.5 mM aniline dimer and 0.5 mM aniline with final volume at 10 mL) as L4s, and all experiments were performed the next day. Pre-incubated worms were soaked in the polymer precursor containing (0.5 mM aniline dimer and 0.5 mM aniline), then returned to standard OP50 plates prior to being imaged for polymer deposition or assayed for behavior. Behavior assays were performed on OP50 plates, except for body bending (FIG. 5H), which was performed in M9 solution. To quantify movement parameters in FIG. 5K and FIG. 5M-R, worms were placed individually on plates to produce tracks. Worms that did not initially produce a straight track (e.g. FIG. 5M lower) were transferred to a fresh patch of OP50 until a straight track of at least 9 consecutive bends was produced, and these straight tracks were used to quantify wavelength (FIG. 5L) and amplitude (FIG. 5M). Worms that did not produce a track of two consecutive straight body bends, even upon prodding with a platinum wire, were scored as “cannot move” in FIG. 5K. Images of tracks were acquired using a Zeiss Axioplan 2 microscope with a 2.5× objective and analyzed using ImageJ software. Aldicarb assays were performed as described (14). Briefly, 25 worms of each were placed on plates containing 0.7 mM aldicarb and assayed for acute paralysis, defined as lacking movement of the body after being prodded three times on the head and tail. hApex2(−) and hApex(+) strains were assayed in four experiments, and the positive control mutants for aldicarb resistance syd-2 (weakly resistant) and unc-10 (strongly resistant) each used in two of those experiments. All behavior assays were scored blind to genotype.

### Data Analysis

pClamp 10.6 (Molecular Devices), and Prism 7 (GraphPad) software were used to record and analyze data. Statistical analyses were performed with two-tailed unpaired t-test or one-way ANOVA.

All molecular graphics figures were prepared with Cuemol (cuemol.org). For channelrhodopsin, crystal structure of a cation channelrhodopsin C1C2 (PDB ID: 3ug9, 15) was used, for hApex2, ascorbate peroxidase structure (PDB ID: 1 oag, 16) was used and for the membrane tags, 7th transmembrane domain alpha helix of C1C2 structure was used.

### REFERENCES

1. C. Liu, et al. Palladium-catalyzed direct C-H arylations of dioxythiophenes bearing reactive functional groups: a step-economical approach for functional 7-conjugated oligoarenes. Org. Biomol. Chem. 13, 8505-8511 (2015).

2. S. Haraguchi et al. Control of polythiophene redox potentials based on supramolecular complexation with helical schizophyllan. Chem. Commun. 40, 6086-6088 (2009).

3. S. S. Lam et al. Directed evolution of APEX2 for electron microscopy and proximity labeling. Nature Methods 12, 51-54 (2015).

4. C. M. McCann et al. Peptide tags for labeling membrane proteins in live cells with multiple fluorophores. BioTechniques 38, 945-952 (2005).

5. J. Dolan et al. The extracellular Leucine-Rich Repeat superfamily; a comparative survey and analysis of evolutionary relationships and expression patterns. BMC Genomics 10, 230 (2009).

6. V. Gradinaru et al. Molecular and cellular approaches for diversifying and extending optogenetics. Cell 141, 1-12 (2010).

7. A. M. Pasca et al. Functional cortical neurons and astrocytes from human pluripotent stem cells in 3D culture. Nat Methods 12, 671-678 (2015).

8. S. A. Sloan, J. Andersen, A. M. Pasca, F. Birey, S. P. Pasca, Generation and assembly of human brain region-specific three-dimensional cultures. Nat. Protoc. 13, 2062-2085 (2018).

9. F. Zhang et al. Optogenetic interrogation of neural circuits: technology for probing mammalian brain structures. Nat. Protocol. 5, 439 (2010).

10. E. Gann et al. Soft x-ray scattering facility at the Advanced Light Source with real-time data processing and analysis. Rev. Sci. Instrum. 83, 045110 (2012).

11. L. M. Joubert, Visualization of hydrogels with Variable-Pressure SEM. Microsc. MicroanaL 15, 1308-1309 (2009).

12. Y. Kim et al. Crystal structure of the natural anion-conducting channelrhodopsin GtACR1. Nature 561, 343-348 (2018).

13. S. Brenner, The genetics of Caenorhabditis elegans. Genetics 77, 71-94 (1974).

14. T. R. Mahoney et al. Analysis of synaptic transmission in Caenorhabditis elegans using an aldicarb-sensitivity assay. Nature Protocols 1, 1772-1777 (2006).

15. H. E. Kato et al. Crystal structure of the channelrhodopsin light-gated cation channel. Nature 482, 369-374 (2012).

16. K. H. Sharp, M. Mewies, P. C. E. Moody, E. L. Raven, Crystal structure of the ascorbate peroxidase-ascorbate complex. Nat. Struct. Mol. Biol. 10, 303-307 (2003).

1. A method for in situ polymer synthesis on a membrane or subcellular surface of a cell, the method comprising:
a) expressing a polymerization enzyme on the membrane or subcellular surface; and
b) contacting the cell with one or more polymer precursors and a polymerization initiator, wherein the enzyme catalyzes polymerization of the polymer precursors resulting in production of the polymer on the membrane or the subcellular surface.

2. The method of claim 1, wherein the membrane is a plasma membrane or an organelle membrane.

3. The method of claim 2, wherein the polymerization enzyme is localized to an extracellular side of the plasma membrane.

4. The method of claim 1, wherein the polymer is an electroactive, conductive, or insulating polymer.

5. The method of claim 4, wherein the conductive polymer is a polyaniline (PANI) polymer, a poly(3,4-ethylenedioxythiophene) (PEDOT) polymer, a 4-((5,7-di(thiophen-2-yl)-2,3-dihydrothieno[3,4-b][1,4]dioxin-2-yl)methoxy)butane-1-sulfonate (TETS) polymer, or a polyaniline-polyTETS (PANI-PTETS) polymer.

6. The method of claim 5, wherein the conductive polymer is PANI and the polymer precursors are an aniline monomer and an aniline dimer (N-phenyl-1,4-phenylenediamine).

7. (canceled)

8. The method of claim 1, wherein the cell is a neuron, cardiomyocyte, muscle fiber, or endocrine cell.

9. (canceled)

10. The method of claim 1, wherein the polymerization enzyme is a peroxidase and the polymerization initiator is a peroxide.

11-14. (canceled)

15. The method of claim 1, wherein the polymerization enzyme is provided by a vector comprising a promoter operably linked to a polynucleotide encoding a fusion protein comprising the polymerization enzyme connected to a targeting peptide.

16-18. (canceled)

19. The method of claim 15, wherein the fusion protein further comprises a detectable label.

20. The method of claim 19, wherein the detectable label is a fluorescent or bioluminescent protein.

21. (canceled)

22. The method of claim 15, wherein the fusion protein further comprises a channelrhodopsin.

23. (canceled)

24. The method of claim 1, wherein the cell is a live cell or a fixed cell.

25. The method of claim 1, wherein the cell is in a tissue, an organoid, or a subject.

26-29. (canceled)

30. The method of claim 1, further comprising doping the polymer with an acid.

31. The method of claim 30, wherein the acid is p-toluenesulfonic acid.

32. The method of claim 31, wherein the polymer is an emeraldine salt of polyaniline (PANI).

33. The method of claim 2, wherein the polymer changes the capacitance, conductance, or insulation properties of the plasma membrane or organelle membrane.

34. The method of claim 1, further comprising performing optogenetics, electrophysiological measurements, imaging, or spectroscopy, or a combination thereof on the cell.

35-36. (canceled)

37. The method of claim 4, further comprising attaching an electrode to the conductive polymer, wherein the conductive polymer forms a conductive interface between the cell and the electrode.

38-39. (canceled)

40. The method of claim 37, wherein the electrode is produced by depositing a biocompatible material on the surface of the conductive polymer, wherein the biocompatible material comprises platinum, titanium, silver, gold, graphite or other conductive carbon material, indium tin oxide (ITO), fluorine-doped tin oxide (FTO), or a metal alloy or an oxide comprising at least one of tin, platinum, titanium, silver, or gold.

41-48. (canceled)

49. A method for in situ polymer synthesis in brain tissue, the method comprising:
a) expressing a polymerization enzyme on the plasma membranes of a plurality of neurons in the brain; and
b) contacting the brain tissue with one or more polymer precursors and a polymerization initiator, wherein the enzyme catalyzes polymerization of the polymer precursors resulting in production of the polymer on the plasma membranes of a plurality of neurons in the brain.

50-56. (canceled)

57. A method for in situ polymer synthesis on a plasma membrane of a cell, the method comprising:
a) expressing a peroxidase on an extracellular-facing surface of the plasma membrane; and
b) contacting the cell with one or more polymer precursors and a peroxide, wherein the peroxidase catalyzes production of the polymer on the extracellular-facing surface of the plasma membrane.
